(19) United States
(12) Patent Application Publication
(10) Pub. No.: US 2026/0347931 A1
(43) Pub. Date: Nov. 5, 2026

(54) TWO-COLOR SEQUENCING METHOD AND KIT

(71) Applicants: GENEMIND BIOSCIENCES COMPANY LIMITED, Shenzhen (CN); SHENZHEN JUMAI BIOTECHNOLOGY CO., LTD., Shenzhen (CN)

(72) Inventors: Meirong CHEN, Shenzhen (CN); Bin LIU, Shenzhen (CN); Huan JIN, Shenzhen (CN); Fang CHEN, Shenzhen (CN); Lei SUN, Shenzhen (CN)

(73) Assignees: GENEMIND BIOSCIENCES COMPANY LIMITED, Shenzhen (CN); SHENZHEN JUMAI BIOTECHNOLOGY CO., LTD., Shenzhen (CN)

(21) Appl. No.: 19/648,166

(22) Filed: Apr. 15, 2026

(30) Foreign Application Priority Data
Apr. 16, 2025 (CN) 202510481107.0

(51) Int. Cl. C12Q 1/6874; C12Q 1/6823; C12Q 1/6825; G01N 21/64

(52) U.S. Cl. CPC C12Q 1/6874; C12Q 1/6823; C12Q 1/6825; G01N 21/6428; G01N 21/6456; C12Q 2600/16; G01N 2021/6439

(57) ABSTRACT

The present invention relates to the technical field of biological detection, and in particular to a two-color sequencing method and a kit. In the present invention, through reasonably designing the reversible terminators participating in the reactions in the two-color sequencing method and optimizing the reagents and reaction conditions for the two-color sequencing method, a two-color sequencing method suitable for a single-molecule sequencing platform is obtained. Using the two-color sequencing method in single-molecule sequencing can help to reduce the error ratio, improve the sequencing quality, and shorten the sequencing time.

### CROSS-REFERENCE TO RELATED APPLICATIONS

This application claims the priority benefit of Chinese application serial No. 202510481107.0 filed on Apr. 16, 2025, the entirety of which is hereby incorporated by reference herein and made a part of the specification.

### TECHNICAL FIELD

The present disclosure relates to the technical field of biological detection, and in particular to a two-color sequencing method and a two-color sequencing kit.

### BACKGROUND OF THE INVENTION

Single-molecule sequencing, originally developed by Helicos Biosciences Corporation, achieves the effect of sequencing by synthesis by using a TIRF microscope. Single-molecule sequencing can be categorized into single-color sequencing and multi-color sequencing according to the number of types of nucleotide or analog thereof added in each polymerization reaction in the process of single-molecule sequencing. Multi-color sequencing can further be categorized into two-color sequencing and four-color sequencing.

A single-color sequencing kit only allows addition of one fluorescently labeled mononucleotide each time, and four cycles of reaction are required to complete the reactions of four nucleotides. The single-color sequencing kit has a long sequencing time and a short read. A two-color sequencing kit involves performing sequencing using a combination of dA-Atto647N and dT-Atto532 and a combination of dG-Atto647N and dC-Atto532, and two cycles are required to complete the reactions of four nucleotides. Compared with the single-color sequencing kit, the two-color sequencing kit allows shortening the overall sequencing time by approximately half. However, the sequencing read length and the sequencing time still represent limitations.

### SUMMARY

In view of foregoing, the present disclosure provides a two-color sequencing method and a two-color kit that are based on reversible terminator nucleotides and derivatives thereof.

In order to achieve the above inventive object, the present disclosure provides technical solutions as follows.

A two-color sequencing method is provided, comprising the following steps:
(a) allowing four types of reversible terminator to undergo polymerization reactions with nucleic acid molecules attached to a surface, the four types of reversible terminator each independently comprising: a nucleotide or a derivative thereof, a labeling moiety, and a first cleavable group linking the nucleotide or a derivative thereof and the labeling moiety, wherein the labeling moiety is selected from a fluorescent group and/or a specific binding label, at least two of the four types of modified nucleotide carry a first fluorescent group and a second fluorescent group, which are optically distinguishable, respectively, one or two of the four types of modified nucleotide carry the specific binding label, and the specific binding label is capable of specifically binding to a corresponding ligand;
(b) exciting the fluorescent group to luminesce, and collecting a luminescence signal to obtain a first image;
(c) adding a labeling substance, the labeling substance comprising the ligand and a fluorescent group bound to the ligand, and allowing the ligand of the labeling substance to bind to the specific binding label, wherein the fluorescent group bound to the ligand is selected from the first fluorescent group or the second fluorescent group;
(d) exciting the fluorescent group to luminesce, and collecting a luminescence signal from the fluorescent group to obtain a second image; and
(e) determining the types of reversible terminator participating in the polymerization reactions on the basis of the luminescence signals and a change thereof at the same position in the first image and the second image.

As a possible implementation of the two-color sequencing method of the present application, the labeling moieties of two of the four types of modified nucleotide are the first fluorescent group and the second fluorescent group, which are optically distinguishable, respectively, and the labeling moieties of the other two of the four types of modified nucleotide are specific binding labels with different modes of specific binding, and
in step (c), the labeling substance comprises two types, and the two types of labeling substance carry the first fluorescent group and the second fluorescent group, respectively; or,
two of the four types of modified nucleotide each independently carry the first fluorescent group, one of the four types of modified nucleotide carries the second fluorescent group, and the other of the four types of modified nucleotide carries the specific binding label, and
in step (c), the labeling substance comprises one type, and the labeling substance carries the second fluorescent group.

As a possible implementation of the two-color sequencing method of the present application, three of the four types of modified nucleotide carry the first fluorescent group and the second fluorescent group, which are optically distinguishable, respectively, and the other of the four types of modified nucleotide carries the specific binding label, and one of the two types of modified nucleotide carrying the same fluorescent group comprises a second cleavable group, the second cleavable group being so located as to link the first cleavable group and the labeling moiety, and the second cleavable group having a higher cleavability than the first cleavable group, and
step (c) further comprises: performing a cleavage reaction on the second cleavable group.

In an embodiment, the determining the types of reversible terminator participating in the polymerization reactions on the basis of the luminescence signals and a change thereof at the same position in the first image and the second image comprises:
with respect to a first position where the optical signals remain unchanged in both the first image and the second image and are both indicative of the optical signal of the first fluorescent group, determining the reversible terminator participating in the polymerization reaction at that position to be a first reversible terminator;
with respect to a second position where the optical signals remain unchanged in both the first image and the second image and are both indicative of the optical signal of the second fluorescent group, determining the reversible terminator participating in the polymerization reaction at that position to be a second reversible terminator; and
with respect to a third position and a fourth position, where the optical signals become changed but follow different patterns of change between the first image and the second image, determining the reversible terminators participating in the polymerization reactions at that two positions to be a third reversible terminator and a fourth reversible terminator, respectively.

As a possible implementation of the two-color sequencing method of the present application, the structures of the four types of reversible terminator satisfy one or more of the following combinations 1-4: The four reversible terminators have structures satisfying one or more of the following combinations (1)-(4):
(1) the structure of the first reversible terminator is: first nucleotide or derivative thereof-first cleavable group-first fluorescent group,
the structure of the second reversible terminator is: second nucleotide or derivative thereof-first cleavable group-specific binding label,
the structure of the third reversible terminator is: third nucleotide or derivative thereof-first cleavable group-specific binding label, and
the structure of the fourth reversible terminator is: fourth nucleotide or derivative thereof-first cleavable group-second fluorescent group;
(2) the structure of the first reversible terminator is: first nucleotide or derivative thereof-first cleavable group-first fluorescent group,
the structure of the second reversible terminator is: second nucleotide or derivative thereof-first cleavable group-specific binding label,
the structure of the third reversible terminator is: third nucleotide or derivative thereof-first cleavable group-specific binding label, and
the structure of the fourth reversible terminator is: fourth nucleotide or derivative thereof-first cleavable group-second fluorescent group-specific binding label;
(3) the structure of the first reversible terminator is: first nucleotide or derivative thereof-first cleavable group-first fluorescent group,
the structure of the second reversible terminator is: second nucleotide or derivative thereof-first cleavable group-specific binding label,
the structure of the third reversible terminator is: third nucleotide or derivative thereof-first cleavable group-second fluorescent group, and
the structure of the fourth reversible terminator is: fourth nucleotide or derivative thereof-first cleavable group-second cleavable group-first fluorescent group-specific binding label;
(4) the structure of the first reversible terminator is: first nucleotide or derivative thereof-first cleavable group-first fluorescent group,
the structure of the second reversible terminator is: second nucleotide or derivative thereof-first cleavable group-second fluorescent group-specific binding label,
the structure of the third reversible terminator is: third nucleotide or derivative thereof-first cleavable group-second fluorescent group, and
the structure of the fourth reversible terminator is: fourth nucleotide or derivative thereof-first cleavable group-second cleavable group-first fluorescent group;
wherein in each combination, the first nucleotide or derivative thereof, the second nucleotide or derivative thereof, the third nucleotide or derivative thereof, and the fourth nucleotide or derivative thereof are selected from one of adenine or derivative thereof, thymine or derivative thereof, cytosine or derivative thereof, or guanine or derivative thereof, and the first nucleotide or derivative thereof, the second nucleotide or derivative thereof, the third nucleotide or derivative thereof, and the fourth nucleotide or derivative thereof are different from each other.

On the basis of any one of the above embodiments, as a possible implementation of the two-color sequencing method of the present application, the first cleavable group is selected from groups comprising at least one of —N3-, —CH₂F, —C(O)NHBu, —CHF₂, —C(O)OH, —C6-, —ArC6-, and/or disulfide bond;
optionally, the first cleavable group is selected from at least one of —N₃—, —CH₂F, —C(O)NHBu, —CHF₂, —C(O)OH, —C₆—ArC₆, and/or disulfide bond.

As a possible implementation of the two-color sequencing method of the present application, the four types of reversible terminator all comprise only the first cleavable group, and the first cleavable group is —N₃— or disulfide bond.

As a possible implementation of the two-color sequencing method of the present application, the four types of reversible terminator each independently comprise the first cleavable group and/or the second cleavable group, the first cleavable group is —N₃—, and the second cleavable group is selected from groups comprising disulfide bond. As a possible implementation of the two-color sequencing method of the present application, the second cleavable group is disulfide bond.

On the basis of any one of the above embodiments, as a possible implementation of the two-color sequencing method of the present application, the first fluorescent group and the second fluorescent group are each independently selected from one of a red fluorescent group, a green fluorescent group, and a blue fluorescent group.

As a possible implementation of the two-color sequencing method of the present application, the first fluorescent group and the second fluorescent group are each independently selected from: one or combinations of more of Atto532, Atto647N, Atto390, Atto Rho6G, Alexa Fluor405, Alexa Fluor532, GM656, Alexa Fluor647, CY5, IF700, ROX, CY3, CY5 rhodamine dye, cyanine dye, coumarin dye, or phycoerythrin.

As a possible implementation of the two-color sequencing method of the present application, a combination of the first fluorescent signal and the second fluorescent signal is a combination formed of red and green; or a combination of the first fluorescent signal and the second fluorescent signal is a combination formed of red and blue.

As a possible implementation of the two-color sequencing method of the present application, the fluorescent group whose fluorescent color is green comprises Atto532, Alexa Fluor532, Atto Rho6G, or CY3;
the fluorescent group whose fluorescent color is red comprises: Atto647N, CY5, GM656, or Alexa Fluor647;
the fluorescent group whose fluorescent color is blue comprises: Atto390, Alexa Fluor405, or coumarin.

On the basis of any one of the above embodiments, as a possible implementation of the two-color sequencing method of the present application, the specific binding label is selected from at least one of biotin, digoxin, hapten, or functional derivatives thereof.

On the basis of any one of the above embodiments, as a possible implementation of the two-color sequencing method of the present application, the reversible terminators comprise at least one group shown in the following Combinations 1 to 25:
Combination 1: adenine nucleotide or derivative thereof-N3-Atto647N, guanine nucleotide or derivative thereof-N3-SS-Atto647N, thymine nucleotide or derivative thereof-N3-Biotin, and cytosine nucleotide or derivative thereof-N3-Atto532;
Combination 2: adenine nucleotide or derivative thereof-N3-CY5, guanine nucleotide or derivative thereof-N3-SS-Atto647N, thymine nucleotide or derivative thereof-N3-Biotin, and cytosine nucleotide or derivative thereof-N3-Atto532;
Combination 3: adenine nucleotide or derivative thereof-N3-CY5, guanine nucleotide or derivative thereof-N3-SS-CY5, thymine nucleotide or derivative thereof-N3-Biotin, and cytosine nucleotide or derivative thereof-N3-Atto532;
Combination 4: adenine nucleotide or derivative thereof-N3-Atto647N, guanine nucleotide or derivative thereof-N3-SS-CY5, thymine nucleotide or derivative thereof-N3-Biotin, and cytosine nucleotide or derivative thereof-N3-Atto532;
Combination 5: adenine nucleotide or derivative thereof-N3-Atto647N, guanine nucleotide or derivative thereof-N3-SS-Atto647N, thymine nucleotide or derivative thereof-N3-Atto532-Biotin, and cytosine nucleotide or derivative thereof-N3-Atto532;
Combination 6: adenine nucleotide or derivative thereof-N3-Atto647N, guanine nucleotide or derivative thereof-N3-SS-CY5, cytosine nucleotide or derivative thereof-N3-Atto532-Biotin, and cytosine nucleotide or derivative thereof-N3-Atto532;
Combination 7: adenine nucleotide or derivative thereof-N3-CY5, guanine nucleotide or derivative thereof-N3-SS-Atto647N, thymine nucleotide or derivative thereof-N3-Atto532-Biotin, and cytosine nucleotide or derivative thereof-N3-Atto532;
Combination 8: adenine nucleotide or derivative thereof-N3-CY5, guanine nucleotide or derivative thereof-N3-SS-CY5, thymine nucleotide or derivative thereof-N3-Atto532-Biotin, and cytosine nucleotide or derivative thereof-N3-Atto532;
Combination 9: adenine nucleotide or derivative thereof-N3-Atto647N, guanine nucleotide or derivative thereof-N3-SS-Atto532, thymine nucleotide or derivative thereof-N3-Biotin, and cytosine nucleotide or derivative thereof-N3-Atto532;
Combination 10: adenine nucleotide or derivative thereof-N3-CY5, guanine nucleotide or derivative thereof-N3-SS-Atto532, thymine nucleotide or derivative thereof-N3-Biotin, and cytosine nucleotide or derivative thereof-N3-Atto532;
Combination 11: adenine nucleotide or derivative thereof-N3-Atto532, guanine nucleotide or derivative thereof-N3-SS-Atto532, cytosine nucleotide or derivative thereof-N3-Biotin, and cytosine nucleotide or derivative thereof-N3-Atto647N;
Combination 12: adenine nucleotide or derivative thereof-N3-Atto532, guanine nucleotide or derivative thereof-N3-SS-Atto532, thymine nucleotide or derivative thereof-N3-Biotin, and cytosine nucleotide or derivative thereof-N3-CY5;
Combination 13: adenine nucleotide or derivative thereof-N3-Atto532, guanine nucleotide or derivative thereof-N3-SS-Atto532, cytosine nucleotide or derivative thereof-N3-Atto647N-Biotin, and cytosine nucleotide or derivative thereof-N3-Atto647N;
Combination 14: adenine nucleotide or derivative thereof-N3-Atto532, guanine nucleotide or derivative thereof-N3-SS-Atto532, thymine cytosine nucleotide or derivative thereof-N3-CY5-Biotin, and cytosine nucleotide or derivative thereof-N3-CY5;
Combination 15: adenine nucleotide or derivative thereof-N3-Atto647N, guanine nucleotide or derivative thereof-N3-Biotin, thymine nucleotide or derivative thereof-N3-Atto532-Biotin, and cytosine nucleotide or derivative thereof-N3-Atto532;
Combination 16: adenine nucleotide or derivative thereof-N3-CY5, guanine nucleotide or derivative thereof-N3-Biotin, thymine nucleotide or derivative thereof-N3-Atto532-Biotin, and cytosine nucleotide or derivative thereof-N3-Atto532;
Combination 17: adenine nucleotide or derivative thereof-N3-Atto647N, guanine nucleotide or derivative thereof-N3-Biotin, thymine cytosine nucleotide or derivative thereof-N3-Atto647N-Biotin, and cytosine nucleotide or derivative thereof-N3-Atto532;
Combination 18: adenine nucleotide or derivative thereof-N3-CY5, guanine nucleotide or derivative thereof-N3-Biotin, thymine nucleotide or derivative thereof-N3-Atto647N-Biotin, and cytosine nucleotide or derivative thereof-N3-Atto532;
Combination 19: adenine nucleotide or derivative thereof-N3-Atto647N, guanine nucleotide or derivative thereof-N3-Biotin, thymine nucleotide or derivative thereof-N3-CY5-Biotin, and cytosine nucleotide or derivative thereof-N3-Atto532;
Combination 20: adenine nucleotide or derivative thereof-N3-Atto532, guanine nucleotide or derivative thereof-N3-Biotin, thymine cytosine nucleotide or derivative thereof-N3-Atto532-Biotin, and cytosine nucleotide or derivative thereof-N3-Atto647N;
Combination 21: adenine nucleotide or derivative thereof-N3-Atto532, guanine nucleotide or derivative thereof-N3-Biotin, thymine cytosine nucleotide or derivative thereof-N3-Atto532-Biotin, and cytosine nucleotide or derivative thereof-N3-CY5;
Combination 22: adenine nucleotide or derivative thereof-N3-Atto647N, guanine nucleotide or derivative thereof-N3-Biotin, thymine cytosine nucleotide or derivative thereof-N3-Biotin, and cytosine nucleotide or derivative thereof-N3-Atto532;
Combination 23: adenine nucleotide or derivative thereof-N3-CY5, guanine nucleotide or derivative thereof-N3-Biotin, thymine nucleotide or derivative thereof-N3-Biotin, and cytosine nucleotide or derivative thereof-N3-Atto532;
Combination 24: adenine nucleotide or derivative thereof-N3-Atto532, guanine nucleotide or derivative thereof-N3-Biotin, thymine cytosine nucleotide or derivative thereof-N3-Biotin, and cytosine nucleotide or derivative thereof-N3-Atto647N;
Combination 25: adenine nucleotide or derivative thereof-N3-Atto532, guanine nucleotide or derivative thereof-N3-Biotin, thymine cytosine nucleotide or derivative thereof-N3-Biotin, and cytosine nucleotide or derivative thereof-N3-CY5.

As a possible implementation of the two-color sequencing method of the present application, in Combinations 1 to 14, the cleavable group —N3- in any one of the reversible terminators not having the specific binding label is replaced with —N3-SS—, and the cleavable group —N3-SS— in any one of the reversible terminators not having the specific binding label is replaced with —N3-; and/or
in Combinations 1 to 14, —N3-SS— is replaced with —SS—.

As a possible implementation of the two-color sequencing method of the present application, in Combinations 15 to 25, CY5 and/or Atto647N are/is replaced with GM656 or Alexa Fluor647; and/or Atto532 and/or Alexa Fluor532 are/is replaced with Atto Rho6G or CY3.

As a possible implementation of the two-color sequencing method of the present application, in Combinations 15 to 25, CY5 and/or Atto647N are/is replaced with one of the blue light dyes.

On the basis of any one of the above embodiments, as a possible implementation of the two-color sequencing method of the present application, the working concentration of the adenine nucleotide or derivative thereof is from 10 nmol/L to 700 nmol/L, the working concentration of the thymine nucleotide or derivative thereof is from 5 nmol/L to 60 nmol/L, the working concentration of the cytosine nucleotide or derivative thereof is from 10 nmol/L to 700 nmol/L, and the working concentration of the guanine nucleotide or derivative thereof is from 0.05 nmol/L to 5 nmol/L.

On the basis of any one of the above embodiments, as a possible implementation of the two-color sequencing method of the present application, the nucleic acid molecules are single-copy nucleic acid templates.

On the basis of any one of the above embodiments, as a possible implementation of the two-color sequencing method of the present application, the method further comprises:
(f) repeating (a) to (e) one or more times to determine the sequences of the template nucleic acids.

A two-color sequencing kit is provided, the two-color sequencing kit at least comprising four types of reversible terminator and a first cleavage reagent,
the four types of reversible terminator each independently comprising: a nucleotide or derivative thereof, a labeling moiety, and a first cleavable group linking the nucleotide or derivative thereof and the labeling moiety, wherein the labeling moiety is selected from a fluorescent group and/or a specific binding label, at least two of the four types of modified nucleotide carry a first fluorescent group and a second fluorescent group, which are optically distinguishable, respectively, one or two of the four types of modified nucleotide carry the specific binding label, the specific binding label is capable of specifically binding to a corresponding ligand, and the first cleavage reagent is used to cleave the first cleavable group.

On the basis of any one of the above embodiments, as a possible implementation of the two-color sequencing kit of the present application, the first cleavage reagent is an organophosphorus cleavage reagent.

On the basis of any one of the above embodiments, as a possible implementation of the two-color sequencing kit of the present application, in the organophosphorus cleavage reagent, the organophosphorus is selected from any one or more of tris(3-hydroxypropyl)phosphine, tris(2-carboxyethyl)phosphine, trimethylolphosphine, triphenylphosphine, or tris(2-hydroxyethyl)phosphine.

On the basis of any one of the above embodiments, as a possible implementation of the two-color sequencing kit of the present application, in the organophosphorus cleavage reagent, the organophosphorus is tris(3-hydroxypropyl)phosphine.

On the basis of any one of the above embodiments, as a possible implementation of the two-color sequencing kit of the present application, the working concentration of the tris(3-hydroxypropyl)phosphine is from 10 mmol/L to 100 mmol/L.

As a possible implementation of the two-color sequencing kit of the present application, three of the four types of modified nucleotide carry the first fluorescent group and the second fluorescent group, which are optically distinguishable, respectively, and the other of the four types of modified nucleotide carries the specific binding label, and one of the two types of modified nucleotide carrying the same fluorescent group comprises a second cleavable group, the second cleavable group being so located as to link the first cleavable group and the labeling moiety, and the second cleavable group having a higher cleavability than the first cleavable group, and
the kit further comprises a second cleavage reagent, the second cleavage reagent being used to cleave the second cleavable group.

On the basis of any one of the above embodiments, as a possible implementation of the two-color sequencing kit of the present application, the second cleavage reagent is a reducing agent.

On the basis of any one of the above embodiments, as a possible implementation of the two-color sequencing kit of the present application, in the reducing agent, the reducing agent is selected from DTT, glutathione, and/or β-SS-ethanol.

On the basis of any one of the above embodiments, as a possible implementation of the two-color sequencing kit of the present application, the reducing agent is glutathione.

On the basis of any one of the above embodiments, as a possible implementation of the two-color sequencing kit of the present application, the working concentration of the glutathione is from 10 mmol/L to 100 mmol/L.

On the basis of any one of the above embodiments, as a possible implementation of the two-color sequencing kit of the present application, the structures of the four types of reversible terminator satisfy one or more of the following combinations 1-4: The four reversible terminators have structures satisfying one or more of the following combinations (1)-(4):
(1) the structure of the first reversible terminator is: first nucleotide or derivative thereof-first cleavable group-first fluorescent group,
the structure of the second reversible terminator is: second nucleotide or derivative thereof-first cleavable group-specific binding label,
the structure of the third reversible terminator is: third nucleotide or derivative thereof-first cleavable group-specific binding label, and
the structure of the fourth reversible terminator is: fourth nucleotide or derivative thereof-first cleavable group-second fluorescent group;
(2) the structure of the first reversible terminator is: first nucleotide or derivative thereof-first cleavable group-first fluorescent group,
the structure of the second reversible terminator is: second nucleotide or derivative thereof-first cleavable group-specific binding label,
the structure of the third reversible terminator is: third nucleotide or derivative thereof-first cleavable group-specific binding label, and
the structure of the fourth reversible terminator is: fourth nucleotide or derivative thereof-first cleavable group-second fluorescent group-specific binding label;
(3) the structure of the first reversible terminator is: first nucleotide or derivative thereof-first cleavable group-first fluorescent group,
the structure of the second reversible terminator is: second nucleotide or derivative thereof-first cleavable group-specific binding label,
the structure of the third reversible terminator is: third nucleotide or derivative thereof-first cleavable group-second fluorescent group, and
the structure of the fourth reversible terminator is: fourth nucleotide or derivative thereof-first cleavable group-second cleavable group-first fluorescent group-specific binding label;
(4) the structure of the first reversible terminator is: first nucleotide or derivative thereof-first cleavable group-first fluorescent group,
the structure of the second reversible terminator is: second nucleotide or derivative thereof-first cleavable group-second fluorescent group-specific binding label,
the structure of the third reversible terminator is: third nucleotide or derivative thereof-first cleavable group-second fluorescent group, and
the structure of the fourth reversible terminator is: fourth nucleotide or derivative thereof-first cleavable group-second cleavable group-first fluorescent group;
wherein in each combination, the first nucleotide or derivative thereof, the second nucleotide or derivative thereof, the third nucleotide or derivative thereof, and the fourth nucleotide or derivative thereof are selected from one of adenine or derivative thereof, thymine or derivative thereof, cytosine or derivative thereof, or guanine or derivative thereof, and the first nucleotide or derivative thereof, the second nucleotide or derivative thereof, the third nucleotide or derivative thereof, and the fourth nucleotide or derivative thereof are different from each other.

On the basis of any one of the above embodiments, as a possible implementation of the two-color sequencing kit of the present application, the first cleavable group is selected from groups comprising at least one of —N3-, —CH₂F, —C(O)NHBu, —CHF₂, —C(O)OH, —C6-, —ArC6-, and/or disulfide bond.

On the basis of any one of the above embodiments, as a possible implementation of the two-color sequencing kit of the present application, the first cleavable group is selected from at least one of —N₃—, —CH₂F, —C(O)NHBu, —CHF₂, —C(O)OH, —C₆—ArC₆, and/or disulfide bond.

On the basis of any one of the above embodiments, as a possible implementation of the two-color sequencing kit of the present application, the four types of reversible terminator all comprise only the first cleavable group, and the first cleavable group is —N₃— or disulfide bond.

On the basis of any one of the above embodiments, as a possible implementation of the two-color sequencing kit of the present application, the four types of reversible terminator each independently comprise the first cleavable group and/or the second cleavable group, the first cleavable group is —N₃—, and the second cleavable group is selected from groups comprising disulfide bond; optionally, the second cleavable group is disulfide bond.

On the basis of any one of the above embodiments, as a possible implementation of the two-color sequencing kit of the present application, the first fluorescent group and the second fluorescent group are each independently selected from one of a red fluorescent group, a green fluorescent group, and a blue fluorescent group.

On the basis of any one of the above embodiments, as a possible implementation of the two-color sequencing kit of the present application, the first fluorescent group and the second fluorescent group are each independently selected from: one or combinations of more of Atto532, Atto647N, Atto390, Atto Rho6G, Alexa Fluor405, Alexa Fluor532, GM656, Alexa Fluor647, CY5, IF700, ROX, CY3, CY5 rhodamine dye, cyanine dye, coumarin dye, or phycoerythrin.

On the basis of any one of the above embodiments, as a possible implementation of the two-color sequencing kit of the present application, a combination of the first fluorescent signal and the second fluorescent signal is a combination formed of red and green; or a combination of the first fluorescent signal and the second fluorescent signal is a combination formed of red and blue.

On the basis of any one of the above embodiments, as a possible implementation of the two-color sequencing kit of the present application, the fluorescent group whose fluorescent color is green comprises Atto532, Alexa Fluor532, Atto Rho6G, or CY3;
the fluorescent group whose fluorescent color is red comprises: Atto647N, CY5, GM656, or Alexa Fluor647;
the fluorescent group whose fluorescent color is blue comprises: Atto390, Alexa Fluor405, or coumarin.

On the basis of any one of the above embodiments, as a possible implementation of the two-color sequencing kit of the present application, the specific binding label is selected from at least one of biotin, digoxin, hapten, or functional derivatives thereof.

On the basis of any one of the above embodiments, as a possible implementation of the two-color sequencing kit of the present application, the reversible terminators comprise at least one group shown in the following Combinations 1 to 25:
Combination 1: adenine nucleotide or derivative thereof-N3-Atto647N, guanine nucleotide or derivative thereof-N3-SS-Atto647N, thymine nucleotide or derivative thereof-N3-Biotin, and cytosine nucleotide or derivative thereof-N3-Atto532;
Combination 2: adenine nucleotide or derivative thereof-N3-CY5, guanine nucleotide or derivative thereof-N3-SS-Atto647N, thymine nucleotide or derivative thereof-N3-Biotin, and cytosine nucleotide or derivative thereof-N3-Atto532;
Combination 3: adenine nucleotide or derivative thereof-N3-CY5, guanine nucleotide or derivative thereof-N3-SS-CY5, thymine nucleotide or derivative thereof-N3-Biotin, and cytosine nucleotide or derivative thereof-N3-Atto532;
Combination 4: adenine nucleotide or derivative thereof-N3-Atto647N, guanine nucleotide or derivative thereof-N3-SS-CY5, thymine nucleotide or derivative thereof-N3-Biotin, and cytosine nucleotide or derivative thereof-N3-Atto532;
Combination 5: adenine nucleotide or derivative thereof-N3-Atto647N, guanine nucleotide or derivative thereof-N3-SS-Atto647N, thymine nucleotide or derivative thereof-N3-Atto532-Biotin, and cytosine nucleotide or derivative thereof-N3-Atto532;
Combination 6: adenine nucleotide or derivative thereof-N3-Atto647N, guanine nucleotide or derivative thereof-N3-SS-CY5, cytosine nucleotide or derivative thereof-N3-Atto532-Biotin, and cytosine nucleotide or derivative thereof-N3-Atto532;
Combination 7: adenine nucleotide or derivative thereof-N3-CY5, guanine nucleotide or derivative thereof-N3-SS-Atto647N, thymine nucleotide or derivative thereof-N3-Atto532-Biotin, and cytosine nucleotide or derivative thereof-N3-Atto532;
Combination 8: adenine nucleotide or derivative thereof-N3-CY5, guanine nucleotide or derivative thereof-N3-SS-CY5, thymine nucleotide or derivative thereof-N3-Atto532-Biotin, and cytosine nucleotide or derivative thereof-N3-Atto532;
Combination 9: adenine nucleotide or derivative thereof-N3-Atto647N, guanine nucleotide or derivative thereof-N3-SS-Atto532, thymine nucleotide or derivative thereof-N3-Biotin, and cytosine nucleotide or derivative thereof-N3-Atto532;
Combination 10: adenine nucleotide or derivative thereof-N3-CY5, guanine nucleotide or derivative thereof-N3-SS-Atto532, thymine nucleotide or derivative thereof-N3-Biotin, and cytosine nucleotide or derivative thereof-N3-Atto532;
Combination 11: adenine nucleotide or derivative thereof-N3-Atto532, guanine nucleotide or derivative thereof-N3-SS-Atto532, cytosine nucleotide or derivative thereof-N3-Biotin, and cytosine nucleotide or derivative thereof-N3-Atto647N;
Combination 12: adenine nucleotide or derivative thereof-N3-Atto532, guanine nucleotide or derivative thereof-N3-SS-Atto532, thymine nucleotide or derivative thereof-N3-Biotin, and cytosine nucleotide or derivative thereof-N3-CY5;
Combination 13: adenine nucleotide or derivative thereof-N3-Atto532, guanine nucleotide or derivative thereof-N3-SS-Atto532, cytosine nucleotide or derivative thereof-N3-Atto647N-Biotin, and cytosine nucleotide or derivative thereof-N3-Atto647N;
Combination 14: adenine nucleotide or derivative thereof-N3-Atto532, guanine nucleotide or derivative thereof-N3-SS-Atto532, thymine cytosine nucleotide or derivative thereof-N3-CY5-Biotin, and cytosine nucleotide or derivative thereof-N3-CY5;
Combination 15: adenine nucleotide or derivative thereof-N3-Atto647N, guanine nucleotide or derivative thereof-N3-Biotin, thymine nucleotide or derivative thereof-N3-Atto532-Biotin, and cytosine nucleotide or derivative thereof-N3-Atto532;
Combination 16: adenine nucleotide or derivative thereof-N3-CY5, guanine nucleotide or derivative thereof-N3-Biotin, thymine nucleotide or derivative thereof-N3-Atto532-Biotin, and cytosine nucleotide or derivative thereof-N3-Atto532;
Combination 17: adenine nucleotide or derivative thereof-N3-Atto647N, guanine nucleotide or derivative thereof-N3-Biotin, thymine cytosine nucleotide or derivative thereof-N3-Atto647N-Biotin, and cytosine nucleotide or derivative thereof-N3-Atto532;
Combination 18: adenine nucleotide or derivative thereof-N3-CY5, guanine nucleotide or derivative thereof-N3-Biotin, thymine nucleotide or derivative thereof-N3-Atto647N-Biotin, and cytosine nucleotide or derivative thereof-N3-Atto532;
Combination 19: adenine nucleotide or derivative thereof-N3-Atto647N, guanine nucleotide or derivative thereof-N3-Biotin, thymine nucleotide or derivative thereof-N3-CY5-Biotin, and cytosine nucleotide or derivative thereof-N3-Atto532;
Combination 20: adenine nucleotide or derivative thereof-N3-Atto532, guanine nucleotide or derivative thereof-N3-Biotin, thymine cytosine nucleotide or derivative thereof-N3-Atto532-Biotin, and cytosine nucleotide or derivative thereof-N3-Atto647N;
Combination 21: adenine nucleotide or derivative thereof-N3-Atto532, guanine nucleotide or derivative thereof-N3-Biotin, thymine cytosine nucleotide or derivative thereof-N3-Atto532-Biotin, and cytosine nucleotide or derivative thereof-N3-CY5;
Combination 22: adenine nucleotide or derivative thereof-N3-Atto647N, guanine nucleotide or derivative thereof-N3-Biotin, thymine cytosine nucleotide or derivative thereof-N3-Biotin, and cytosine nucleotide or derivative thereof-N3-Atto532;
Combination 23: adenine nucleotide or derivative thereof-N3-CY5, guanine nucleotide or derivative thereof-N3-Biotin, thymine nucleotide or derivative thereof-N3-Biotin, and cytosine nucleotide or derivative thereof-N3-Atto532;
Combination 24: adenine nucleotide or derivative thereof-N3-Atto532, guanine nucleotide or derivative thereof-N3-Biotin, thymine cytosine nucleotide or derivative thereof-N3-Biotin, and cytosine nucleotide or derivative thereof-N3-Atto647N;
Combination 25: adenine nucleotide or derivative thereof-N3-Atto532, guanine nucleotide or derivative thereof-N3-Biotin, thymine cytosine nucleotide or derivative thereof-N3-Biotin, and cytosine nucleotide or derivative thereof-N3-CY5.

On the basis of any one of the above embodiments, as a possible implementation of the two-color sequencing kit of the present application, in Combinations 1 to 14, the cleavable group —N3- in any one of the reversible terminators not having the specific binding label is replaced with —N3-SS—, and the cleavable group —N3-SS— in any one of the reversible terminators not having the specific binding label is replaced with —N3-; and/or
in Combinations 1 to 14, —N3-SS— is replaced with —SS—.

On the basis of any one of the above embodiments, as a possible implementation of the two-color sequencing kit of the present application, in Combinations 15 to 25, CY5 and/or Atto647N are/is replaced with GM656 or Alexa Fluor647; and/or Atto532 and/or Alexa Fluor532 are/is replaced with Atto Rho6G or CY3.

As a possible implementation of the two-color sequencing method of the present application, in Combinations 15 to 25, CY5 and/or Atto647N are/is replaced with one of the blue light dyes.

On the basis of any one of the above embodiments, as a possible implementation of the two-color sequencing kit of the present application, the working concentration of the adenine nucleotide or derivative thereof is from 10 nmol/L to 700 nmol/L, the working concentration of the thymine nucleotide or derivative thereof is from 5 nmol/L to 60 nmol/L, the working concentration of the cytosine nucleotide or derivative thereof is from 10 nmol/L to 700 nmol/L, and the working concentration of the guanine nucleotide or derivative thereof is from 0.05 nmol/L to 5 nmol/L.

The present disclosure provides a two-color sequencing method, comprising the steps of:
step 1, providing four types of reversible terminator: a first reversible terminator, a second reversible terminator, a third reversible terminator, and a fourth reversible terminator, the four types of reversible terminator each independently comprising: a nucleotide or derivative thereof, a labeling moiety, and a first cleavable group and/or a second cleavable group linking the nucleotide or derivative thereof and the labeling moiety, the nucleotide or derivative thereof being selected from adenine or derivative thereof, thymine or derivative thereof, cytosine or derivative thereof, or guanine or derivative thereof,
step 2, allowing the reversible terminators to bind to and extend along nucleic acid template-primer complexes via polymerase chain reactions to obtain extension products;
step 3, exciting the extension products and collecting fluorescent signals therefrom to obtain a first image, the first image comprising a first signal, a second signal, a third signal, and a fourth signal from the first reversible terminator, the second reversible terminator, the third reversible terminator, and the fourth reversible terminator, respectively;
step 4, performing a first reaction and a second reaction on the extension products, or performing a second reaction on the extension products, to obtain reaction products;
step 5, exciting the reaction products and collecting fluorescent signals therefrom to obtain a second image, the second image comprising a fifth signal, a sixth signal, a seventh signal, and an eighth signal from the first reversible terminator, the second reversible terminator, the third reversible terminator, and the fourth reversible terminator, respectively;
step 6, determining the type of the reversible terminators bound to the nucleic acid template-primer complexes according to the optical signals of the first image and the second image;
step 7, performing a third reaction on the reaction products; and
step 8, repeating step 2 to step 7 one or more times to determine the sequence of the template nucleic acids;
wherein,
the labeling moiety is selected from: at least one of a combination of a fluorescent group and a specific binding label, the fluorescent group, and the specific binding label, the labeling substance comprising a ligand and a fluorescent group bound to the ligand;
when the reversible terminator comprises both the first cleavable group and the second cleavable group, the first cleavable group and the second cleavable group are different, and the second cleavable group has a higher cleavability than the first cleavable group;
the first reaction is a reaction in which the second cleavable group is cleaved;
the second reaction is a binding reaction between the ligand and the specific binding label;
the third reaction is a reaction in which the first cleavable group or the second cleavable group is cleaved;
the first signal, the second signal, the third signal, the fourth signal, the fifth signal, the sixth signal, the seventh signal, and the eighth signal are each independently one of a first bright spot, a second bright spot, a third bright spot, and no bright spot, wherein the first bright spot indicates a first fluorescent color feature, the second bright spot indicates a second fluorescent color feature, and the third bright spot indicates both the first fluorescent color feature and the second fluorescent color feature,
and the first signal, the second signal, the third signal, the fourth signal, the fifth signal, the sixth signal, the seventh signal, and the eighth signal satisfy the following conditions a) to f) at the same time:
a) the first signal and the fifth signal are the same,
b) the fourth signal and the eighth signal are the same,
c) the first signal and the fourth signal are different,
d) the second signal and the sixth signal are different,
e) the third signal and the seventh signal are different,
f) the sixth signal and the seventh signal are different and/or the second signal and the third signal are different.

As one of the embodiments of the two-color sequencing method of the present disclosure. On the basis of any one of the above embodiments, as a possible implementation of the two-color sequencing kit of the present application, the labeling moiety comprises the fluorescent group and the specific binding label, and the fluorescent group in the labeling moiety and the fluorescent group in the labeling substance are selected from the first fluorescent group and the second fluorescent group, which are distinguishable in color.

On the basis of any one of the above embodiments, as a possible implementation of the two-color sequencing kit of the present application, among the reversible terminators having the same cleavable group, there are at least two types of reversible terminator whose labeling moieties have different fluorescent colors.

On the basis of any one of the above embodiments, as a possible implementation of the two-color sequencing kit of the present application, the first cleavable group is selected from groups comprising at least one of —N3-, —CH2F, —C(O)NHBu, —CHF2, —C(O)OH, —C6-, —ArC6-, and/or disulfide bond.

On the basis of any one of the above embodiments, as a possible implementation of the two-color sequencing kit of the present application, the first cleavable group is selected from at least one of —N₃—, —CH₂F, —C(O)NHBu, —CHF₂, —C(O)OH, —C₆, —ArC₆, and/or disulfide bond.

On the basis of any one of the above embodiments, as a possible implementation of the two-color sequencing kit of the present application, the first cleavable group is —N3- or disulfide bond.

On the basis of any one of the above embodiments, as a possible implementation of the two-color sequencing kit of the present application, the second cleavable group is selected from groups comprising disulfide bond.

On the basis of any one of the above embodiments, as a possible implementation of the two-color sequencing kit of the present application, the second cleavable group is disulfide bond.

On the basis of any one of the above embodiments, as a possible implementation of the two-color sequencing kit of the present application, the fluorescent group is selected from the first fluorescent group and the second fluorescent group, which are distinguishable in color.

On the basis of any one of the above embodiments, as a possible implementation of the two-color sequencing kit of the present application, in the first signal to the eighth signal,
the first signal, the second signal, and the fifth signal are the same, the fourth signal and the eighth signal are the same, the first signal and the fourth signal are different, the second signal and the third signal are different, the second signal, the fourth signal, and the sixth signal are different from each other, and the third signal and the seventh signal are different from each other; specifically, see Table 5, 4, 2, or 1;
or, the first signal and the fifth signal are the same, the fourth signal and the eighth signal are the same, the first signal and the fourth signal are different, the second signal, the first signal, and the third signal are different from each other, and the third signal, the fourth signal, and the seventh signal are different from each other; specifically, see Table 3;
or, the first signal, the fifth signal, and the sixth signal are the same, the third signal, the fourth signal, and the eighth signal are the same, the first signal and the third signal are different, the second signal is different from the first signal and the third signal, respectively, and the seventh signal and the third signal are different; specifically, see Table 6;
or, the first signal and the fifth signal are the same, the fourth signal and the eighth signal are the same, the first signal and the fourth signal are different, the second signal and the third signal are the same, the second signal is different from the sixth signal and the seventh signal, respectively, and the seventh signal is different from the third signal and the sixth signal, respectively; specifically, see Table 9 or 10;
or, the first signal, the third signal, and the fifth signal are the same, the fourth signal and the eighth signal are the same, the first signal and the fourth signal are different, the second signal and the third signal are different, the second signal, the first signal, and the fourth signal are different from each other, and the third signal, the fourth signal, and the seventh signal are different from each other; specifically, see Table 7 or 8;
It should be understood that the scenarios to which the above word “optionally” corresponds may each occur independently and separately in different implementations, and of course, they may also be combined arbitrarily to form different implementations.

On the basis of the above embodiments, further optionally,
the first signal and the fifth signal are both selected from one of the first bright spot or the second bright spot, the fourth signal and the eighth signal are both selected from one of the first bright spot or the second bright spot (specifically, see Tables 1 to 10); and the first signal, the second signal, the third signal, the sixth signal, and the seventh signal satisfy one of the following conditions 1) to 4):
1) the second signal is selected from the first bright spot or the second bright spot, the third signal is selected from the first bright spot or the second bright spot, the sixth signal is the no bright spot, and the seventh signal is the third bright spot; specifically, see Table 2 or 5.
2) the second signal is selected from the first bright spot or the second bright spot, the third signal is the no bright spot, the sixth signal is the no bright spot, and the seventh signal is one of the first bright spot or the second bright spot; specifically, see Table 1, 3 or 4;
3) the second signal is the no bright spot, the third signal is selected from the first bright spot or the second bright spot, the sixth signal is one of the first bright spot or the second bright spot, and the seventh signal is the third bright spot; specifically, see Table 6, 7, or 8; and
4) the second signal is no bright spot, the third signal is no bright spot, the sixth signal is one of the first bright spot or the second bright spot, and the seventh signal is the third bright spot; specifically, see Table 9 or 10.

It should be understood that the scenarios to which the above word “optionally” corresponds may each occur independently and separately in different implementations, and of course, they may also be combined arbitrarily to form different implementations.

In view of the above embodiments, the two-color sequencing method of the present disclosure comprises different implementations, and two implementations will be explained below. For convenience of description, the two implementations are designated Scheme 1 and Scheme 2, respectively.

In Scheme 1, one or more of the four types of reversible terminator comprise(s) the first cleavable group, and one or more of the four types of reversible terminator comprise(s) the second cleavable group; that is, the four types of reversible terminator comprise two types of cleavable group, and in step 4, a first reaction and a second reaction are performed on the extension products to obtain reaction products.

On the basis of any one of the above embodiments, as a possible implementation of the two-color sequencing kit of the present application, the first signal, the second signal, the third signal, the sixth signal, and the seventh signal satisfy condition 1) or condition 2), and the structures of the four types of reversible terminator satisfy the following:
the structure of the first reversible terminator is: first nucleotide or derivative thereof-first cleavable group-first fluorescent group;
the structure of the second reversible terminator is one of the following scenarios: second nucleotide or derivative thereof-first cleavable group-second cleavable group-first fluorescent group or second fluorescent group; or second nucleotide or derivative thereof-second cleavable group-first fluorescent group or second fluorescent group;
the structure of the third reversible terminator is one of the following scenarios: second nucleotide or derivative thereof-first cleavable group-specific binding label; or third nucleotide or derivative thereof-first cleavable group-first fluorescent group or second fluorescent group-specific binding label,
the structure of the fourth reversible terminator is: fourth nucleotide or derivative thereof-first cleavable group-second fluorescent group;
the second reversible terminator and the third reversible terminator do not comprise the specific binding label at the same time;
wherein the first nucleotide or derivative thereof, the second nucleotide or derivative thereof, the third nucleotide or derivative thereof, and the fourth nucleotide or derivative thereof are selected from one of adenine or derivative thereof, thymine or derivative thereof, cytosine or derivative thereof, or guanine or derivative thereof, and the first nucleotide or derivative thereof, the second nucleotide or derivative thereof, the third nucleotide or derivative thereof, and the fourth nucleotide or derivative thereof are different from each other.

On the basis of any one of the above embodiments, as a possible implementation of the two-color sequencing kit of the present application, the first fluorescent group and the second fluorescent group are each independently selected from: one or combinations of more of Atto532, Atto647N, Atto390, Atto Rho6G, Alexa Fluor405, Alexa Fluor532, GM656, Alexa Fluor647, CY5, IF700, ROX, CY3, CY5 rhodamine dye, cyanine dye, coumarin dye, and/or phycoerythrin.

On the basis of any one of the above embodiments, as a possible implementation of the two-color sequencing kit of the present application, the fluorescent color of the first fluorescent group is red, green, or blue;
the fluorescent color of the second fluorescent group is red, green, or blue.

On the basis of any one of the above embodiments, as a possible implementation of the two-color sequencing kit of the present application, a combination of the first fluorescent signal and the second fluorescent signal is a combination formed of red and green; or a combination of the first fluorescent signal and the second fluorescent signal is a combination formed of red and blue.

On the basis of any one of the above embodiments, as a possible implementation of the two-color sequencing kit of the present application, the fluorescent group whose fluorescent color is green comprises Atto532, Alexa Fluor532, Atto Rho6G, or CY3;
the fluorescent group whose fluorescent color is red comprises: Atto647N, CY5, GM656, or Alexa Fluor647;
the fluorescent group whose fluorescent color is blue comprises: Atto390, Alexa Fluor405, or coumarin.

On the basis of any one of the above embodiments, as a possible implementation of the two-color sequencing kit of the present application, the labeling substance comprises n fluorescent groups, wherein n is 1 or 2.

On the basis of any one of the above embodiments, as a possible implementation of the two-color sequencing kit of the present application, a combination of the specific binding label and the ligand is selected from at least one of streptavidin (SA)-Biotin combination, Biotin-streptavidin (SA) combination, Biotin-avidin combination, or antibody-antigen combination.

On the basis of any one of the above embodiments, as a possible implementation of the two-color sequencing kit of the present application, a combination of specific binding label and the ligand is selected from Biotin-streptavidin (SA) combination.

Below, for convenience of understanding and description, the first cleavable group is exemplified by —N3- and the second cleavable group is exemplified by disulfide bond —SS— to explain Scheme 1 in conjunction with specific examples. However, it should be understood that the combination of the first cleavable group and the second cleavable group is not limited to the above scenario, and other cleavable groups listed in the present disclosure may be selected to form combinations. In addition, the nucleotides or derivatives thereof provided by the present disclosure may be deoxyribonucleotides or derivatives thereof, or may be ribonucleotides or derivatives thereof. For convenience of description, the following exemplifications are uniformly explained exemplified by deoxyribonucleotides or derivatives thereof. Specifically, adenine deoxyribonucleotide or derivative thereof is represented by dA, guanine deoxyribonucleotidesor derivative thereof is represented by dG, cytosine deoxyribonucleotide or derivative thereof is represented by dC, and thymine deoxyribonucleotide or derivative thereof is represented by dT.

In Scheme 1 of two-color sequencing of the present disclosure, exemplarily, the steps and signal situations of two-color sequencing may be Scenario 1a as shown in Table 1 below:

TABLE 1
Signal changes and detection steps associated with two-color sequencing method 1a

| Reversible | | | | | |
|---|---|---|---|---|---|
| terminators | First image | Cleavage | Binding | Second image | Cleavage |
| first nucleotide or | 1 first signal | | | 1 fifth signal red | N3 |
| derivative thereof- | red | | | | |
| N3-red fluorescent | | | | | |
| group | | | | | |
| second nucleotide or | 1 second | SS | | 0 sixth signal | N3 |
| derivative thereof- | signal red | | | | |
| N3-SS-red | | | | | |
| fluorescent group, or | | | | | |
| second nucleotide or | | | | | |
| derivative thereof- | | | | | |
| SS-red fluorescent | | | | | |
| group | | | | | |
| third nucleotide or | 0 third signal | | ligand-red | 1 seventh signal | N3 |
| derivative thereof- | | | fluorescent | red | |
| N3-specific binding | | | group | | |
| label | | | | | |
| fourth nucleotide or | 1 fourth signal | | | 1 eighth signal | N3 |
| derivative thereof- | green | | | green | |
| N3-green fluorescent | | | | | |
| group | | | | | |

It should be understood that the types of fluorescent group in the reversible terminators provided in Table 1 above may be exchanged for one another, and in this instance, the combination of reversible terminators may also be as follows:
first nucleotide or derivative thereof-N3-green fluorescent group, second nucleotide or derivative thereof-N3-SS-green fluorescent group (or second nucleotide or derivative thereof-SS-green fluorescent group), third nucleotide or derivative thereof-N3-specific binding label, and fourth nucleotide or derivative thereof-N3-red fluorescent group. In this instance, the labeling substance for the third reversible terminator is correspondingly: ligand-green fluorescent group.

Exemplarily, the combination of reversible terminators that can realize the steps and signal situations of Scenario 1a as shown in Table 1 is selected from at least one of the following Combination 1 to Combination 4:
Combination 1: adenine nucleotide or derivative thereof-N3-Atto647N, guanine nucleotide or derivative thereof-N3-SS-Atto647N, thymine nucleotide or derivative thereof-N3-Biotin, and cytosine nucleotide or derivative thereof-N3-Atto532;
Combination 2: adenine nucleotide or derivative thereof-N3-CY5, guanine nucleotide or derivative thereof-N3-SS-Atto647N, thymine nucleotide or derivative thereof-N3-Biotin, and cytosine nucleotide or derivative thereof-N3-Atto532;
Combination 3: adenine nucleotide or derivative thereof-N3-CY5, guanine nucleotide or derivative thereof-N3-SS-CY5, thymine nucleotide or derivative thereof-N3-Biotin, and cytosine nucleotide or derivative thereof-N3-Atto532;
Combination 4: adenine nucleotide or derivative thereof-N3-Atto647N, guanine nucleotide or derivative thereof-N3-SS-CY5, thymine nucleotide or derivative thereof-N3-Biotin, and cytosine nucleotide or derivative thereof-N3-Atto532.

In Scheme 1 of two-color sequencing of the present disclosure, exemplarily, the steps and signal situations of two-color sequencing may also be Scenario 1b as shown in Table 2 below:

TABLE 2
Signal changes and detection steps associated with two-color sequencing method 1b

| Reversible terminators | First image | Cleavage | Binding | Second image | Cleavage |
|---|---|---|---|---|---|
| first nucleotide or | 1 first signal | | | 1 fifth signal | N3 |
| derivative thereof-N3- | red | | | red | |
| red fluorescent group | | | | | |
| second nucleotide or | 1 second signal | SS | | 0 sixth signal | N3 |
| derivative thereof-N3- | red | | | | |
| SS-red fluorescent | | | | | |
| group, or second | | | | | |
| nucleotide or derivative | | | | | |
| thereof-SS-red | | | | | |
| fluorescent group | | | | | |
| third nucleotide or | 1 third signal | | ligand- | 1 seventh | N3 |
| derivative thereof-N3- | green | | red | signal red | |
| green fluorescent | | | fluorescent | green | |
| group-specific binding | | | group | | |
| label | | | | | |
| fourth nucleotide or | 1 fourth signal | | | 1 eighth signal | N3 |
| derivative thereof-N3- | green | | | green | |
| green fluorescent group | | | | | |

It should be understood that the types of fluorescent group in the reversible terminators provided in Table 2 above may be exchanged for one another, and in this instance, the combination of reversible terminators may also be as follows:
first nucleotide or derivative thereof-N3-green fluorescent group, second nucleotide or derivative thereof-N3-SS-green fluorescent group (or second nucleotide or derivative thereof-SS-green fluorescent group), third nucleotide or derivative thereof-N3-red fluorescent group-specific binding label, and fourth nucleotide or derivative thereof-N3-red fluorescent group. In this instance, the labeling substance for the third reversible terminator is correspondingly: ligand-green fluorescent group.

Exemplarily, the combination of reversible terminators that can realize the steps and signal situations of Scenario 1b as shown in Table 2 is selected from at least one of the following Combination 5 to Combination 8:
Combination 5: adenine nucleotide or derivative thereof-N3-Atto647N, guanine nucleotide or derivative thereof-N3-SS-Atto647N, thymine nucleotide or derivative thereof-N3-Atto532-Biotin, and cytosine nucleotide or derivative thereof-N3-Atto532;
Combination 6: adenine nucleotide or derivative thereof-N3-Atto647N, guanine nucleotide or derivative thereof-N3-SS-CY5, thymine nucleotide or derivative thereof-N3-Atto532-Biotin, and cytosine nucleotide or derivative thereof-N3-Atto532;
Combination 7: adenine nucleotide or derivative thereof-N3-CY5, guanine nucleotide or derivative thereof-N3-SS-Atto647N, thymine nucleotide or derivative thereof-N3-Atto532-Biotin, and cytosine nucleotide or derivative thereof-N3-Atto532;
Combination 8: adenine nucleotide or derivative thereof-N3-CY5, guanine nucleotide or derivative thereof-N3-SS-CY5, thymine nucleotide or derivative thereof-N3-Atto532 thereof-Biotin, and cytosine nucleotide or derivative thereof-N3-Atto532 thereof.

In Scheme 1 of two-color sequencing of the present disclosure, exemplarily, the steps and signal situations of two-color sequencing may be Scenario 1c as shown in Table 2 below:

TABLE 3
Signal changes and detection steps associated with two-color sequencing method 1c

| Reversible terminators | First image | Cleavage | Binding | Second image | Cleavage |
|---|---|---|---|---|---|
| first nucleotide or | 1 first signal | | | 1 fifth signal | N3 |
| derivative thereof-N3-red | red | | | red | |
| fluorescent group | | | | | |
| second nucleotide or | 1 second | SS | | 0 sixth signal | N3 |
| derivative thereof-N3-SS- | signal green | | | | |
| green fluorescent group, | | | | | |
| or second nucleotide or | | | | | |
| derivative thereof-SS- | | | | | |
| green fluorescent group” | | | | | |
| third nucleotide or | 0 third | | ligand-red | 1 seventh | N3 |
| derivative thereof-N3- | signal | | fluorescent | signal red | |
| specific binding label | | | group | | |
| fourth nucleotide or | 1 fourth | | | 1 eighth | N3 |
| derivative thereof-N3- | signal green | | | signal green | |
| green fluorescent group | | | | | |

It should be understood that the types of fluorescent group in the reversible terminators provided in Table 3 above may be exchanged for one another, and in this instance, the combination of reversible terminators may also be as follows:
first nucleotide or derivative thereof-N3-green fluorescent group, second nucleotide or derivative thereof-N3-SS-red fluorescent group (or second nucleotide or derivative thereof-SS-red fluorescent group), third nucleotide or derivative thereof-N3-specific binding label, and fourth nucleotide or derivative thereof-N3-red fluorescent group. In this instance, the labeling substance for the third reversible terminator is correspondingly: ligand-green fluorescent group.

Exemplarily, the combination of reversible terminators that can realize the steps and signal situations of Scenario 1c as shown in Table 3 is selected from at least one of the following Combination 9 or Combination 10:
Combination 9: adenine nucleotide or derivative thereof-N3-Atto647N, guanine nucleotide or derivative thereof-N3-SS-Atto532, thymine nucleotide or derivative thereof-N3-Biotin, and cytosine nucleotide or derivative thereof-N3-Atto532;
Combination 10: adenine nucleotide or derivative thereof-N3-CY5, guanine nucleotide or derivative thereof-N3-SS-Atto532, thymine nucleotide or derivative thereof-N3-Biotin, and cytosine nucleotide or derivative thereof-N3-Atto532;
In Scheme 1 of two-color sequencing of the present disclosure, exemplarily, the steps and signal situations of two-color sequencing may be Scenario 1d as shown in Table 4 below:

TABLE 4
Signal changes and detection steps associated with two-color sequencing method 1d

| Reversible terminators | First image | Cleavage | Binding | Second image | Cleavage |
|---|---|---|---|---|---|
| first nucleotide or | 1 first signal | | | 1 fifth signal | N3 |
| derivative thereof-N3- | green | | | green | |
| green fluorescent | | | | | |
| group | | | | | |
| second nucleotide or | 1 second | SS | | 0 sixth signal | N3 |
| derivative thereof-N3- | signal green | | | | |
| SS-green fluorescent | | | | | |
| group, or second | | | | | |
| nucleotide or | | | | | |
| derivative thereof-SS- | | | | | |
| green fluorescent | | | | | |
| group | | | | | |
| third nucleotide or | 0 third signal | | ligand- | 1 seventh | N3 |
| derivative thereof-N3- | | | green | signal green | |
| specific binding label | | | fluorescent | | |
| | | | group | | |
| fourth nucleotide or | 1 fourth | | | 1 eighth signal | N3 |
| derivative thereof-N3- | signal red | | | red | |
| red fluorescent group | | | | | |

It should be understood that the types of fluorescent group in the reversible terminators provided in Table 4 above may be exchanged for one another, and in this instance, the combination of reversible terminators may also be as follows:
first nucleotide or derivative thereof-N3-red fluorescent group, second nucleotide or derivative thereof-N3-SS-red fluorescent group (or second nucleotide or derivative thereof-SS-red fluorescent group), third nucleotide or derivative thereof-N3-specific binding label, and fourth nucleotide or derivative thereof-N3-green fluorescent group. In this instance, the labeling substance for the third reversible terminator is correspondingly: ligand-red fluorescent group.

Exemplarily, the combination of reversible terminators that can realize the steps and signal situations of Scenario 1d as shown in Table 4 is selected from at least one of the following Combination 11 or Combination 12:
Combination 11: adenine nucleotide or derivative thereof-N3-Atto532, guanine nucleotide or derivative thereof-N3-SS-Atto532, cytosine nucleotide or derivative thereof-N3-Biotin, and cytosine nucleotide or derivative thereof-N3-Atto647N;
Combination 12: adenine nucleotide or derivative thereof-N3-Atto532, guanine nucleotide or derivative thereof-N3-SS-Atto532, thymine nucleotide or derivative thereof-N3-Biotin, and cytosine nucleotide or derivative thereof-N3-CY5.

In Scheme 1 of two-color sequencing of the present disclosure, exemplarily, the steps and signal situations of two-color sequencing may be Scenario 1e as shown in Table 5 below:

TABLE 5
Signal changes and detection steps associated with two-color sequencing method 1e

| Reversible terminators | First image | Cleavage | Binding | Second image | Cleavage |
|---|---|---|---|---|---|
| first nucleotide or | 1 first signal | | | 1 fifth signal | N3 |
| derivative thereof-N3- | green | | | green | |
| green fluorescent | | | | | |
| group | | | | | |
| second nucleotide or | 1 second | SS | | 0 sixth signal | N3 |
| derivative thereof-N3- | signal green | | | | |
| SS-green fluorescent | | | | | |
| group, or second | | | | | |
| nucleotide or | | | | | |
| derivative thereof-SS- | | | | | |
| green fluorescent | | | | | |
| group | | | | | |
| third nucleotide or | 1 third signal | | SA- | 1 seventh signal | N3 |
| derivative thereof-N3- | red | | Atto532 | red green | |
| red fluorescent group- | | | | | |
| specific binding label | | | | | |
| fourth nucleotide or | 1 fourth | | | 1 eighth signal | N3 |
| derivative thereof-N3- | signal red | | | red | |
| red fluorescent group | | | | | |

It should be understood that the types of fluorescent group in the reversible terminators provided in Table 5 above may be exchanged for one another, and in this instance, the combination of reversible terminators may also be as follows:
first nucleotide or derivative thereof-N3-green fluorescent group, second nucleotide or derivative thereof-N3-SS-green fluorescent group (or second nucleotide or derivative thereof-SS-green fluorescent group), third nucleotide or derivative thereof-N3-red fluorescent group-specific binding label, and fourth nucleotide or derivative thereof-N3-red fluorescent group. In this instance, the labeling substance for the third reversible terminator is correspondingly: ligand-green fluorescent group.

Exemplarily, the combination of reversible terminators that can realize the steps and signal situations of Scenario 1e as shown in Table 5 is selected from at least one of the following Combination 13 or Combination 14:
Combination 13: adenine nucleotide or derivative thereof-N3-Atto532, guanine nucleotide or derivative thereof-N3-SS-Atto532, cytosine nucleotide or derivative thereof-N3-Atto647N-Biotin, and cytosine nucleotide or derivative thereof-N3-Atto647N;
Combination 14: adenine nucleotide or derivative thereof-N3-Atto532, guanine nucleotide or derivative thereof-N3-SS-Atto532, thymine cytosine nucleotide or derivative thereof-N3-CY5-Biotin, and cytosine nucleotide or derivative thereof-N3-CY5.

As another implementation for realizing the two-color sequencing of the present disclosure, in Combinations 1 to 14, the cleavable group —N3- in any one of the reversible terminators not having the specific binding label is replaced with —N3-SS—, and the cleavable group —N3-SS— in any one of the reversible terminators not having the specific binding label is replaced with —N3-; and/or
In Combinations 1 to 14, —N3-SS— is replaced with —SS—.

In embodiments of Scheme 1, replacement of the fluorescent groups may be performed on Combinations 1 to 14 to form new scenarios. Exemplarily, in some embodiments, in Combinations 1 to 14, CY5 and/or Atto647N are/is replaced with GM656 or Alexa Fluor647, and/or Atto532 and/or Alexa Fluor532 are/is replaced with Atto Rho6G or CY3;
in other embodiments, in Combinations 1 to 14, CY5 and/or Atto647N are/is replaced with one of the blue light dyes.

Replacement of the fluorescent groups described in the present disclosure can occur before replacement of the cleavable groups, or can occur after replacement of the cleavable groups. Performing replacement of colors before or after replacement of the cleavable groups can form new combinations of reversible terminators suitable for two-color sequencing, to be used for detection.

In specific examples of the present disclosure, in Scheme 1 of the two-color sequencing method of the present disclosure, when the reversible terminators are Combination 1, the use concentrations of the respective reversible terminators are as follows: the use concentration of adenine nucleotide or derivative thereof-N3-Atto647N is from 10 nmol/L to 700 nmol/L, the use concentration of thymine nucleotide or derivative thereof-N3-Biotin/SA-CY3 is from 5 nmol/L to 60 nmol/L, the use concentration of cytosine nucleotide or derivative thereof-N3-Atto532 is from 10 nmol/L to 700 nmol/L, and the use concentration of guanine nucleotide or derivative thereof-N3-SS-Atto647N is from 0.05 nmol/L to 5 nmol/L.

In scheme 2, the cleavable groups in the four types of reversible terminator are the same and are any one of the first cleavable group or the second cleavable group. In this instance, step 4 in the two-color sequencing method of the present disclosure involves performing a second reaction on the extension products.

As a possible implementation of the two-color sequencing kit of the present application, the first signal, the second signal, the third signal, the sixth signal, and the seventh signal satisfy condition 3) or condition 4), and the structures of the four types of reversible terminator satisfy the following:
the structure of the first reversible terminator is: first nucleotide or derivative thereof-first cleavable group or second cleavable group-first fluorescent group;
the structure of the second reversible terminator is: second nucleotide or derivative thereof-first cleavable group or second cleavable group-specific binding label; or second nucleotide or derivative thereof-first cleavable group or second cleavable group-first fluorescent group or second fluorescent group-specific binding label;
the structure of the third reversible terminator is: third nucleotide or derivative thereof-first cleavable group or second cleavable group-specific binding label; or third nucleotide or derivative thereof-first cleavable group or second cleavable group-second fluorescent group or first fluorescent group-specific binding label;
the structure of the fourth reversible terminator is: fourth nucleotide or derivative thereof-first cleavable group or second cleavable group-second fluorescent group;
wherein the first nucleotide or derivative thereof, the second nucleotide or derivative thereof, the third nucleotide or derivative thereof, and the fourth nucleotide or derivative thereof are selected from one of adenine or derivative thereof, thymine or derivative thereof, cytosine or derivative thereof, or guanine or derivative thereof, and the first nucleotide or derivative thereof, the second nucleotide or derivative thereof, the third nucleotide or derivative thereof, and the fourth nucleotide or derivative thereof are different from each other.

As a possible implementation of the two-color sequencing kit of the present application, the first fluorescent group and the second fluorescent group are each independently selected from: one or combinations of more of Atto532, Atto647N, Atto390, Atto Rho6G, Alexa Fluor405, Alexa Fluor532, GM656, Alexa Fluor647, CY5, IF700, ROX, CY3, CY5 rhodamine dye, cyanine dye, coumarin dye, or phycoerythrin.

As a possible implementation of the two-color sequencing kit of the present application, the fluorescent color of the first fluorescent group is red, green, or blue;
the fluorescent color of the second fluorescent group is red, green, or blue.

As a possible implementation of the two-color sequencing kit of the present application, a combination of the first fluorescent signal and the second fluorescent signal is a combination formed of red and green; or a combination of the first fluorescent signal and the second fluorescent signal is a combination formed of red and blue.

As a possible implementation of the two-color sequencing kit of the present application, the fluorescent group whose fluorescent color is green comprises Atto532, Alexa Fluor532, Atto Rho6G, or CY3.
the fluorescent group whose fluorescent color is red comprises: Atto647N, CY5, GM656, or Alexa Fluor647;
the fluorescent group whose fluorescent color is blue comprises: Atto390, Alexa Fluor405, or coumarin.

As a possible implementation of the two-color sequencing kit of the present application, the labeling substance comprises n fluorescent groups, wherein n is 1 or 2. As a possible implementation of the two-color sequencing kit of the present application, a combination of the specific binding label and the ligand is selected from at least one of streptavidin (SA)-Biotin combination, Biotin-streptavidin (SA) combination, Biotin (SA)-avidin combination, or antibody-antigen combination.

As a possible implementation of the two-color sequencing kit of the present application, a combination of specific binding label and the ligand is selected from Biotin-avidin combination.

Below, for convenience of understanding and description, the first cleavable group is exemplified by —N3- to explain Scheme 2 in conjunction with specific examples. However, it should be understood that the first cleavable group may also be replaced by the second cleavable group. The combination of the first cleavable group and the second cleavable group is not limited to the above scenarios, and other cleavable groups listed in the present disclosure may be selected. In addition, the nucleotides or derivatives thereof provided by the present disclosure may be deoxyribonucleotides or derivatives thereof, or may be ribonucleotides or derivatives thereof. For convenience of description, the following exemplifications are uniformly explained exemplified by deoxyribonucleotides or derivatives thereof. Specifically, adenine deoxyribonucleotide or derivative thereof is represented by dA, guanine deoxyribonucleotidesor derivative thereof is represented by dG, cytosine deoxyribonucleotide or derivative thereof is represented by dC, and thymine deoxyribonucleotide or derivative thereof is represented by dT.

On the basis of the foregoing, Scheme 2 of two-color sequencing of the present disclosure may involve different scenarios. In an implementation, steps and signal situations of two-color sequencing may be Scenario 2a as shown in Table 6 below:

TABLE 6
Signal changes and detection steps associated with two-color
sequencing method 2a

| Reversible | First | | Second | |
|---|---|---|---|---|
| terminators | image | Binding | image | Cleavage |
| first nucleotide or derivative | 1 first | | 1 fifth | N3 |
| thereof-N3-red fluorescent | signal | | signal | |
| group | red | | red | |
| second nucleotide or derivative | 0 second | ligand-red | 1 sixth | N3 |
| thereof-N3-specific binding | signal | fluorescent | signal | |
| label | | group | red | |
| third nucleotide or derivative | 1 third | ligand-red | 1 seventh | N3 |
| thereof-N3-green fluorescent | signal | fluorescent | signal | |
| group-specific binding label | green | group | red green | |
| fourth nucleotide or derivative | 1 fourth | | 1 eighth | N3 |
| thereof-N3-green fluorescent | signal | | signal | |
| group | green | | green | |

It should be understood that the types of fluorescent group in the reversible terminators provided in Table 6 above may be exchanged for one another, and in this instance, the combination of reversible terminators may also be as follows:
first nucleotide or derivative thereof-N3-red fluorescent group, second nucleotide or derivative thereof-N3-specific binding label, third nucleotide or derivative thereof-N3-green fluorescent group-specific binding label, and fourth nucleotide or derivative thereof-N3-green fluorescent group.

Exemplarily, the combination of reversible terminators that can realize the steps and signal situations of Scenario 2a as shown in Table 6 is selected from at least one of the following Combination 15 or Combination 16:
Combination 15: adenine nucleotide or derivative thereof-N3-Atto647N, guanine nucleotide or derivative thereof-N3-Biotin, thymine nucleotide or derivative thereof-N3-Atto532-Biotin, and cytosine nucleotide or derivative thereof-N3-Atto532;
Combination 16: adenine nucleotide or derivative thereof-N3-CY5, guanine nucleotide or derivative thereof-N3-Biotin, thymine nucleotide or derivative thereof-N3-Atto532-Biotin, and cytosine nucleotide or derivative thereof-N3-Atto532.

In an implementation, steps and signal situations of two-color sequencing may be Scenario 2b as shown in Table 7 below:

TABLE 7
Signal changes and detection steps associated with two-color sequencing method 2b

| Reversible terminators | First image | Binding | Second image | Cleavage |
|---|---|---|---|---|
| first nucleotide or | 1 first signal red | | 1 fifth signal red | N3 |
| derivative thereof-N3-red | | | | |
| fluorescent group | | | | |
| second nucleotide or | 0 second signal | ligand-red | 1 sixth signal red | N3 |
| derivative thereof-N3- | | fluorescent | | |
| specific binding label | | group | | |
| third nucleotide or | 1 third signal | ligand-green | 1 seventh signal | N3 |
| derivative thereof-N3-red | red | fluorescent | red green | |
| fluorescent group-Biotin | | group | | |
| fourth nucleotide or | 1 fourth signal | | 1 eighth signal | N3 |
| derivative thereof-N3- | green | | green | |
| green fluorescent group | | | | |

It should be understood that the types of fluorescent group in the reversible terminators provided in Table 7 above may be exchanged for one another, and in this instance, the combination of reversible terminators may also be as follows:
first nucleotide or derivative thereof-N3-red fluorescent group, second nucleotide or derivative thereof-N3-specific binding label, third nucleotide or derivative thereof-N3-red fluorescent group-Biotin, and fourth nucleotide or derivative thereof-N3-green fluorescent group.

In this instance, the labeling substance for the third reversible terminator is correspondingly: ligand-green fluorescent group, and the labeling substance for the fourth reversible terminator is correspondingly: ligand-red fluorescent group.

Exemplarily, the combination of reversible terminators that can realize the steps and signal situations of Scenario 2b as shown in Table 7 is selected from at least one of the following Combination 17 to Combination 19:
Combination 17: adenine nucleotide or derivative thereof-N3-Atto647N, guanine nucleotide or derivative thereof-N3-Biotin, thymine cytosine nucleotide or derivative thereof-N3-Atto647N-Biotin, and cytosine nucleotide or derivative thereof-N3-Atto532;
Combination 18: adenine nucleotide or derivative thereof-N3-CY5, guanine nucleotide or derivative thereof-N3-Biotin, thymine nucleotide or derivative thereof-N3-Atto647N-Biotin, and cytosine nucleotide or derivative thereof-N3-Atto532;
Combination 19: adenine nucleotide or derivative thereof-N3-Atto647N, guanine nucleotide or derivative thereof-N3-Biotin, thymine nucleotide or derivative thereof-N3-CY5-Biotin, and cytosine nucleotide or derivative thereof-N3-Atto532;
In an implementation, steps and signal situations of two-color sequencing may be Scenario 2c as shown in Table 8 below:

TABLE 8
Signal changes and detection steps associated with two-color
sequencing method 2c

| Reversible | First | | Second | |
|---|---|---|---|---|
| terminators | image | Binding | image | Cleavage |
| first | 1 first | | 1 fifth | N3 |
| nucleotide or | signal green | | signal green | |
| derivative | | | | |
| thereof-N3-red | | | | |
| fluorescent group | | | | |
| second | 0 | ligand- | 1 sixth | N3 |
| nucleotide or | second signal | red | signal red | |
| derivative | | fluorescent | | |
| thereof-N3- | | group | | |
| specific binding | | | | |
| label | | | | |
| third | 1 third | ligand- | 1 seventh | N3 |
| nucleotide or | signal green | red | signal red green | |
| derivative | | fluorescent | | |
| thereof-N3-green | | group | | |
| fluorescent | | | | |
| group-specific | | | | |
| binding label | | | | |
| fourth | 1 fourth | | 1 eighth | N3 |
| nucleotide or | signal red | | signal red | |
| derivative | | | | |
| thereof-N3-green | | | | |
| fluorescent group | | | | |

It should be understood that the types of fluorescent group in the reversible terminators provided in Table 8 above may be exchanged for one another, and in this instance, the combination of reversible terminators may also be as follows:
first nucleotide or derivative thereof-N3-red fluorescent group, second nucleotide or derivative thereof-N3-specific binding label, third nucleotide or derivative thereof-N3-green fluorescent group-specific binding label, and fourth nucleotide or derivative thereof-N3-green fluorescent group. In this instance, the labeling substance for the second reversible terminator is correspondingly: ligand-green fluorescent group, and the labeling substance for the third reversible terminator is correspondingly: ligand-green fluorescent group.

Exemplarily, the combination of reversible terminators that can realize the steps and signal situations of Scenario 2c as shown in Table 8 is selected from at least one of the following Combination 20 or Combination 21:
Combination 20: adenine nucleotide or derivative thereof-N3-Atto532, guanine nucleotide or derivative thereof-N3-Biotin, thymine cytosine nucleotide or derivative thereof-N3-Atto532-Biotin, and cytosine nucleotide or derivative thereof-N3-Atto647N;
Combination 21: adenine nucleotide or derivative thereof-N3-Atto532, guanine nucleotide or derivative thereof-N3-Biotin, thymine cytosine nucleotide or derivative thereof-N3-Atto532-Biotin, and cytosine nucleotide or derivative thereof-N3-CY5.

In an implementation, steps and signal situations of two-color sequencing may be Scenario 2d as shown in Table 9 below:

TABLE 9
Signal changes and detection steps associated with two-color sequencing method
2d

| Reversible | First | | Second | |
|---|---|---|---|---|
| terminators | image | Binding | image | Cleavage |
| first nucleotide | 1 | | 1 fifth | N3 |
| or derivative thereof- | first signal | | signal red | |
| N3-red fluorescent | red | | | |
| group | | | | |
| second | 0 | ligand-green | 1 sixth | N3 |
| nucleotide or | second | fluorescent group | signal green | |
| derivative thereof-N3- | signal | | | |
| specific binding label | | | | |
| third | 0 | ligand-green | 1 | N3 |
| nucleotide or | third signal | fluorescent group-red | seventh signal | |
| derivative thereof-N3- | | fluorescent group | red green | |
| specific binding label | | | | |
| fourth | 1 | | 1 eighth | N3 |
| nucleotide or | fourth | | signal green | |
| derivative thereof-N3- | signal green | | | |
| green fluorescent | | | | |
| group | | | | |

It should be understood that the types of fluorescent group in the reversible terminators provided in Table 9 above may be exchanged for one another, and in this instance, the combination of reversible terminators may also be as follows:
first nucleotide or derivative thereof-N3-red fluorescent group, second nucleotide or derivative thereof-N3-specific binding label, third nucleotide or derivative thereof-N3-specific binding label, and fourth nucleotide or derivative thereof-N3-green fluorescent group. In this instance, the labeling substance for the second reversible terminator is correspondingly: ligand-red fluorescent group.

Exemplarily, the combination of reversible terminators that can realize the steps and signal situations of Scenario 2d as shown in Table 9 is selected from at least one of the following Combination 22 or Combination 23:
Combination 22: adenine nucleotide or derivative thereof-N3-Atto647N, guanine nucleotide or derivative thereof-N3-Biotin, thymine cytosine nucleotide or derivative thereof-N3-Biotin, and cytosine nucleotide or derivative thereof-N3-Atto532;
Combination 23: adenine nucleotide or derivative thereof-N3-CY5, guanine nucleotide or derivative thereof-N3-Biotin, thymine nucleotide or derivative thereof-N3-Biotin, and cytosine nucleotide or derivative thereof-N3-Atto532.

In an implementation, steps and signal situations of two-color sequencing may be Case 2e as shown in Table 10 below:

TABLE 10
Signal changes and detection steps associated with two-color sequencing method 2e

| | | | Second | |
|---|---|---|---|---|
| Reversible terminators | First image | Binding | image | Cleavage |
| first nucleotide or | 1 first signal | | 1 fifth signal | N3 |
| derivative thereof-N3- | green | | green | |
| red-green fluorescent | | | | |
| group | | | | |
| second nucleotide or | 0 second | ligand-green | 1 sixth signal | N3 |
| derivative thereof-N3- | signal | fluorescent group | green | |
| specific binding label | | | | |
| third nucleotide or | 0 third signal | ligand-green | 1 seventh | N3 |
| derivative thereof-N3- | | fluorescent group-red | signal red | |
| specific binding label | | fluorescent group | green | |
| fourth nucleotide or | 1 fourth signal | | 1 eighth | N3 |
| derivative thereof-N3- | red | | signal red | |
| red fluorescent group | | | | |

It should be understood that the types of fluorescent group in the reversible terminators provided in Table 10 above may be exchanged for one another, and in this instance, the combination of reversible terminators may also be as follows:
first nucleotide or derivative thereof-N3-red fluorescent group, second nucleotide or derivative thereof-N3-specific binding label, third nucleotide or derivative thereof-N3-specific binding label, and fourth nucleotide or derivative thereof-N3-green fluorescent group. In this instance, the labeling substance for the second reversible terminator is correspondingly: ligand-red fluorescent group.

Exemplarily, the combination of reversible terminators that can realize the steps and signal situations of Scenario 2e as shown in Table 10 is selected from at least one of the following Combination 24 or Combination 25:
Combination 24: adenine nucleotide or derivative thereof-N3-Atto532, guanine nucleotide or derivative thereof-N3-Biotin, thymine cytosine nucleotide or derivative thereof-N3-Biotin, and cytosine nucleotide or derivative thereof-N3-Atto647N;
Combination 25: adenine nucleotide or derivative thereof-N3-Atto532, guanine nucleotide or derivative thereof-N3-Biotin, thymine cytosine nucleotide or derivative thereof-N3-Biotin, and cytosine nucleotide or derivative thereof-N3-CY5.

In an embodiment of Scheme 2, in Combinations 15 to 25, replacing the cleavable group —N3- with —SS— can form new combinations of reversible terminators.

Further, on the basis of the above Combinations 15 to 25, or on the basis of performing replacement of the cleavable groups on Combinations 15 to 25, performing the following operations can form new combinations of reversible terminators:
in some embodiments, in Combinations 15 to 25, CY5 and/or Atto647N are/is replaced with GM656 or Alexa Fluor647; and/or
Atto532 and/or Alexa Fluor532 are/is replaced with Atto Rho6G or CY3; or
in other embodiments, in Combinations 15 to 25, CY5 and/or Atto647N are/is replaced with one of the blue light dyes.

In Scheme 2 of the present disclosure, the reversible terminators are preferably Combination 17.

In the Combination 17, the use concentration of adenine nucleotide or derivative thereof-N3-Atto647N is from 10 nmol/L to 700 nmol/L, the use concentration of thymine or derivative thereof-N3-Atto532-Biotin is from 5 nmol/L to 60 nmol/L, the use concentration of cytosine nucleotide or derivative thereof-N3-Atto532 is from 10 nmol/L to 700 nmol/L, and the use concentration of cytosine nucleotide or derivative thereof-N3-Bitoin is from 0.05 nmol/L to 5 nmol/L.

In Scheme 1 and Scheme 2 of the present disclosure, the binding reaction comprises adding the labeling substance.

As a possible implementation of the two-color sequencing kit of the present application, the time of the binding reaction is greater than or equal to 20 s.

In Scheme 1 and Scheme 2 of the present disclosure, the cleavage reagent for cleaving the first cleavable group is an organophosphorus cleavage reagent.

As a possible implementation of the two-color sequencing kit of the present application, in the organophosphorus cleavage reagent, the organophosphorus is selected from any one or more of tris(3-hydroxypropyl)phosphine, tris(2-carboxyethyl)phosphine, trimethylolphosphine, triphenylphosphine, or tris(2-hydroxyethyl)phosphine.

As a possible implementation of the two-color sequencing kit of the present application, in the organophosphorus cleavage reagent, the organophosphorus is tris(3-hydroxypropyl)phosphine.

As a possible implementation of the two-color sequencing kit of the present application, the use concentration of the tris(3-hydroxypropyl)phosphine is from 10 mmol/L to 100 mmol/L.

In Scheme 1 and Scheme 2 of the present disclosure, the cleavage reagent for cleaving the second cleavable group is a reducing agent.

As a possible implementation of the two-color sequencing kit of the present application, in the reducing agent, the reducing agent is selected from DTT, glutathione, and/or β-SS-ethanol.

As a possible implementation of the two-color sequencing kit of the present application, the reducing agent is glutathione.

As a possible implementation of the two-color sequencing kit of the present application, the use concentration of the glutathione is from 10 mmol/L to 100 mmol/L.

The present disclosure provides a kit, the kit comprising four types of reversible terminator, a first cleavage reagent and/or a second cleavage reagent;
the four types of reversible terminator independently being: a first reversible terminator, a second reversible terminator, a third reversible terminator, and a fourth reversible terminator; the four types of reversible terminator each independently comprising: a nucleotide or derivative thereof, a labeling moiety, and a first cleavable group and/or a second cleavable group linking the nucleotide or derivative thereof and the labeling moiety, the nucleotide or derivative thereof being selected from adenine or derivative thereof, thymine or derivative thereof, cytosine or derivative thereof, or guanine or derivative thereof,
wherein,
the labeling moiety is selected from: a fluorescent group and/or a specific binding label, the specific binding label being configured to bind to a labeling substance, and the labeling substance comprising a ligand and a fluorescent group bound to the ligand;
when the reversible terminator comprises both the first cleavable group and the second cleavable group, the first cleavable group and the second cleavable group are different, and the second cleavable group has a higher cleavability than the first cleavable group;
the first cleavage reagent is a reagent for cleaving the first cleavable group or the second cleavable group; and
the second cleavage reagent is a reagent for cleaving the first cleavable group or the second cleavable group.

As a possible implementation of the two-color sequencing kit of the present application, the cleavage reagent for cleaving the first cleavable group is an organophosphorus cleavage reagent.

As a possible implementation of the two-color sequencing kit of the present application, in the organophosphorus cleavage reagent, the organophosphorus is selected from any one or more of tris(3-hydroxypropyl)phosphine, tris(2-carboxyethyl)phosphine, trimethylolphosphine, triphenylphosphine, or tris(2-hydroxyethyl)phosphine.

As a possible implementation of the two-color sequencing kit of the present application, in the organophosphorus cleavage reagent, the organophosphorus is tris(3-hydroxypropyl)phosphine.

As a possible implementation of the two-color sequencing kit of the present application, the use concentration of the tris(3-hydroxypropyl)phosphine is from 10 mmol/L to 100 mmol/L.

As a possible implementation of the two-color sequencing kit of the present application, the cleavage reagent for cleaving the second cleavable group is a reducing agent.

As a possible implementation of the two-color sequencing kit of the present application, in the reducing agent, the reducing agent is selected from DTT, glutathione, and/or β-SS-ethanol.

As a possible implementation of the two-color sequencing kit of the present application, the reducing agent is glutathione.

As a possible implementation of the two-color sequencing kit of the present application, the use concentration of the glutathione is from 10 mmol/L to 100 mmol/L.

Further, in the kit of the present disclosure, one or more of the four types of reversible terminator comprise(s) the first cleavable group, and one or more of the four types of reversible terminator comprise(s) the second cleavable group.

As a possible implementation of the two-color sequencing kit of the present application,
the structure of the first reversible terminator is: first nucleotide or derivative thereof-first cleavable group-first fluorescent group,
the structure of the second reversible terminator is one of the following scenarios: second nucleotide or derivative thereof-first cleavable group-second cleavable group-first fluorescent group or second fluorescent group; or second nucleotide or derivative thereof-first cleavable group-specific binding label; or second nucleotide or derivative thereof-first cleavable group-first fluorescent group or second fluorescent group-specific binding label;
the structure of the third reversible terminator is one of the following scenarios: third nucleotide or derivative thereof-first cleavable group-second cleavable group-first fluorescent group or second fluorescent group; or second nucleotide or derivative thereof-first cleavable group-specific binding label; or third nucleotide or derivative thereof-first cleavable group-first fluorescent group or second fluorescent group-specific binding label;
the structure of the fourth reversible terminator is: fourth nucleotide or derivative thereof-first cleavable group-second fluorescent group;
the second reversible terminator and the third reversible terminator do not comprise the specific binding label at the same time;
or
the cleavable groups in the four types of reversible terminator are the same and are any one of the first cleavable group or the second cleavable group;
the structure of the first reversible terminator is: first nucleotide or derivative thereof-first cleavable group or second cleavable group-first fluorescent group;
the structure of the second reversible terminator is: second nucleotide or derivative thereof-first cleavable group or second cleavable group-specific binding label; or second nucleotide or derivative thereof-first cleavable group or second cleavable group-first fluorescent group or second fluorescent group-specific binding label;
the structure of the third reversible terminator is: third nucleotide or derivative thereof-first cleavable group or second cleavable group-specific binding label; or third nucleotide or derivative thereof-first cleavable group or second cleavable group-second fluorescent group or first fluorescent group-specific binding label;
the structure of the fourth reversible terminator is: fourth nucleotide or derivative thereof-first cleavable group or second cleavable group-second fluorescent group;
wherein,
the first nucleotide or derivative thereof, the second nucleotide or derivative thereof, the third nucleotide or derivative thereof, and the fourth nucleotide or derivative thereof are selected from one of adenine or derivative thereof, thymine or derivative thereof, cytosine or derivative thereof, or guanine or derivative thereof, and the first nucleotide or derivative thereof, the second nucleotide or derivative thereof, the third nucleotide or derivative thereof, and the fourth nucleotide or derivative thereof are different from each other.

As a possible implementation of the two-color sequencing kit of the present application, the first cleavable group is selected from groups comprising at least one of —N₃—, —CH₂F, —C(O)NHBu, —CHF₂, —C(O)OH, —C6 and/or —ArC6, or disulfide bond.

As a possible implementation of the two-color sequencing kit of the present application, the first cleavable group is selected from at least one of —N₃—, —CH₂F, —C(O)NHBu, —CHF₂, —C(O)OH, —C₆ and/or —ArC₆, or disulfide bond.

As a possible implementation of the two-color sequencing kit of the present application, the first cleavable group is —N3- or disulfide bond.

As a possible implementation of the two-color sequencing kit of the present application, the second cleavable group is selected from groups comprising disulfide bond.

As a possible implementation of the two-color sequencing kit of the present application, the second cleavable group is disulfide bond (—SS—).

As a possible implementation of the two-color sequencing kit of the present application, the fluorescent group is selected from a first fluorescent group and a second fluorescent group, which are distinguishable in color.

As a possible implementation of the two-color sequencing kit of the present application, the first fluorescent group and the second fluorescent group are each independently selected from: one or combinations of more of Atto532, Atto647N, Atto390, Atto Rho6G, Alexa Fluor405, Alexa Fluor532, GM656, Alexa Fluor647, CY5, IF700, ROX, CY3, CY5 rhodamine dye, cyanine dye, coumarin dye, or phycoerythrin.

As a possible implementation of the two-color sequencing kit of the present application, the fluorescent color of the first fluorescent group is red, green, or blue;
the fluorescent color of the second fluorescent group is red, green, or blue.

As a possible implementation of the two-color sequencing kit of the present application, a combination of the first fluorescent signal and the second fluorescent signal is a combination formed of red and green; or a combination of the first fluorescent signal and the second fluorescent signal is a combination formed of red and blue.

As a possible implementation of the two-color sequencing kit of the present application, the fluorescent group whose fluorescent color is green comprises Atto532, Alexa Fluor532, Atto Rho6G, or CY3;
the fluorescent group whose fluorescent color is red comprises: Atto647N, CY5, GM656, or Alexa Fluor647;
the fluorescent group whose fluorescent color is blue comprises: Atto390, Alexa Fluor405, or coumarin.

As a possible implementation of the two-color sequencing kit of the present application, the labeling substance comprises n fluorescent groups, wherein n is 1 or 2.

As a possible implementation of the two-color sequencing kit of the present application, a combination of specific binding label and the ligand is selected from at least one of Biotin-streptavidin (SA) combination, Biotin (SA)-streptavidin (Biotin) combination, Biotin-avidin combination, or antibody-antigen combination.

As a possible implementation of the two-color sequencing kit of the present application, a combination of specific binding label and the ligand is selected from Biotin-streptavidin (SA) combination.

Further, in the kit of the present disclosure,
the reversible terminators comprise at least one of the following Combinations 1 to 15, or at least one of the following Combinations 15 to 25:
Combination 1: adenine nucleotide or derivative thereof-N3-Atto647N, guanine nucleotide or derivative thereof-N3-SS-Atto647N, thymine nucleotide or derivative thereof-N3-Biotin, and cytosine nucleotide or derivative thereof-N3-Atto532;
Combination 2: adenine nucleotide or derivative thereof-N3-CY5, guanine nucleotide or derivative thereof-N3-SS-Atto647N, thymine nucleotide or derivative thereof-N3-Biotin, and cytosine nucleotide or derivative thereof-N3-Atto532;
Combination 3: adenine nucleotide or derivative thereof-N3-CY5, guanine nucleotide or derivative thereof-N3-SS-CY5, thymine nucleotide or derivative thereof-N3-Biotin, and cytosine nucleotide or derivative thereof-N3-Atto532;
Combination 4: adenine nucleotide or derivative thereof-N3-Atto647N, guanine nucleotide or derivative thereof-N3-SS-CY5, thymine nucleotide or derivative thereof-N3-Biotin, and cytosine nucleotide or derivative thereof-N3-Atto532;
Combination 5: adenine nucleotide or derivative thereof-N3-Atto647N, guanine nucleotide or derivative thereof-N3-SS-Atto647N, thymine nucleotide or derivative thereof-N3-Atto532-Biotin, and cytosine nucleotide or derivative thereof-N3-Atto532;
Combination 6: adenine nucleotide or derivative thereof-N3-Atto647N, guanine nucleotide or derivative thereof-N3-SS-CY5, cytosine nucleotide or derivative thereof-N3-Atto532-Biotin, and cytosine nucleotide or derivative thereof-N3-Atto532;
Combination 7: adenine nucleotide or derivative thereof-N3-CY5, guanine nucleotide or derivative thereof-N3-SS-Atto647N, thymine nucleotide or derivative thereof-N3-Atto532-Biotin, and cytosine nucleotide or derivative thereof-N3-Atto532;
Combination 8: adenine nucleotide or derivative thereof-N3-CY5, guanine nucleotide or derivative thereof-N3-SS-CY5, thymine nucleotide or derivative thereof-N3-Atto532-Biotin, and cytosine nucleotide or derivative thereof-N3-Atto532;
Combination 9: adenine nucleotide or derivative thereof-N3-Atto647N, guanine nucleotide or derivative thereof-N3-SS-Atto532, thymine nucleotide or derivative thereof-N3-Biotin, and cytosine nucleotide or derivative thereof-N3-Atto532;
Combination 10: adenine nucleotide or derivative thereof-N3-CY5, guanine nucleotide or derivative thereof-N3-SS-Atto532, thymine nucleotide or derivative thereof-N3-Biotin, and cytosine nucleotide or derivative thereof-N3-Atto532;
Combination 11: adenine nucleotide or derivative thereof-N3-Atto532, guanine nucleotide or derivative thereof-N3-SS-Atto532, cytosine nucleotide or derivative thereof-N3-Biotin, and cytosine nucleotide or derivative thereof-N3-Atto647N;
Combination 12: adenine nucleotide or derivative thereof-N3-Atto532, guanine nucleotide or derivative thereof-N3-SS-Atto532, thymine nucleotide or derivative thereof-N3-Biotin, and cytosine nucleotide or derivative thereof-N3-CY5;
Combination 13: adenine nucleotide or derivative thereof-N3-Atto532, guanine nucleotide or derivative thereof-N3-SS-Atto532, cytosine nucleotide or derivative thereof-N3-Atto647N-Biotin, and cytosine nucleotide or derivative thereof-N3-Atto647N;
Combination 14: adenine nucleotide or derivative thereof-N3-Atto532, guanine nucleotide or derivative thereof-N3-Atto532, thymine cytosine nucleotide or derivative thereof-N3-CY5-Biotin, and cytosine nucleotide or derivative thereof-N3-CY5.
Combination 15: adenine nucleotide or derivative thereof-N3-Atto647N, guanine nucleotide or derivative thereof-N3-Biotin, thymine nucleotide or derivative thereof-N3-Atto532-Biotin, and cytosine nucleotide or derivative thereof-N3-Atto532;
Combination 16: adenine nucleotide or derivative thereof-N3-CY5, guanine nucleotide or derivative thereof-N3-Biotin, thymine nucleotide or derivative thereof-N3-Atto532-Biotin, and cytosine nucleotide or derivative thereof-N3-Atto532;
Combination 17: adenine nucleotide or derivative thereof-N3-Atto647N, guanine nucleotide or derivative thereof-N3-Biotin, thymine cytosine nucleotide or derivative thereof-N3-Atto647N-Biotin, and cytosine nucleotide or derivative thereof-N3-Atto532;
Combination 18: adenine nucleotide or derivative thereof-N3-CY5, guanine nucleotide or derivative thereof-N3-Biotin, thymine nucleotide or derivative thereof-N3-Atto647N-Biotin, and cytosine nucleotide or derivative thereof-N3-Atto532;
Combination 19: adenine nucleotide or derivative thereof-N3-Atto647N, guanine nucleotide or derivative thereof-N3-Biotin, thymine nucleotide or derivative thereof-N3-CY5-Biotin, and cytosine nucleotide or derivative thereof-N3-Atto532;
Combination 20: adenine nucleotide or derivative thereof-N3-Atto532, guanine nucleotide or derivative thereof-N3-Biotin, thymine cytosine nucleotide or derivative thereof-N3-Atto532-Biotin, and cytosine nucleotide or derivative thereof-N3-Atto647N;
Combination 21: Adenine nucleotide or derivative thereof-N3-Atto532, guanine nucleotide or derivative thereof-N3-Biotin, thymine cytosine nucleotide or derivative thereof-N3-Atto532-Biotin, and cytosine nucleotide or derivative thereof-N3-CY5.
Combination 22: adenine nucleotide or derivative thereof-N3-Atto647N, guanine nucleotide or derivative thereof-N3-Biotin, thymine cytosine nucleotide or derivative thereof-N3-Biotin, and cytosine nucleotide or derivative thereof-N3-Atto532;
Combination 23: adenine nucleotide or derivative thereof-N3-Atto647N, guanine nucleotide or derivative thereof-N3-Biotin, thymine cytosine nucleotide or derivative thereof-N3-Biotin, and cytosine nucleotide or derivative thereof-N3-Atto532;
Combination 24: adenine nucleotide or derivative thereof-N3-Atto532, guanine nucleotide or derivative thereof-N3-Biotin, thymine cytosine nucleotide or derivative thereof-N3-Biotin, and cytosine nucleotide or derivative thereof-N3-Atto647N;
Combination 25: adenine nucleotide or derivative thereof-N3-Atto532, guanine nucleotide or derivative thereof-N3-Biotin, thymine cytosine nucleotide or derivative thereof-N3-Biotin, and cytosine nucleotide or derivative thereof-N3-CY5.

As a possible implementation of the two-color sequencing kit of the present application, in Combinations for the kit of the present disclosure, performing replacement of the cleavable groups as follows can form new combinations:
in Combinations 1 to 14, the cleavable group —N3- in any one of the reversible terminators not having the specific binding label is replaced with —N3-SS—, and the cleavable group —N3-SS— in any one of the reversible terminators not having the specific binding label is replaced with —N3-; and/or
in Combinations 1 to 14, —N3- linked to cytosine or derivative thereof or pyrimidine or derivative thereof is replaced with —N3-SS—, and —N3-SS— linked to guanine nucleotide or derivative thereof is replaced with —N3-;
or, in Combinations 15 to 25, the cleavable group —N3- is replaced with —SS—.

Still further, on the basis of Combinations 1 to 25, or after performing replacement of the cleavable groups on Combinations 1 to 25, performing replacement as follows can form new combinations:
in Combinations 1 to 25, CY5 and/or Atto647N are/is replaced with GM656 or Alexa Fluor647; and/or
Atto532 and/or Alexa Fluor532 are/is replaced with Atto Rho6G or CY3; or
in Combinations 1 to 25, CY5 and/or Atto647N are/is replaced with one of the blue light dyes.

In the kit of the present disclosure, the combination is preferably Combination 1, and in the Combination 1, the use concentration of adenine nucleotide or derivative thereof-N3-Atto647N is from 10 nmol/L to 700 nmol/L, the use concentration of thymine-N3-Biotin/SA-CY3 is from 5 nmol/L to 60 nmol/L, the use concentration of cytosine nucleotide or derivative thereof-N3-Atto532 is from 10 nmol/L to 700 nmol/L, and the use concentration of guanine nucleotide or derivative thereof-N3-SS-Atto647N is from 0.05 nmol/L to 5 nmol/L.

In the present disclosure, the types of reversible terminator in the two-color sequencing method are associated with the specific steps of the two-color sequencing method. Sequencing may not be achieved if the types of reversible terminator are changed. In the present disclosure, through optimization of the concentrations of the reversible terminators, optimization of the concentrations of the first cleavage reagent and the second cleavage reagent, optimization of exposure time and wavelength, etc., a two-color sequencing method for single-molecule sequencing, which is adapted to the reversible terminator system of the present disclosure, is obtained.

In the present disclosure, through reasonably designing the reversible terminators participating in the reactions in the two-color sequencing method and optimizing the reagents and reaction conditions for the two-color sequencing method, the two-color sequencing method is obtained suitable for a single-molecule sequencing platform. Using the two-color sequencing method in single-molecule sequencing can help to reduce the error ratio, improve the sequencing quality, and shorten the sequencing time.

### BRIEF DESCRIPTION OF THE DRAWINGS

FIG. 1 shows imaged pictures of two-color sequencing adsorption tests performed by using dA-N3-Atto647N at a concentration of 500 nM and dA-N3-Atto647N at a concentration of 10 nM, respectively, provided in Example 1 of the present application, wherein the left panel corresponds to 500 nM dA-N3-Atto647N, and the right panel corresponds to 10 nM dA-N3-Atto647N;

FIG. 2 shows a statistical diagram of data of cleavage effect in a test of the efficiency of cleavage of disulfide bond by glutathione (GSH), provided in Example 2 of the present application;

FIG. 3 shows a statistical diagram of data of cleavage effect in a test of the efficiency of cleavage of azidomethoxy group by THPP, provided in Example 3 of the present application;

FIG. 4 shows a statistical diagram of data of binding effect in terms of the efficiency of binding of SA-CY3 with dUTP-Biotin, provided in Example 4 of the present application; and

FIG. 5 shows a statistical diagram of binding efficiency of SA-CY3 with dUTP-Biotin at different binding times, provided in Example 5 of the present application.

### DETAILED DESCRIPTION OF THE INVENTION

Examples of the present application disclose a single-molecule two-color sequencing method based on reversible terminator nucleotides and derivatives thereof.

The terms used in the examples of the present application are merely for the purpose of describing the specific examples, and are not intended to limit the present application. The term “and/of” describes an association relationship of associated subjects, indicating that there may be three relationships. For example, A and/or B may represent: a situation where A is present alone, a situation where A and B are present together, and a situation where B is present alone. In addition, A and B may be singular or plural. The character “/” in “and/of” indicates that the associated subjects before and after same are in an “and” relationship.

The terms “first” and “second” are used for descriptive purpose only to distinguish objects such as substances and subjects from one another, and should not be understood as indicating or implying relative importance or implicitly specifying the number of the indicated technical features. For example, without departing from the scope of the examples of the present application, the first reversible terminator may also be referred to as the second reversible terminator, and the second reversible terminator may be referred to as the first reversible terminator. Thus, features defined by “first” and “second” may explicitly or implicitly comprise one or more of the features.

The term “at least one (type)” refers to one or a plurality of (types), and “a plurality of (types)” refers to two (types) or more than two (types). “At least one (type)” or a similar expression thereof refers to any combination of these items, comprising a single item (type) or any combination of a plurality of items (types). For example, “at least one (type) of a, b, or c” or “at least one (type) of a, b, and c” may both denote: a, b, c, a-b (i.e., a and b), a-c, b-c, or a-b-c, wherein a, b, and c may each be a single item (type) or a plurality of items (types).

The singular forms of “a”, “said” and “the” used in examples of the present application and the appended claims are also intended to comprise plural forms, unless the context clearly indicates otherwise.

It should be understood that, in various examples of the present application, the sequence numbers of the above respective processes do not mean the order of execution, and some or all of the steps may be executed in parallel or sequentially. The order of execution of the respective processes should be determined by the function and internal logic thereof, and should not constitute any limitation on the execution process of the examples of the present application.

The term “sequencing” used in embodiments of the present application may also be referred to as “nucleic acid sequencing” or “gene sequencing”. That is, the three terms are semantically interchangeable, and refer to determination of the type and arrangement order of the bases in a nucleic acid sequence, comprising a process of allowing nucleotides (including nucleotide analogs) to bind to a template and collecting corresponding signals emitted by the nucleotides (including analogs). Modes of “sequencing” comprise sequencing by synthesis (SBS) and/or sequencing by ligation (SBL), comprise DNA sequencing and/or RNA sequencing, and comprise long fragment sequencing and/or short fragment sequencing. The so-called long fragment and short fragment are in a relative sense. For example, a nucleic acid molecule longer than 1 Kb, 2 Kb, 5 Kb or 10 Kb may be referred to as a long fragment, and a nucleic acid molecule shorter than 1 Kb or 800 bp may be referred to as a short fragment. Modes of “sequencing” also comprise double-end sequencing, single-end sequencing and/or paired-end sequencing, etc. The so-called double-end sequencing or paired-end sequencing may refer to readout of any two segments or portions of the same nucleic acid molecule which do not completely overlap.

Sequencing generally comprises multiple cycles of a process, to achieve determination of the types and arrangement order of multiple bases or nucleotides on a nucleic acid template. In examples of the present application, each cycle of the “process to achieve determination of the types and arrangement order of multiple bases or nucleotides on a nucleic acid template” is referred to as “one cycle of sequencing”. A “cycle”, also referred to as a “sequencing cycle”, can be defined as one instance of base extension of the four kinds of nucleotide/base. In other words, a “sequencing cycle” can be defined as completing the determination of the type of the base or nucleotide at any designated position on the template. For a sequencing platform that achieves sequencing based on a polymerization or ligation reaction, one cycle of sequencing comprises a process of achieving binding of the four kinds of nucleotide (including nucleotide analogs) to the so-called nucleic acid template in a base-complementary manner at one time, and collecting the corresponding signals that are emitted. For a platform that achieves sequencing based on a polymerase chain reaction, the reaction system comprises nucleotides as reaction substrates, a polymerase, and a nucleic acid template onto which a segment of sequence (sequencing primer) is bound. On the basis of the principle of base pairing and the mechanism of the polymerase chain reaction, an added nucleotide as a reaction substrate, catalyzed by the polymerase, is linked to the sequencing primer to achieve binding of the nucleotide to a particular position of the nucleic acid template. Generally, one cycle of sequencing may comprise one instance or multiple instances of base extension (repeat). For example, the four kinds of nucleotide are sequentially added to a reaction system, and base extension and collection of a corresponding reaction signal are performed for each; one cycle of sequencing comprises four instances of base extension. Alternatively, for example, any combination of the four kinds of nucleotide is added to a reaction system. For example, the four kinds of nucleotide are added in sets of two and two, or sets of one and three, and base extension and collection of corresponding reaction signals are performed for the two sets respectively; one cycle of sequencing comprises two instances of base extension. As another example, the four kinds of nucleotide are added to a reaction system at the same time for base extension and collection of reaction signals; one cycle of sequencing comprises one instance of base extension.

The term “nucleotide” means a molecule comprising a sugar and at least one phosphate ester group, and further comprising a nucleobase. Nucleotides include deoxyribonucleotides, modified deoxyribonucleotides, ribonucleotides, modified ribonucleotides, peptide nucleotides, modified peptide nucleotides, modified sugar-phosphate backbone nucleotides, and mixtures thereof. Examples of nucleotides include adenosine monophosphate (AMP), adenosine diphosphate (ADP), adenosine triphosphate (ATP), thymidine monophosphate (TMP), thymidine diphosphate (TDP), thymidine triphosphate (TTP), cytidine monophosphate (CMP), cytidine diphosphate (CDP), cytidine triphosphate (CTP), guanosine monophosphate (GMP), guanosine diphosphate (GDP), guanosine triphosphate (GTP), uridine monophosphate (UMP), uridine diphosphate (UDP), uridine triphosphate (UTP), deoxyadenosine monophosphate (dAMP), deoxyadenosine diphosphate (dADP), deoxyadenosine triphosphate (dATP), deoxythymidine monophosphate (dTMP), deoxythymidine diphosphate (dTDP), deoxythymidine triphosphate (dTTP), deoxycytidine diphosphate (dCDP), deoxycytidine triphosphate (dCTP), deoxyguanosine monophosphate (dGMP), deoxyguanosine diphosphate (dGDP), deoxyguanosine triphosphate (dGTP), deoxyuridine monophosphate (dUMP), deoxyuridine diphosphate (dUDP), and deoxyuridine triphosphate (dUTP).

As used herein, the term “nucleotide” is also intended to encompass any nucleotide analog, which is the type of nucleotide that comprises a nucleobase, sugar, and/or phosphate ester moiety that is modified as compared to a naturally occurring nucleotide. Exemplary modified nucleobases include inosine, xanthine, hypoxanthine, isocytosine, isoguanine, 2-aminopurine, 5-methylcytosine, 5-hydroxymethylcytosine, 2-aminoadenine, 6-methyladenine, 6-methylguanine, 2-propylguanine, 2-propyladenine, 2-thiouracil, 2-thiothymine, 2-thiocytosine, 15-halouracil, 15-halocytosine, 5-propynyluracil, 5-propynylcytosine, 6-azouracil, 6-azocytosine, 6-azothymine, 5-uracil, 4-thiouracil, 8-haloadenine or guanine, 8-aminoadenine or guanine, 8-thioladenine or guanine, 8-sulfanyladenine or guanine, 8-hydroxyadenine or guanine, 5-halo-substituted uracil or cytosine, 7-methylguanine, 7-methyladenine, 8-azaguanine, 8-azadenine, 7-deazaguanine, 7-deazadenine, 3-deazaguanine, 3-deazadenine, and the like. As is known in the art, some nucleotide analogs cannot be incorporated into a polynucleotide, such as the nucleotide analog 5′-adenosine phosphosulfate. A nucleotide may comprise any suitable number of phosphate esters, for example three, four, five, six, or more than six phosphate esters.

The term “cluster” refers to a population of nucleic acid molecules attached to a surface of a solid-phase substrate to form a features or site. The nucleic acid molecules in the population of nucleic acid molecules are typically nucleic acid molecules having the same sequence, forming a monoclonal cluster by amplification or cloning. A cluster which is not a monoclonal cluster typically mainly comprises amplicons from a first nucleic acid, and may also have a low level of contaminating amplicons from a second nucleic acid. For example, when a cluster array is used in a detection application, an acceptable level of contamination would be a level that does not affect the signal-to-noise ratio or resolution of the detection technique in an unacceptable manner. Thus, apparent clonality is generally associated with a particular use or application of an array prepared by the method set forth herein.

The term “primer”, also known as “probe”, refers to: an oligonucleotide or nucleic acid molecule that can be hybridized with a target sequence of interest. In examples, a primer functions as a substrate onto which nucleotides can be polymerized by a polymerase. For example, the primer may serve as a starting point for DNA or RNA synthesis. For example, a sequencing primer can hybridize to a synthetic nucleic acid template strand so as to prime the synthesis of a new strand that is complementary to the synthetic nucleic acid template strand. The primer may comprise any combination of nucleotides or analogs thereof. In some exemplifications, the primer is a single-stranded oligonucleotide or polynucleotide.

The term “comprising”, “having”, or “containing”, including the use of grammatical synonyms thereof, should generally be understood to be open-ended and non-limiting, e.g., not excluding other unrecited elements or steps, unless specifically stated otherwise or otherwise understood from the context. The use of any and all exemplifications or exemplary language herein, such as “for example” or “including”, is intended merely to better explain the present application and does not constitute a limitation on the scope of the present application, unless otherwise claimed. No language in the specification should be construed as indicating any non-claimed element as essential to the practice of the present application.

In addition, numerical ranges and parameters used to define the present application are all approximate values, and the relevant values in the specific examples have been presented as accurately as possible herein. However, any numerical value inherently unavoidably contains standard deviations resulting from individual testing methods. Therefore, unless otherwise expressly stated, it should be understood that all ranges, amounts, values, and percentages used in the present disclosure are modified by “about”. As used herein, “about” generally means that the actual value is within plus or minus 10%, 5%, 1%, or 0.5% of a particular value or range.

The two-color sequencing method will be described below by taking sequencing by synthesis as an example.

A two-color sequencing method is provided, comprising the following steps:
(a) allowing four types of reversible terminator to undergo polymerization reactions with nucleic acid molecules attached to a surface, the four types of reversible terminator each independently comprising: a nucleotide or derivative thereof, a labeling moiety, and a first cleavable group linking the nucleotide or derivative thereof and the labeling moiety, wherein the labeling moiety is selected from a fluorescent group and/or a specific binding label, at least two of the four types of modified nucleotide carry a first fluorescent group and a second fluorescent group, which are optically distinguishable, respectively, one or two of the four types of modified nucleotide carry the specific binding label, and the specific binding label is capable of specifically binding to a corresponding ligand;
(b) exciting the fluorescent group to luminesce, and collecting a luminescence signal to obtain a first image;
(c) adding a labeling substance, the labeling substance comprising the ligand and a fluorescent group bound to the ligand, and allowing the ligand of the labeling substance to bind to the specific binding label, wherein the fluorescent group bound to the ligand is selected from the first fluorescent group or the second fluorescent group;
(d) exciting the fluorescent group to luminesce, and collecting a luminescence signal from the fluorescent group to obtain a second image; and
(e) determining the types of reversible terminator participating in the polymerization reactions on the basis of the luminescence signals and a change thereof at the same position in the first image and the second image.

As a possible implementation of the two-color sequencing method of the present application, the labeling moieties of two of the four types of modified nucleotide are the first fluorescent group and the second fluorescent group, which are optically distinguishable, respectively, and the labeling moieties of the other two of the four types of modified nucleotide are specific binding labels with different modes of specific binding, and
in step (c), the labeling substance includes two types, and the two types of labeling substance carry the first fluorescent group and the second fluorescent group, respectively; or
two of the four types of modified nucleotide each independently carry the first fluorescent group, one of the four types of modified nucleotide carries the second fluorescent group, and the other of the four types of modified nucleotide carries the specific binding label, and
in step (c), the labeling substance includes one type, and the labeling substance carries the second fluorescent group.

As a possible implementation of the two-color sequencing method of the present application, three of the four types of modified nucleotide carry the first fluorescent group and the second fluorescent group, which are optically distinguishable, respectively, and the other of the four types of modified nucleotide carries the specific binding label, and one of the two types of modified nucleotide carrying the same fluorescent group comprises a second cleavable group, the second cleavable group being so located as to link the first cleavable group and the labeling moiety, and the second cleavable group having a higher cleavability than the first cleavable group, and
step (c) further comprises: performing a cleavage reaction on the second cleavable group.

In an embodiment, the determining the types of reversible terminator participating in the polymerization reactions on the basis of the luminescence signals and a change thereof at the same position in the first image and the second image comprises:
with respect to a first position where the optical signals remain unchanged in both the first image and the second image and are both indicative of the optical signal of the first fluorescent group, determining the reversible terminator participating in the polymerization reaction at that position to be a first reversible terminator;
with respect to a second position where the optical signals remain unchanged in both the first image and the second image and are both indicative of the optical signal of the second fluorescent group, determining the reversible terminator participating in the polymerization reaction at that position to be a second reversible terminator; and
with respect to a third position and a fourth position where the optical signals become changed but follow different patterns of change between the first image and the second image, determining the reversible terminators participating in the polymerization reactions at that two positions to be a third reversible terminator and a fourth reversible terminator, respectively.

As a possible implementation of the two-color sequencing method of the present application, the structures of the four types of reversible terminator satisfy one or more of the following combinations (1)-(4):
(1) the structure of the first reversible terminator is: first nucleotide or derivative thereof-first cleavable group-first fluorescent group,
the structure of the second reversible terminator is: second nucleotide or derivative thereof-first cleavable group-specific binding label,
the structure of the third reversible terminator is: third nucleotide or derivative thereof-first cleavable group-specific binding label, and
the structure of the second reversible terminator is: fourth nucleotide or derivative thereof-first cleavable group-second fluorescent group;
(2) the structure of the first reversible terminator is: first nucleotide or derivative thereof-first cleavable group-first fluorescent group,
the structure of the second reversible terminator is: second nucleotide or derivative thereof-first cleavable group-specific binding label,
the structure of the third reversible terminator is: third nucleotide or derivative thereof-first cleavable group-specific binding label, and
the structure of the second reversible terminator is: fourth nucleotide or derivative thereof-first cleavable group-second fluorescent group-specific binding label;
(3) the structure of the first reversible terminator is: first nucleotide or derivative thereof-first cleavable group-first fluorescent group,
the structure of the second reversible terminator is: second nucleotide or derivative thereof-first cleavable group-specific binding label,
the structure of the third reversible terminator is: third nucleotide or derivative thereof-first cleavable group-second fluorescent group, and
the structure of the second reversible terminator is: fourth nucleotide or derivative thereof-first cleavable group-second cleavable group-first fluorescent group-specific binding label;
(4) the structure of the first reversible terminator is: first nucleotide or derivative thereof-first cleavable group-first fluorescent group,
the structure of the second reversible terminator is: second nucleotide or derivative thereof-first cleavable group-second fluorescent group-specific binding label,
the structure of the third reversible terminator is: third nucleotide or derivative thereof-first cleavable group-second fluorescent group, and
the structure of the second reversible terminator is: fourth nucleotide or derivative thereof-first cleavable group-second cleavable group-first fluorescent group;
Wherein in each combination, the first nucleotide or derivative thereof, the second nucleotide or derivative thereof, the third nucleotide or derivative thereof, and the fourth nucleotide or derivative thereof are selected from one of adenine or derivative thereof, thymine or derivative thereof, cytosine or derivative thereof, or guanine or derivative thereof, and the first nucleotide or derivative thereof, the second nucleotide or derivative thereof, the third nucleotide or derivative thereof, and the fourth nucleotide or derivative thereof are different from each other.

On the basis of any one of the above embodiments, as a possible implementation of the two-color sequencing method of the present application, the first cleavable group is selected from groups comprising at least one of —N₃—, —CH₂F, —C(O)NHBu, —CHF₂, —C(O)OH, —C₆—, —ArC₆—, and/or disulfide bond.

As a possible implementation of the two-color sequencing kit of the present application, first cleavable group is selected from at least one of —N₃—, —CH₂F, —C(O)NHBu, —CHF₂, —C(O)OH, —C₆—, —ArC₆, and/or disulfide.

As a possible implementation of the two-color sequencing kit of the present application, the four types of reversible terminator all comprise only the first cleavable group, and the first cleavable group is —N₃— or disulfide bond.

As a possible implementation of the two-color sequencing kit of the present application, the four types of reversible terminator each independently comprise the first cleavable group and/or the second cleavable group, the first cleavable group is —N₃—, and the second cleavable group is selected from groups comprising disulfide bond. As a possible implementation of the two-color sequencing kit of the present application, the second cleavable group is disulfide bond.

On the basis of any one of the above embodiments, as a possible implementation of the two-color sequencing method of the present application, the first fluorescent group and the second fluorescent group are each independently selected from one of a red fluorescent group, a green fluorescent group, and a blue fluorescent group.

As a possible implementation of the two-color sequencing kit of the present application, the first fluorescent group and second fluorescent group are each independently selected from: one or combinations of more of Atto532, Atto647N, Atto390, Atto Rho6G, Alexa Fluor405, Alexa Fluor532, GM656, Alexa Fluor647, CY5, IF700, ROX, CY3, CY5 rhodamine dye, cyanine dye, coumarin dye, or phycoerythrin.

As a possible implementation of the two-color sequencing kit of the present application, a combination of the first fluorescent signal and the second fluorescent signal is a combination formed of red and green; or a combination of the first fluorescent signal and the second fluorescent signal is a combination formed of red and blue.

As a possible implementation of the two-color sequencing kit of the present application, the fluorescent group whose fluorescent color is green includes Atto532, Alexa Fluor532, Atto Rho6G, or CY3;
the fluorescent group whose fluorescent color is red includes: Atto647N, CY5, GM656, or Alexa Fluor647;
the fluorescent group whose fluorescent color is blue includes: Atto390, Alexa Fluor405, or coumarin.

On the basis of any one of the above embodiments, as a possible implementation of the two-color sequencing method of the present application, the specific binding label is selected from at least one of biotin, digoxin, hapten, or functional derivatives thereof.

On the basis of any one of the above embodiments, as a possible implementation of the two-color sequencing method of the present application, the reversible terminators include at least one group shown in the following Combinations 1 to 25:
Combination 1: adenine nucleotide or derivative thereof-N3-Atto647N, guanine nucleotide or derivative thereof-N3-SS-Atto647N, thymine nucleotide or derivative thereof-N3-Biotin, and cytosine nucleotide or derivative thereof-N3-Atto532;
Combination 2: adenine nucleotide or derivative thereof-N3-CY5, guanine nucleotide or derivative thereof-N3-SS-Atto647N, thymine nucleotide or derivative thereof-N3-Biotin, and cytosine nucleotide or derivative thereof-N3-Atto532;
Combination 3: adenine nucleotide or derivative thereof-N3-CY5, guanine nucleotide or derivative thereof-N3-SS-CY5, thymine nucleotide or derivative thereof-N3-Biotin, and cytosine nucleotide or derivative thereof-N3-Atto532;
Combination 4: adenine nucleotide or derivative thereof-N3-Atto647N, guanine nucleotide or derivative thereof-N3-SS-CY5, thymine nucleotide or derivative thereof-N3-Biotin, and cytosine nucleotide or derivative thereof-N3-Atto532;
Combination 5: adenine nucleotide or derivative thereof-N3-Atto647N, guanine nucleotide or derivative thereof-N3-SS-Atto647N, thymine nucleotide or derivative thereof-N3-Atto532-Biotin, and cytosine nucleotide or derivative thereof-N3-Atto532;
Combination 6: adenine nucleotide or derivative thereof-N3-Atto647N, guanine nucleotide or derivative thereof-N3-SS-CY5, cytosine nucleotide or derivative thereof-N3-Atto532-Biotin, and cytosine nucleotide or derivative thereof-N3-Atto532;
Combination 7: adenine nucleotide or derivative thereof-N3-CY5, guanine nucleotide or derivative thereof-N3-SS-Atto647N, thymine nucleotide or derivative thereof-N3-Atto532-Biotin, and cytosine nucleotide or derivative thereof-N3-Atto532;
Combination 8: adenine nucleotide or derivative thereof-N3-CY5, guanine nucleotide or derivative thereof-N3-SS-CY5, thymine nucleotide or derivative thereof-N3-Atto532-Biotin, and cytosine nucleotide or derivative thereof-N3-Atto532;
Combination 9: adenine nucleotide or derivative thereof-N3-Atto647N, guanine nucleotide or derivative thereof-N3-SS-Atto532, thymine nucleotide or derivative thereof-N3-Biotin, and cytosine nucleotide or derivative thereof-N3-Atto532;
Combination 10: adenine nucleotide or derivative thereof-N3-CY5, guanine nucleotide or derivative thereof-N3-SS-Atto532, thymine nucleotide or derivative thereof-N3-Biotin, and cytosine nucleotide or derivative thereof-N3-Atto532;
Combination 11: adenine nucleotide or derivative thereof-N3-Atto532, guanine nucleotide or derivative thereof-N3-SS-Atto532, cytosine nucleotide or derivative thereof-N3-Biotin, and cytosine nucleotide or derivative thereof-N3-Atto647N;
Combination 12: adenine nucleotide or derivative thereof-N3-Atto532, guanine nucleotide or derivative thereof-N3-SS-Atto532, thymine nucleotide or derivative thereof-N3-Biotin, and cytosine nucleotide or derivative thereof-N3-CY5;
Combination 13: adenine nucleotide or derivative thereof-N3-Atto532, guanine nucleotide or derivative thereof-N3-SS-Atto532, cytosine nucleotide or derivative thereof-N3-Atto647N-Biotin, and cytosine nucleotide or derivative thereof-N3-Atto647N;
Combination 14: adenine nucleotide or derivative thereof-N3-Atto532, guanine nucleotide or derivative thereof-N3-SS-Atto532, thymine cytosine nucleotide or derivative thereof-N3-CY5-Biotin, and cytosine nucleotide or derivative thereof-N3-CY5;
Combination 15: adenine nucleotide or derivative thereof-N3-Atto647N, guanine nucleotide or derivative thereof-N3-Biotin, thymine nucleotide or derivative thereof-N3-Atto532-Biotin, and cytosine nucleotide or derivative thereof-N3-Atto532;
Combination 16: adenine nucleotide or derivative thereof-N3-CY5, guanine nucleotide or derivative thereof-N3-Biotin, thymine nucleotide or derivative thereof-N3-Atto532-Biotin, and cytosine nucleotide or derivative thereof-N3-Atto532;
Combination 17: adenine nucleotide or derivative thereof-N3-Atto647N, guanine nucleotide or derivative thereof-N3-Biotin, thymine cytosine nucleotide or derivative thereof-N3-Atto647N-Biotin, and cytosine nucleotide or derivative thereof-N3-Atto532;
Combination 18: adenine nucleotide or derivative thereof-N3-CY5, guanine nucleotide or derivative thereof-N3-Biotin, thymine nucleotide or derivative thereof-N3-Atto647N-Biotin, and cytosine nucleotide or derivative thereof-N3-Atto532;
Combination 19: adenine nucleotide or derivative thereof-N3-Atto647N, guanine nucleotide or derivative thereof-N3-Biotin, thymine nucleotide or derivative thereof-N3-CY5-Biotin, and cytosine nucleotide or derivative thereof-N3-Atto532;
Combination 20: adenine nucleotide or derivative thereof-N3-Atto532, guanine nucleotide or derivative thereof-N3-Biotin, thymine cytosine nucleotide or derivative thereof-N3-Atto532-Biotin, and cytosine nucleotide or derivative thereof-N3-Atto647N;
Combination 21: adenine nucleotide or derivative thereof-N3-Atto532, guanine nucleotide or derivative thereof-N3-Biotin, thymine cytosine nucleotide or derivative thereof-N3-Atto532-Biotin, and cytosine nucleotide or derivative thereof-N3-CY5;
Combination 22: adenine nucleotide or derivative thereof-N3-Atto647N, guanine nucleotide or derivative thereof-N3-Biotin, thymine cytosine nucleotide or derivative thereof-N3-Biotin, and cytosine nucleotide or derivative thereof-N3-Atto532;
Combination 23: adenine nucleotide or derivative thereof-N3-CY5, guanine nucleotide or derivative thereof-N3-Biotin, thymine nucleotide or derivative thereof-N3-Biotin, and cytosine nucleotide or derivative thereof-N3-Atto532;
Combination 24: adenine nucleotide or derivative thereof-N3-Atto532, guanine nucleotide or derivative thereof-N3-Biotin, thymine cytosine nucleotide or derivative thereof-N3-Biotin, and cytosine nucleotide or derivative thereof-N3-Atto647N;
Combination 25: adenine nucleotide or derivative thereof-N3-Atto532, guanine nucleotide or derivative thereof-N3-Biotin, thymine cytosine nucleotide or derivative thereof-N3-Biotin, and cytosine nucleotide or derivative thereof-N3-CY5.

As a possible implementation of the two-color sequencing kit of the present application, in Combinations 1 to 14, the cleavable group —N3- in any one of the reversible terminators not having the specific binding label is replaced with —N3-SS—, and the cleavable group —N3-SS— in any one of the reversible terminators not having the specific binding label is replaced with —N3-; and/or
in Combinations 1 to 14, —N3-SS— is replaced with —SS—.

As a possible implementation of the two-color sequencing kit of the present application, in Combinations 15 to 25, CY5 and/or Atto647N are/is replaced with GM656 or Alexa Fluor647; and/or Atto532 and/or Alexa Fluor532 are/is replaced with Atto Rho6G or CY3.

As a possible implementation of the two-color sequencing kit of the present application, in Combinations 15 to 25, CY5 and/or Atto647N are/is replaced with one of the blue light dyes.

On the basis of any one of the above embodiments, as a possible implementation of the two-color sequencing method of the present application, the working concentration of the adenine nucleotide or derivative thereof is from 10 nmol/L to 700 nmol/L, the working concentration of the thymine nucleotide or derivative thereof is from 5 nmol/L to 60 nmol/L, the working concentration of the cytosine nucleotide or derivative thereof is from 10 nmol/L to 700 nmol/L, and the working concentration of the guanine nucleotide or derivative thereof is from 0.05 nmol/L to 5 nmol/L.

On the basis of any one of the above embodiments, as a possible implementation of the two-color sequencing method of the present application, the nucleic acid molecules are single-copy nucleic acid templates. The nucleic acid molecules are not amplicons; that is, the nucleic acid molecules are not an amplification cluster formed by amplification on a solid-phase surface.

On the basis of any one of the above embodiments, as a possible implementation of the two-color sequencing method of the present application, the method further comprises:
(f) repeating (a) to (e) one or more times to determine the sequence of the template nucleic acids.

In some examples, the two-color sequencing method comprises:
step 1, providing four types of reversible terminator: a first reversible terminator, a second reversible terminator, a third reversible terminator, and a fourth reversible terminator, the four types of reversible terminator each independently comprising: a nucleotide or derivative thereof, a labeling moiety, and a first cleavable group and/or a second cleavable group linking the nucleotide or derivative thereof and the labeling moiety, the nucleotide or derivative thereof being selected from adenine or derivative thereof, thymine or derivative thereof, cytosine or derivative thereof, or guanine or derivative thereof, and
step 2, allowing the reversible terminators to bind to and extend along nucleic acid template-primer complexes via polymerase chain reactions to obtain extension products.

The allowing the reversible terminators to bind to and extend along nucleic acid template-primer complexes via polymerase chain reactions occurs on a solid-phase carrier, which comprises a plurality of sites, with a nucleic acid template-primer complex being immobilized at each site.

The solid-phase carrier is understood to be a carrier or support on which nucleic acid molecules or nucleic acid templates are immobilized. In some application exemplifications, the solid-phase carrier may also be referred to as a solid-phase substrate, a sequencing chip, or a sequencing biochip. In one example, the solid-phase carrier is a substrate having an array of microwells, and the position where each microwell is located corresponds to a site. Exemplarily, the solid-phase carrier is a nanowell pattern chip, which comprises nanowells arranged in an array, and is used to immobilize nucleic acid template-primer complexes. The nanowells may also be referred to as nanopores, micropores, or holes. In the examples of the present application, the so-called nanowell and nanopore are interchangeable, and refer to a porous structure of a nanoscale size formed on the surface of the solid-phase carrier. The shape of the nanowell or nanopore is not strictly limited, and may be a circle, an ellipse, a triangle, a quadrangle, or another polygon. The nanowell or nanopore may even have no particular shape. In some examples, the nanowell or nanopore is configured to have a cross section having the shape of a symmetrical figure. Exemplarily, the cross section of the nanowell or nanopore is a circle or a regular polygon.

In one example, the solid-phase carrier is a planar substrate, and the surface of the planar substrate comprises a plurality of surface-treated positions. Each of the positions corresponds to a site for immobilizing a nucleic acid template-primer complex. Here, the surface treatment means that the surface is formed with active functional groups capable of linking the nucleic acid template-primer complexes. An example of such a solid phase support is, for example, a random chip.

The solid-phase carrier has a plurality of sites, and there is a certain distance between two adjacent sites. The distance is large enough to enable the optical signals generated by the fluorescent groups at the adjacent sites to be distinguished from each other.

In this example, the solid-phase carrier is immobilized with nucleic acid templates on the surface thereof, and the nucleic acid templates are attached to the surface of the solid-phase carrier. In one embodiment, the solid-phase support is attached with probes on the surface thereof. The probes are covalently connected to molecules/groups on the surface of the solid-phase carrier, and the nucleic acid templates are attached to the surface of the solid-phase carrier via the probes. The probes are oligonucleotides or nucleic acid molecule fragments that can be hybridized with target nucleic acid molecules of interest. In one example, at least a portion of the probes is configured to hybridize with at least a portion of the 3′ end of the nucleic acid templates. In another embodiment, the nucleic acid templates are covalently linked, via one end thereof, to molecules/groups on the surface of the solid-phase carrier, thereby attaching to the surface of the solid-phase carrier. In the examples of the present application, the probes and the nucleic acid templates together form the nucleic acid template-primer complexes.

In specific implementations, the solid-phase carrier has a plurality of sites, and the nucleic acid templates are correspondingly immobilized on the sites of the solid-phase carrier. According to the examples of the present application, ideally, one type of nucleic acid template is immobilized at each site. In some unavoidable circumstances, there may be the case where a very small number of sites are not immobilized with any nucleic acid template or the case where two types of nucleic acid template are immobilized at one site. When the same site contains two different types of nucleic acid template, two detectable signals are generated at the site in one cycle of sequencing. In order to improve the accuracy of sequencing, the sequencing signals of these sites are not used for statistics, and the corresponding sequencing data are eliminated.

In the examples of the present application, each site on the surface of the solid-phase carrier is immobilized with one nucleic acid. In this instance, the two-color sequencing provided in the examples of the present application is single-molecule sequencing. In some other examples, each site on the surface of the solid-phase carrier contains a plurality of amplicons of one nucleic acid molecule; that is, each site contains one cluster of nucleic acid molecules. In this instance, the two-color sequencing provided in the examples of the present application is sequencing provided for the cluster of nucleic acid molecules.

In the examples of the present application, the solid-phase carrier comprises a channel structure capable of communicating one end of the solid-phase carrier with the other end thereof. In this instance, the solution and/or reagents for conducting the polymerase chain reactions in step 2 as well as other solutions that subsequently need to be contacted with the solid-phase carrier can achieve contact by being flown through the surface of the channel structure. In one example, the channel structure on the surface of the solid-phase carrier may be formed on the surface of the solid-phase carrier by means such as etching. The material at a partial region of the surface of the solid-phase carrier is removed by means such as etching, so that a recessed structure is formed at the partial region. The recessed structure extends from one end of the solid-phase carrier to the other end of the solid-phase carrier. The channel structure on the surface of the solid-phase carrier may also be formed by covering a surface of a solid substrate having a certain thickness with a mask material layer, the mask material layer having a pattern corresponding to the channel structure expected to be provided on the surface of the solid-phase carrier. For example, the region where the corresponding recess is located is masked by the mask material layer, and the region other than the recessed region is hollowed out. Then the hollowed-out region of the mask material layer is deposited with a material by means such as chemical deposition, so as to form a material layer having a channel structure on the surface of the solid-phase carrier. The solid-phase carrier and the material layer formed on the surface thereof together constitute the solid-phase carrier. It should be understood that the surface of the solid-phase carrier may have one channel structure, or may have two or even more channel structures, such as 4, 8, or 16 channel structures, although the number of channel structures is not limited thereto. In some examples, the channel structure on the surface of the solid-phase carrier is also referred to as Lane.

Exemplarily, the channel structure has a width of from 5 mm to 15 mm and a height of from 50 m to 200 m. It should be understood that the width and height of the solid-phase carrier may be arbitrarily combined within the above ranges without strict limitation. Exemplarily, when the width of the solid-phase carrier is from 5 mm to 10 mm, the height of the solid-phase carrier may be in the range of from 50 μm to 100 μm, or may be in the range of from 80 μm to 120 μm, or may be in the range of from 100 μm to 150 μm, from 120 μm to 180 μm, from 180 μm to 200 μm, etc. Of course, when the width of the solid-phase carrier is from 10 mm to 15 mm, similarly, the height of the solid-phase carrier may be in the range of from 50 μm to 100 μm, or may be in the range of from 80 μm to 120 μm, or may be in the range of from 100 μm to 150 μm, from 120 μm to 180 μm, from 180 μm to 200 μm, etc.

In step 1 and step 2, four types of nucleotide or analog thereof carrying a labeling moiety and a cleavable group (hereinafter referred to as “reversible terminators”) are added to the surface of the solid-phase carrier under conditions suitable for polymerase chain reactions, so that the reversible terminators can pair with the nucleic acid templates by the principle of base complementary pairing and bind to the nucleic acid template-primer complexes. In the examples of the present application, the four types of reversible terminator are dispersed uniformly via a medium and are in contact with the nucleic acid template-primer complexes. The medium for dispersing the reversible terminators may be a liquid medium, for example, a reagent used for dissolving the four types of reversible terminator, the polymerase, etc. In one example, the four types of reversible terminator and the polymerase are dissolved in the reagent, and the reversible terminators are allowed to come into contact with the nucleic acid template-primer complexes under conditions suitable for polymerization to perform polymerase chain reactions, so that one reversible terminator is incorporated onto a primer strand.

In the examples of the present application, the structures of the reversible terminators are designed in a manner such that the two images collected before and after the cleavable group is cleaved exhibit a regular signal difference for the four types of reversible terminator. The signal differences of the four types of reversible terminator are pooled and analyzed so as to identify the four types of reversible terminator.

Specifically, in the examples of the present application, for the convenience of distinguishing the four types of reversible terminator, the examples of the present application designate the four types of reversible terminator as a first reversible terminator, a second reversible terminator, a third reversible terminator, and a fourth reversible terminator, respectively. The four types of reversible terminator each independently comprise: a nucleotide or derivative thereof, a labeling moiety, and a first cleavable group and/or a second cleavable group linking the nucleotide or derivative thereof and the labeling moiety.

In the examples of the present application, the nucleotide or derivative thereof may be a deoxyribonucleotide or derivative thereof, or may be a ribonucleotide or derivative thereof. When the nucleotide or derivative thereof is a deoxyribonucleotide or derivative thereof, the nucleotides or derivatives thereof for the four types of reversible terminator are adenine deoxyribonucleotide or derivative thereof (denoted as dA), guanine deoxyribonucleotide or derivative thereof (denoted as dG), cytosine deoxyribonucleotide or derivative thereof (denoted as dC), and thymine deoxyribonucleotide or derivative thereof (denoted as dT), respectively; Or the nucleotides or derivatives thereof for the four types of reversible terminator are adenine deoxyribonucleotide or derivative thereof (denoted as dA), guanine deoxyribonucleotides or derivative thereof (denoted as dG), cytosine deoxyribonucleotides or derivative thereof (denoted as dC), and uracil deoxyribonucleotides or derivative thereof (denoted as dU). When the nucleotide or derivative thereof is a ribonucleotide or derivative thereof, the nucleotides or derivatives thereof for the four types of reversible terminator are adenine ribonucleotide or derivative thereof (denoted as A), guanine ribonucleotide or derivative thereof (denoted as G), cytosine ribonucleotide or derivative thereof (denoted as C), and thymine ribonucleotide or derivative thereof (denoted as T), respectively, or the nucleotides or derivatives thereof for the four types of reversible terminator are adenine ribonucleotide or derivative thereof (denoted as A), guanine ribonucleotide or derivative thereof (denoted as G), cytosine ribonucleotide or derivative thereof (denoted as C), and uracil ribonucleotide or derivative thereof (denoted as U), respectively. However, it is worth noting that when the nucleotide or derivative thereof is a ribonucleotide or derivative thereof, the hydroxyl group at the 2′ position of the ribose in the structure of the nucleotide or derivative thereof is modified, so that it cannot react with a phosphate group to form a phosphate ester bond under the polymerization reaction conditions.

In the examples of the present application, the labeling moiety in the reversible terminators includes three scenarios. In the first scenario, the labeling moiety is a fluorescent group capable of being excited under a certain condition to generate a fluorescent signal. In the second scenario, the labeling moiety is a specific binding label capable of specifically binding to a labeling substance. In this instance, the reversible terminator containing the labeling moiety does not generate a fluorescent group, but can generate a fluorescent signal after binding to the labeling substance containing a fluorescent group. In the third scenario, the labeling moiety comprises both a fluorescent group and a specific binding label. In this instance, the reversible terminator containing the labeling moiety can be excited under a certain condition to generate a fluorescent signal, and can generate a composite fluorescent signal after binding to a labeling substance containing a fluorescent group. That is, the labeling moieties in the four types of reversible terminator in the examples of the present application are each independently one of a combination of a fluorescent group and a specific binding label, an independent fluorescent group, and an independent specific binding label.

In one embodiment of the present application, the labeling moiety is a fluorescent group. In this instance, the reversible terminators comprise: a nucleotide or derivative thereof, the fluorescent group, and a first cleavable group and/or a second cleavable group linking the nucleotide or derivative thereof and the fluorescent group.

At least two of the four types of reversible terminator provided in the examples of the present application carry a fluorescent group, and the fluorescent groups carried by the four types of reversible terminator have two distinguishable colors. That is, the fluorescent groups on the four types of reversible terminator are selected from a first fluorescent group and a second fluorescent group which are distinguishable in color, so that the signal difference generated by the four types of reversible terminator before and after the cleavable group is cleaved can realize the identification of the four types of reversible terminator.

In some examples, the fluorescent color of the first fluorescent group is red or green, and the fluorescent color of the second fluorescent group is red or green. Of course, the first fluorescent group and the second fluorescent group are different.

In some embodiments, the first fluorescent group and the second fluorescent group are each independently selected from: one or combinations of more of Atto532, Atto647N, Atto390, Atto Rho6G, Alexa Fluor405, Alexa Fluor532, GM656, Alexa Fluor647, CY5, IF700, ROX, CY3, CY5 rhodamine dye, cyanine dye, coumarin dye, or phycoerythrin;
wherein, the fluorescent group whose fluorescent color is green includes Atto532, Alexa Fluor532, Atto Rho6G, or CY3;
the fluorescent group whose fluorescent color is red includes: Atto647N, CY5, GM656, or Alexa Fluor647;
the fluorescent group whose fluorescent color is blue includes: Atto390, Alexa Fluor405, or coumarin.

In some examples, the combination of the first fluorescent group and the second fluorescent group is a combination of a red fluorescent group and a green fluorescent group. In some other examples, the combination of the first fluorescent group and the second fluorescent group is a combination of a blue fluorescent group and a green fluorescent group.

In another embodiment of the present application, the labeling moiety is a specific binding label. In this instance, the reversible terminators comprise: a nucleotide or derivative thereof, the specific binding label, and a first cleavable group and/or a second cleavable group linking the nucleotide or derivative thereof and the specific binding label. In the examples of the present application, the labeling substance that binds to the specific binding label comprises: a ligand, and a fluorescent group bound to the ligand. In this instance, the reversible terminators may achieve a change in fluorescent signal by means of the specific binding label being bound to the binding substance via a binding reaction.

In some examples of the present application, in each labeling substance, the number of fluorescent groups bound or linked to the ligand is n, wherein n is 1 or 2. Thus, the four types of reversible terminator can exhibit different fluorescent signals before and after the binding reaction. The different fluorescent signals include: the presence or absence of a fluorescent signal, and the difference in the fluorescent color or the fluorescent color combination in the fluorescent signals.

In still another embodiment of the present application, the labeling moiety is a combination of a fluorescent group and a specific binding label. In this instance, the reversible terminators comprise: a nucleotide or derivative thereof, the combination of a fluorescent group and a specific binding label, and a first cleavable group and/or a second cleavable group linking the nucleotide or derivative thereof and the combination. The specific binding label is further capable of binding to a ligand in a labeling substance by a binding reaction so as to bind to another fluorescent group.

In some examples, the labeling moiety is a combination of a fluorescent group and a specific binding label, and in this instance, the color of the fluorescent group carried by the reversible terminator itself is different from the color of the fluorescent group in the labeling substance bound by the specific binding label via the binding reaction. Hence, the composite fluorescent signal generated by the two fluorescent groups formed after the specific binding label is bound to the ligand can be distinguished from the fluorescent signal generated by the fluorescent group before binding, as exemplified by the third reversible terminator provided in Table 2.

In some examples of the present application, when the reversible terminator comprises the combination of a fluorescent group and a specific binding label, the number of fluorescent groups carried by the reversible terminator is 1, and the number of fluorescent groups in the labeling substance bound to the specific binding label via the binding reaction is also 1. It should be understood that the color of the fluorescent group carried by the reversible terminator itself is different from the color of the fluorescent group in the labeling substance bound to the specific binding label via the binding reaction, and the two colors can be distinguished under certain conditions. For example, the colors exhibited by the two fluorescent groups in an acquired image can be identified.

In the examples of the present application, when the labeling moiety comprises a specific binding label, the labeling moiety can bind to a labeling substance via a binding reaction. Specifically, the specific binding label specifically binds to a ligand in the labeling substance.

In some examples of the present application, a combination of the specific binding label and the ligand is selected from at least one of streptavidin (SA)-Biotin combination, Biotin-streptavidin (SA) combination, Biotin-avidin combination, or antibody-antigen combination.

In the examples of the present application, the four types of reversible terminator are each independently provided with one or two cleavable groups. When the reversible terminators are provided with one cleavable group, the cleavable group is removed via one cleavage reaction during the sequencing process; when the reversible terminators are provided with two different cleavable groups, the two cleavable groups are cleaved via two cleavage reactions, respectively.

In the examples of the present application, when the reversible terminators comprise two cleavable groups at the same time, the two cleavable groups are different. In order to distinguish the two cleavable groups, the cleavable group having a stronger cleavability is defined as the second cleavable group, and the first cleavable group has a weaker cleavability than the second cleavable group. It should be understood that, in the examples of the present application, by cleavability it is meant the activity with which a group can be separated from a nucleotide or analog thereof under a certain identical chemical, biological, or physical condition. The mode of separation may be, but not limited to, chemical cleavage, enzyme cleavage, photocleavage, etc., and the extent of cleavability may be reflected by the ability to be cleaved, the length of time required for cleavage, etc. In the examples of the present application, the difference in cleavability between the first cleavable group and the second cleavable group satisfies the following: under a certain identical chemical, biological, or physical condition, the second cleavable group is cleaved from the structure of the nucleotide or derivative thereof, and the first cleavable group remains in the structure of the nucleotide or derivative thereof.

In some examples, the first cleavable group is selected from groups comprising at least one of —N3-, —CH₂F, —C(O)NHBu, —CHF₂, —C(O)OH, —C₆ and/or —ArC₆, or disulfide bond. In some examples, the first cleavable group is selected from at least one of —N₃—, —CH₂F, —C(O)NHBu, —CHF₂, —C(O)OH, —C₆ and/or —ArC₆, or disulfide bond. In some more specific examples, the second cleavable group is selected from groups comprising disulfide bond, and further, the second cleavable group is disulfide bond (—SS—).

In one embodiment of the present application, the structures of the four types of reversible terminator satisfy the following:
the structure of the first reversible terminator is: first nucleotide or derivative thereof-first cleavable group-first fluorescent group;
the structure of the second reversible terminator is one of the following scenarios: second nucleotide or derivative thereof-first cleavable group-second cleavable group-first fluorescent group or second fluorescent group; or second nucleotide or derivative thereof-second cleavable group-first fluorescent group or second fluorescent group;
the structure of the third reversible terminator is one of the following scenarios: second nucleotide or derivative thereof-first cleavable group-specific binding label; or third nucleotide or derivative thereof-first cleavable group-first fluorescent group or second fluorescent group-specific binding label;
the structure of the second reversible terminator is: fourth nucleotide or derivative thereof-first cleavable group-second fluorescent group;
the second reversible terminator and the third reversible terminator do not comprise the specific binding label at the same time;
wherein, the first nucleotide or derivative thereof, the second nucleotide or derivative thereof, the third nucleotide or derivative thereof, and the fourth nucleotide or derivative thereof are selected from one of adenine or derivative thereof, thymine or derivative thereof, cytosine or derivative thereof, or guanine or derivative thereof, and the first nucleotide or derivative thereof, the second nucleotide or derivative thereof, the third nucleotide or derivative thereof, and the fourth nucleotide or derivative thereof are different from each other.

In this instance, the labeling moieties of the reversible terminators in the examples of the present application can realize the signal difference of the four types of reversible terminator by the change in fluorescent signal before and after the second cleavable group is cleaved and the change in fluorescent signal before and after the specific binding label binds to the labeling substance, thereby identifying the type of the four types of reversible terminator. When the reversible terminator comprises only one type of cleavable group, the signal difference of the four types of reversible terminator is realized by the change in fluorescence signal before and after the specific binding label binds to the labeling substance, thereby identifying the type of the four types of reversible terminator.

In the specific scenarios shown in Tables 1 to 5 of the above Scheme 1, the cleavable group includes a first cleavable group, a second cleavable group, or a combination of a first cleavable group and a second cleavable group.

Exemplarily, the reversible terminators include at least one of the following combinations:
Combination 1: adenine nucleotide or derivative thereof-N3-Atto647N, guanine nucleotide or derivative thereof-N3-SS-Atto647N, thymine nucleotide or derivative thereof-N3-Biotin, and cytosine nucleotide or derivative thereof-N3-Atto532;
Combination 2: adenine nucleotide or derivative thereof-N3-CY5, guanine nucleotide or derivative thereof-N3-SS-Atto647N, thymine nucleotide or derivative thereof-N3-Biotin, and cytosine nucleotide or derivative thereof-N3-Atto532;
Combination 3: adenine nucleotide or derivative thereof-N3-CY5, guanine nucleotide or derivative thereof-N3-SS-CY5, thymine nucleotide or derivative thereof-N3-Biotin, and cytosine nucleotide or derivative thereof-N3-Atto532;
Combination 4: adenine nucleotide or derivative thereof-N3-Atto647N, guanine nucleotide or derivative thereof-N3-SS-CY5, thymine nucleotide or derivative thereof-N3-Biotin, and cytosine nucleotide or derivative thereof-N3-Atto532;
Combination 5: adenine nucleotide or derivative thereof-N3-Atto647N, guanine nucleotide or derivative thereof-N3-SS-Atto647N, thymine nucleotide or derivative thereof-N3-Atto532-Biotin, and cytosine nucleotide or derivative thereof-N3-Atto532;
Combination 6: adenine nucleotide or derivative thereof-N3-Atto647N, guanine nucleotide or derivative thereof-N3-SS-CY5, cytosine nucleotide or derivative thereof-N3-Atto532-Biotin, and cytosine nucleotide or derivative thereof-N3-Atto532;
Combination 7: adenine nucleotide or derivative thereof-N3-CY5, guanine nucleotide or derivative thereof-N3-SS-Atto647N, thymine nucleotide or derivative thereof-N3-Atto532-Biotin, and cytosine nucleotide or derivative thereof-N3-Atto532;
Combination 8: adenine nucleotide or derivative thereof-N3-CY5, guanine nucleotide or derivative thereof-N3-SS-CY5, thymine nucleotide or derivative thereof-N3-Atto532-Biotin, and cytosine nucleotide or derivative thereof-N3-Atto532;
Combination 9: adenine nucleotide or derivative thereof-N3-Atto647N, guanine nucleotide or derivative thereof-N3-SS-Atto532, thymine nucleotide or derivative thereof-N3-Biotin, and cytosine nucleotide or derivative thereof-N3-Atto532;
Combination 10: adenine nucleotide or derivative thereof-N3-CY5, guanine nucleotide or derivative thereof-N3-SS-Atto532, thymine nucleotide or derivative thereof-N3-Biotin, and cytosine nucleotide or derivative thereof-N3-Atto532;
Combination 11: adenine nucleotide or derivative thereof-N3-Atto532, guanine nucleotide or derivative thereof-N3-SS-Atto532, cytosine nucleotide or derivative thereof-N3-Biotin, and cytosine nucleotide or derivative thereof-N3-Atto647N;
Combination 12: adenine nucleotide or derivative thereof-N3-Atto532, guanine nucleotide or derivative thereof-N3-SS-Atto532, thymine nucleotide or derivative thereof-N3-Biotin, and cytosine nucleotide or derivative thereof-N3-CY5;
Combination 13: adenine nucleotide or derivative thereof-N3-Atto532, guanine nucleotide or derivative thereof-N3-SS-Atto532, cytosine nucleotide or derivative thereof-N3-Atto647N-Biotin, and cytosine nucleotide or derivative thereof-N3-Atto647N;
Combination 14: adenine nucleotide or derivative thereof-N3-Atto532, guanine nucleotide or derivative thereof-N3-SS-Atto532, thymine cytosine nucleotide or derivative thereof-N3-CY5-Biotin, and cytosine nucleotide or derivative thereof-N3-CY5.

In the examples of the present application, other combinations of the four types of reversible terminator can be obtained by adjusting the type of the cleavable group in Combinations 1 to 14. In some examples, new combinations can be obtained by adjusting the type of the cleavable group in Combinations 1 to 14, specifically as follows:
in Combinations 1 to 14, the cleavable group —N3- in any one of the reversible terminators not having the specific binding label is replaced with —N3-SS—, and the cleavable group —N3-SS— in any one of the reversible terminators not having the specific binding label is replaced with —N3-; and/or
in Combinations 1 to 14, —N3-SS— is replaced with —SS—.

In the examples of the present application, other combinations of the four types of reversible terminator can be obtained by adjusting the type of the fluorescent group in Combinations 1 to 14 before and after adjusting the cleavable group. In some examples, the type of the fluorescent group in Combinations 1 to 14 is adjusted, such that in Combinations 1 to 14, CY5 and/or Atto647N are/is replaced with GM656 or Alexa Fluor647. In some examples, the type of the fluorescent group in Combinations 1-14 is adjusted, such that Atto532 and/or Alexa Fluor532 are/is replaced with Atto Rho6G or CY3. In some examples, the type of the fluorescent group in Combinations 1-14 is adjusted, such that CY5 and/or Atto647N are/is replaced with GM656 or Alexa Fluor647, and Atto532 and/or Alexa Fluor532 are/is replaced with Atto Rho6G or CY3. In some examples, the type of the fluorescent group in Combinations 1 to 14 is adjusted, such that CY5 and/or Atto647N are/is replaced with one of the blue light dyes.

In some other examples of the two-color sequencing method of the examples of the present application, the four types of reversible terminator are provided with different cleavable groups and/or different labeling moieties, and there may be one cleavage reaction, or two cleavage reactions. When there is one cleavage reaction, the structural difference of the cleavable group may further distinguish the four types of reversible terminator by means of a difference in specific reactions, specifically including the connection mode and connection number of fluorescent groups, and specifically Scheme 2 of the two-color sequencing method of the examples of the present application. Exemplary signal situations and steps of Scheme 2 are shown in Tables 6 to 10.

In Scheme 2, a binding reaction is performed before specific cleavage occurs; The occurrence of the binding reaction is related to the composition of the detectable group in the reversible terminator.

In the specific Example 2 of the present application, the specific implementations of Scheme 2 can be classified into two types. The specific scenario of the first type is that the second signal is no bright spot, and the exemplary signal situations and detection steps are shown in Tables 6 to 10 (Scheme 2a to c). The specific scenario of the second type includes that the second signal and the third signal in the detection of step 3 are no bright spot (Scheme 2d and e).

No bright spot is specifically reflected in the structure of the reversible terminators. The reversible terminators only have a specific binding label, and the fluorescent group thereof is linked to a ligand. The reversible terminators can specifically be distinguished after the sixth and seventh signals are obtained in step 5.

In both situations of Scheme 2, the number of fluorescent groups to which the labeling substance is bound, provided with, or attached is n, wherein n is 1 or 2; or the number of fluorescent groups to which the specific binding label is bound, provided with, or attached is m, wherein m is 1 or 2; This ensures that a first subset and a second subset have different fluorescence situations. The different fluorescence situations include presence or absence of fluorescence, different fluorescence colors, and different types of fluorescence color.

After step 3, exciting the extension products and collecting fluorescent signals therefrom to obtain a first image, at least a subset of the four types of reversible terminator (hereinafter referred to as a first subset) can be distinguished from the other subset of reversible terminators (hereinafter referred to as a second subset). After performing a binding reaction, and after step 5, exciting the reaction products and collecting fluorescent signals therefrom to obtain a second image, the reversible terminators in the first subset of the four types of reversible terminator can be distinguished from each other, and the reversible terminators in the second subset can also be distinguished from each other. Hence, finally, the signals of the four types of reversible terminator with two-color fluorescence are distinguished one by one.

As the first specific scenario of Scheme 2 (Scheme 2a to 2e), the structures of the four types of reversible terminator include at least one of the following combinations:
in one embodiment of the present application, the cleavable groups in the four types of reversible terminator are the same and are any one of the first cleavable group or the second cleavable group;
the first signal, the second signal, the third signal, the sixth signal, and the seventh signal satisfy condition 3) or condition 4), and the structures of the four types of reversible terminator satisfy the following:
the structure of the first reversible terminator is: first nucleotide or derivative thereof-first cleavable group or second cleavable group-first fluorescent group;
the structure of the second reversible terminator is: second nucleotide or derivative thereof-first cleavable group or second cleavable group-specific binding label; or second nucleotide or derivative thereof-first cleavable group or second cleavable group-first fluorescent group or second fluorescent group-specific binding label;
the structure of the third reversible terminator is: third nucleotide or derivative thereof-first cleavable group or second cleavable group-specific binding label; or third nucleotide or derivative thereof-first cleavable group or second cleavable group-second fluorescent group or first fluorescent group-specific binding label;
the structure of the second reversible terminator is: fourth nucleotide or derivative thereof-first cleavable group or second cleavable group-second fluorescent group;
wherein, the first nucleotide or derivative thereof, the second nucleotide or derivative thereof, the third nucleotide or derivative thereof, and the fourth nucleotide or derivative thereof are selected from one of adenine or derivative thereof, thymine or derivative thereof, cytosine or derivative thereof, or guanine or derivative thereof, and the first nucleotide or derivative thereof, the second nucleotide or derivative thereof, the third nucleotide or derivative thereof, and the fourth nucleotide or derivative thereof are different from each other.

Exemplarily, the reversible terminators include at least one of the following combinations:
Combination 15: adenine nucleotide or derivative thereof-N3-Atto647N, guanine nucleotide or derivative thereof-N3-Biotin, thymine nucleotide or derivative thereof-N3-Atto532-Biotin, and cytosine nucleotide or derivative thereof-N3-Atto532;
Combination 16: adenine nucleotide or derivative thereof-N3-CY5, guanine nucleotide or derivative thereof-N3-Biotin, thymine nucleotide or derivative thereof-N3-Atto532-Biotin, and cytosine nucleotide or derivative thereof-N3-Atto532;
Combination 17: adenine nucleotide or derivative thereof-N3-Atto647N, guanine nucleotide or derivative thereof-N3-Biotin, thymine cytosine nucleotide or derivative thereof-N3-Atto647N-Biotin, and cytosine nucleotide or derivative thereof-N3-Atto532;
Combination 18: adenine nucleotide or derivative thereof-N3-CY5, guanine nucleotide or derivative thereof-N3-Biotin, thymine nucleotide or derivative thereof-N3-Atto647N-Biotin, and cytosine nucleotide or derivative thereof-N3-Atto532;
Combination 19: adenine nucleotide or derivative thereof-N3-Atto647N, guanine nucleotide or derivative thereof-N3-Biotin, thymine nucleotide or derivative thereof-N3-CY5-Biotin, and cytosine nucleotide or derivative thereof-N3-Atto532;
Combination 20: adenine nucleotide or derivative thereof-N3-Atto532, guanine nucleotide or derivative thereof-N3-Biotin, thymine cytosine nucleotide or derivative thereof-N3-Atto532-Biotin, and cytosine nucleotide or derivative thereof-N3-Atto647N;
Combination 21: adenine nucleotide or derivative thereof-N3-Atto532, guanine nucleotide or derivative thereof-N3-Biotin, thymine cytosine nucleotide or derivative thereof-N3-Atto532-Biotin, and cytosine nucleotide or derivative thereof-N3-CY5;
Combination 22: adenine nucleotide or derivative thereof-N3-Atto647N, guanine nucleotide or derivative thereof-N3-Biotin, thymine cytosine nucleotide or derivative thereof-N3-Biotin, and cytosine nucleotide or derivative thereof-N3-Atto532;
Combination 23: adenine nucleotide or derivative thereof-N3-CY5, guanine nucleotide or derivative thereof-N3-Biotin, thymine nucleotide or derivative thereof-N3-Biotin, and cytosine nucleotide or derivative thereof-N3-Atto532;
Combination 24: adenine nucleotide or derivative thereof-N3-Atto532, guanine nucleotide or derivative thereof-N3-Biotin, thymine cytosine nucleotide or derivative thereof-N3-Biotin, and cytosine nucleotide or derivative thereof-N3-Atto647N;
Combination 25: adenine nucleotide or derivative thereof-N3-Atto532, guanine nucleotide or derivative thereof-N3-Biotin, thymine cytosine nucleotide or derivative thereof-N3-Biotin, and cytosine nucleotide or derivative thereof-N3-CY5.

In the examples of the present application, other combinations of the four types of reversible terminator can be obtained by adjusting the type of the cleavable group in Combinations 15 to 25. In some examples, adjusting the type of the cleavable group in Combinations 15 to 25 by replacing the cleavable group —N3- with —SS— can form new combinations of reversible terminators.

In the examples of the present application, other combinations of the four types of reversible terminator can be obtained by adjusting the type of the fluorescent group in Combinations 15 to 25 before and after adjusting the cleavable group. In some examples, the type of the fluorescent group in Combinations 15 to 25 is adjusted, such that in Combinations 15 to 25, CY5 and/or Atto647N are/is replaced with GM656 or Alexa Fluor647. In some examples, the type of fluorescent group in Combinations 15 to 25 is adjusted, such that in Combinations 15 to 25, Atto532 and/or Alexa Fluor532 are/is replaced with Atto Rho6G or CY3. In some examples, the type of fluorescent group in Combinations 15 to 25 is adjusted, such that in Combinations 15 to 25, CY5 and/or Atto647N are/is replaced with GM656 or Alexa Fluor647; and Atto532 and/or Alexa Fluor532 are/is replaced with Atto Rho6G or CY3. In some examples, the type of fluorescent group in Combinations 15 to 25 is adjusted, such that in Combinations 15 to 25, CY5 and/or Atto647N are/is replaced with one of the blue light dyes.

In some examples, the final concentration of each reversible terminator is from 0.05 nmol/L to 2000 nmol/L. It should be understood that the final concentrations or use concentrations of the reversible terminators referred to in the examples of the present application mean the concentrations of the reversible terminators when a solution containing the reversible terminators is in contact with the nucleic acid template-primer complexes. Exemplarily, a reaction solution containing the reversible terminators is introduced into the solid-phase carrier, and the concentrations of the reversible terminators in the reaction solution are the final concentrations of the reversible terminators. In one example, the step involves pumping different reagents from different kits, mixing the reagents to form a mixed solution, and flowing the mixed solution onto the surface of the solid-phase carrier. In this instance, the final concentrations of the reversible terminators should be the concentrations of the reversible terminators in the mixed solution. In the examples of the present application, the final concentrations of the reversible terminators, set under the conditions for the polymerase chain reactions, are amenable to adjustment within the above ranges such that under the conditions for the polymerase chain reactions, absorption of the reversible terminators occurring in regions other than surface sites of the solid-phase carrier is reduced, thereby reducing the non-specific adsorption thus formed. The fluorescent groups in the reversible terminators at the positions where such non-specific adsorption occurs are excited in step 3 to generate optical signals, which increases the background noise in the image obtained by imaging, interferes with the identification of the optical signals of the fluorescent groups at the sites, and hence affects the accuracy of base identification.

In the examples of the present application, the final concentrations of the reversible terminators can be adjusted within the range of from 0.05 nmol/L to 2000 nmol/L according to the type of the reversible terminators, the polymerase concentration in the reaction system, etc.

As one scenario of the reversible terminators in the examples of the present application, the nucleotide or derivative thereof in the reversible terminators includes adenine nucleotide or derivative thereof. More specifically, in the specific examples of the present application, the structure of the corresponding reversible terminator is dA-N3-Atto647N. When the reaction system for the polymerase chain reactions in step 2 comprises dA-N3-Atto647N, the final concentration of dA-N3-Atto647N is from 1 nmol/L to 700 nmol/L, and may specifically be 1 nmol/L, 2 nmol/L, 3 nmol/L, 4 nmol/L, 5 nmol/L, 6 nmol/L, 7 nmol/L, 8 nmol/L, 9 nmol/L, 10 nmol/L, 15 nmol/L, 20 nmol/L, 30 nmol/L, 40 nmol/L, 50 nmol/L, 60 nmol/L, 70 nmol/L, 80 nmol/L, 90 nmol/L, 100 nmol/L, 125 nmol/L, 150 nmol/L, 175 nmol/L, 200 nmol/L, 225 nmol/L, 250 nmol/L, 275 nmol/L, 300 nmol/L, 325 nmol/L, 350 nmol/L, 375 nmol/L, 400 nmol/L, 425 nmol/L, 450 nmol/L, 475 nmol/L, 500 nmol/L, 525 nmol/L, 550 nmol/L, 575 nmol/L, 600 nmol/L, 625 nmol/L, 650 nmol/L, 675 nmol/L, 700 nmol/L, etc.

As another scenario of the reversible terminators in the examples of the present application, the nucleotide or derivative thereof in the reversible terminators includes guanine nucleotide or derivative thereof. More specifically, in the specific examples of the present application, when the structure of the corresponding reversible terminator is dG-SS-Atto647N or dG-N3-SS-CY5, the final concentration of dG-SS-Atto647N or dG-N3-SS-CY5 is from 0.05 nmol/L to 5 nmol/L, and may specifically be 0.05 nmol/L, 0.06 nmol/L, 0.07 nmol/L, 0.08 nmol/L, 0.09 nmol/L, 0.1 nmol/L, 0.15 nmol/L, 0.25 nmol/L, 0.35 nmol/L, 0.45 nmol/L, 0.55 nmol/L, 0.65 nmol/L, 0.75 nmol/L, 0.85 nmol/L, 0.95 nmol/L, 1 nmol/L, 1.15 nmol/L, 1.25 nmol/L, 1.35 nmol/L, 1.45 nmol/L, 1.55 nmol/L, 1.65 nmol/L, 1.75 nmol/L, 1.85 nmol/L, 1.95 nmol/L, 2 nmol/L, 2.15 nmol/L, 2.25 nmol/L, 2.35 nmol/L, 2.45 nmol/L, 2.55 nmol/L, 2.65 nmol/L, 2.75 nmol/L, 2.85 nmol/L, 2.95 nmol/L, 3 nmol/L, 3.15 nmol/L, 3.25 nmol/L, 3.35 nmol/L, 3.45 nmol/L, 3.55 nmol/L, 3.65 nmol/L, 3.75 nmol/L, 3.85 nmol/L, 3.95 nmol/L, 4 nmol/L, 4.15 nmol/L, 4.25 nmol/L, 4.35 nmol/L, 4.45 nmol/L, 4.55 nmol/L, 4.65 nmol/L, 4.75 nmol/L, 4.85 nmol/L, 4.95 nmol/L, 5 nmol/L, etc.

As another scenario of the reversible terminators in the examples of the present application, the nucleotide or derivative thereof in the reversible terminators includes cytosine nucleotide or derivative thereof. More specifically, in the specific examples of the present application, when the structure of the corresponding reversible terminator is dC-N3-Atto532 or dC-N3-Alexa Fluor532, the final concentration of dC-N3-Atto532 or dC-N3-Alexa Fluor532 is from 10 nmol/L to 700 nmol/L, and may specifically be 10 nmol/L, 15 nmol/L, 20 nmol/L, 30 nmol/L, 40 nmol/L, 50 nmol/L, 60 nmol/L, 70 nmol/L, 80 nmol/L, 90 nmol/L, 100 nmol/L, 125 nmol/L, 150 nmol/L, 175 nmol/L, 200 nmol/L, 225 nmol/L, 250 nmol/L, 275 nmol/L, 300 nmol/L, 325 nmol/L, 350 nmol/L, 375 nmol/L, 400 nmol/L, 425 nmol/L, 450 nmol/L, 475 nmol/L, 500 nmol/L, 525 nmol/L, 550 nmol/L, 575 nmol/L, 600 nmol/L, 625 nmol/L, 650 nmol/L, 675 nmol/L, 700 nmol/L, etc.

As another scenario of the reversible terminators in the examples of the present application, the nucleotide or derivative thereof in the reversible terminators includes thymine nucleotide or derivative thereof. More specifically, in the specific examples of the present application, when the structure of the corresponding reversible terminator is dT-N3-Biotin or dT-N3-Atto532-Biotin, the final concentration of dT-SS-Atto532 or dT-N3-SS-Atto532 is from 5 nmol/L to 60 nmol/L, and may specifically 5 nmol/L, 6 nmol/L, 7 nmol/L, 8 nmol/L, 9 nmol/L, 10 nmol/L, 12 nmol/L, 14 nmol/L, 16 nmol/L, 18 nmol/L, 20 nmol/L, 22 nmol/L, 24 nmol/L, 26 nmol/L, 28 nmol/L, 30 nmol/L, 32 nmol/L, 34 nmol/L, 36 nmol/L, 38 nmol/L, 40 nmol/L, 42 nmol/L, 44 nmol/L, 46 nmol/L, 48 nmol/L, 50 nmol/L, 52 nmol/L, 54 nmol/L, 56 nmol/L, 58 nmol/L, 60 nmol/L, etc.

In the examples of the present application, the polymerase for the reversible terminators to undergo a polymerization reaction may be a DNA polymerase or an RNA polymerase, or may include both a DNA polymerase and an RNA polymerase. In some examples, the polymerase is a DNA polymerase, including but not limited to one or more of 9°Nase, Pfu, KOD1, and MMS2.

The final concentration of the polymerase referred to in the examples of the present application means the concentration of the polymerase when a solution containing the polymerase is in contact with the solid-phase carrier. Exemplarily, a reaction solution containing the polymerase is introduced into the solid-phase carrier, and the concentration of the polymerase in the reaction solution is the final concentration of the polymerase. In one embodiment, the step involves pumping different reagents from different kits, mixing the reagents to form a mixed solution, and flowing the mixed solution onto the surface of the solid-phase carrier. In this instance, the final concentration of the polymerase should be the concentration of the polymerase in the mixed solution. In the examples of the present application, the final concentration of the polymerase is set to be from 0.005 mg/mL to 0.02 mg/mL, such that under the conditions for the polymerase chain reactions, absorption of the reversible terminators occurring in regions other than surface sites of the solid-phase carrier is reduced, thereby reducing the non-specific adsorption thus formed. The fluorescent groups in the reversible terminators at the positions where such non-specific adsorption occurs are excited in step 3 and step 5 to generate optical signals, which increases the background noise in the image obtained by imaging, interferes with the identification of the optical signals at the sites, and hence affects the accuracy of base identification.

Exemplarily, the final concentration of the polymerase is specifically 0.005 mg/mL, 0.006 mg/mL, 0.007 mg/mL, 0.008 mg/mL, 0.009 mg/mL, 0.01 mg/mL, 0.011 mg/mL, 0.012 mg/mL, 0.013 mg/mL, 0.014 mg/mL, 0.015 mg/mL, 0.016 mg/mL, 0.017 mg/mL, 0.018 mg/mL, 0.019 mg/mL, 0.02 mg/mL, etc.

In some examples, the polymerase chain reactions are performed under the condition of a pH of from 8.0 to 9.0. Therefore, by regulating the concentrations of the reversible terminators participating in the polymerase chain reactions, the concentration of the polymerase, and the pH condition of the reaction system, absorption of the reversible terminators occurring in regions other than surface sites of the solid-phase carrier can be reduced, thereby reducing the non-specific adsorption thus formed, such that the background noise in the images obtained in step 3 and step 5 can be reduced, and the accuracy of base recognition can be improved.

In the examples of the present application, the reaction system of the polymerase chain reactions further comprises a solvent for dissolving the reversible terminators and the polymerase. The solvent includes a buffer, which may be such as Tris buffer, glycine, ethanolamine, tetraethylethylenediamine, tetramethylethylenediamine, N-butyldiethanolamine, diethylaminoethanol, Hepes buffer, or N,N-dihydroxyethylglycine.

In some examples, the concentration of the buffer is from 0.01 mol/L to 2 mol/L. The pH environment provided when the concentration of the buffer is from 0.01 mol/L to 2 mol/L is beneficial to improving the homogeneity and stability of the reduction reaction. Exemplarily, the concentration of the buffer in the mixed solution is specifically 10 mmol/L, 15 mmol/L, 20 mmol/L, 25 mmol/L, 30 mmol/L, 35 mmol/L, 40 mmol/L, 45 mmol/L, 50 mmol/L, 55 mmol/L, 60 mmol/L, 65 mmol/L, 70 mmol/L, 75 mmol/L, 80 mmol/L, 85 mmol/L, 90 mmol/L, 95 mmol/L, 100 mmol/L, 105 mmol/L, 110 mmol/L, 115 mmol/L, 120 mmol/L, 125 mmol/L, 130 mmol/L, 135 mmol/L, 140 mmol/L, 145 mmol/L, 150 mmol/L, 155 mmol/L, 160 mmol/L, 165 mmol/L, 170 mmol/L, 175 mmol/L, 180 mmol/L, 0.2 mol/L, 0.3 mol/L, 0.4 mol/L, 0.5 mol/L, 0.6 mol/L, 0.7 mol/L, 0.8 mol/L, 0.9 mol/L, 1.0 mol/L, 1.1 mol/L, 1.2 mol/L, 1.3 mol/L, 1.4 mol/L, 1.5 mol/L, 1.6 mol/L, 1.7 mol/L, 1.8 mol/L, 1.9 mol/L, 2.0 mol/L, etc.

Binding of the four types of reversible terminator to the nucleic acid template-primer complexes is achieved via the polymerase chain reactions. The reversible terminators that are not bound to the nucleic acid template-primer complexes are removed by washing, and now the sites of the solid-phase carrier where the nucleic acid template-primers are bound carry a fluorescent group or does not carry a fluorescent group. Since the reversible terminators added at the same time have different fluorescent groups and cleavable groups, the types of the reversible terminators incorporated into the extension products of step 2 can be distinguished by performing subsequent step 3, step 4, and step 5.

Step 3 involves exciting the extension products and collecting fluorescent signals therefrom to obtain a first image, the first image comprising a first signal, a second signal, a third signal, and a fourth signal from the first reversible terminator, the second reversible terminator, the third reversible terminator, and the fourth reversible terminator, respectively.

It should be understood that the wavelength of the excitation light required to generate an optical signal is different for different fluorescent groups. In some examples, the fluorescent group of the reversible terminators added in step 2 includes a first fluorescent group and a second fluorescent group, and the excitation wavelength of the first fluorescent group differs from the excitation wavelength of the second fluorescent group by at least 50 nm, so as to increase the difference between the optical signal generated by the first fluorescent group and the optical signal generated by the second fluorescent group. The greater the difference between the excitation wavelength of the first fluorescent group and the excitation wavelength of the second fluorescent group, the more obvious the difference between the optical signal generated by the first fluorescent group and the optical signal generated by the second fluorescent group, and the easier the two fluorescent groups are to be identified.

In this step, an exposure treatment is performed after the fluorescent groups are excited to generate fluorescent signals. In some examples, the time of the exposure treatment is from 100 ms to 400 ms. By controlling the exposure time, the difference in the intensity of the optical signals generated by the fluorescent groups can be increased to a certain extent, thereby facilitating the identification of the type of the reversible terminator introduced at each site and improving the accuracy of base identification. For example, the time of the exposure treatment is 100 ms, 120 ms, 150 ms, 180 ms, 200 ms, 220 ms, 250 ms, 280 ms, 300 ms, 320 ms, 350 ms, 380 ms, 400 ms, etc. It should be understood that the time of the exposure treatment may be adjusted according to the type of the fluorescent groups so as to achieve the purpose of increasing the difference in the intensity of the optical signals generated by the fluorescent groups. In one example, the first fluorescent group is a red light-excitable fluorescent group, and the exposure time of the red light-excitable group is from 100 ms to 400 ms, such as 100 ms, 120 ms, 150 ms, 180 ms, 200 ms, 220 ms, 250 ms, 280 ms, 300 ms, 320 ms, 350 ms, 380 ms, or 400 ms; the second fluorescent group is a green light-excitable fluorescent group, and the exposure time of the green light-excitable group is from 50 ms to 100 ms, such as 50 ms, 60 ms, 70 ms, 80 ms, 90 ms, or 100 ms.

After the fluorescent groups are excited to generate optical signals, the extension products are imaged to obtain a first image. The first image comprises a first signal, a second signal, a third signal, and a fourth signal from the first reversible terminator, the second reversible terminator, the third reversible terminator, and the fourth reversible terminator, respectively. In the examples of the present application, the first signal, the second signal, the third signal, and the fourth signal are each independently one of a first bright spot, a second bright spot, a third bright spot, and no bright spot, wherein the no bright spot indicates absence of a fluorescent signal, the first bright spot indicates a first fluorescent color feature, the second bright spot indicates a second fluorescent color feature, and the third bright spot indicates both the first fluorescent color feature and the second fluorescent color feature. It can be understood that the fluorescence color feature refers to a feature that can represent the fluorescence color, including the fluorescence color itself, and other indexes that can represent the fluorescence color. In the examples where the fluorescent group in the four types of reversible terminator comprises the first fluorescent group and/or the second fluorescent group, the first fluorescent color feature corresponds to the color of the fluorescence generated by the first fluorescent group when excited, and the second fluorescent color feature corresponds to the color of the fluorescence generated by the second fluorescent group when excited.

In the examples of the present application, among the fluorescent signals generated by the four types of reversible terminator, the first signal is different from the fourth signal, the first signal and the second signal may be the same or different, the third signal and the fourth signal may be the same or different, and the second signal and the third signal may be the same or different. Thus, a subset of the four types of reversible terminator are distinguished from the other subset. See Tables 1 to 10.

Step 4 involves, after obtaining the first image, performing a first reaction and a second reaction on the extension products, or performing a second reaction on the extension products, to obtain reaction products.

In this step, the first reaction is a reaction in which the second cleavable group in the reversible terminator structure is cleaved. Exemplarily, suitable cleavage methods include, but are not limited to, chemical cleavage, enzymatic cleavage, and photocleavage. It should be noted that in this step, in the step of cleavage via the first reaction, the second cleavable group has higher cleavability than the first cleavable group, and the condition for the cleavage of the second cleavable group in the extension products does not achieve cleavage of the first cleavable group in the reversible terminators. Thus, this step enables cleavage of the labeling moiety in the reversible terminators having the second cleavable group from the reversible terminators together with the second cleavable group. Since the first cleavable group in the reversible terminators having the first cleavable group is retained, when the reversible terminators comprise the first cleavable group, the labeling moiety in these reversible terminators is also retained. After cleaving the second cleavable group via the first reaction, at least a subset of the four types of reversible terminator can be distinguished from the other subset of reversible terminators.

In this step, the second reaction is a reaction in which the specific binding label binds to the labeling substance. After contacting the labeling substance via the second reaction alone, at least a subset of the four types of reversible terminator can be distinguished from the other subset of reversible terminators. When step 4 includes both the first reaction and the second reaction, at least a subset of the four types of reversible terminator can be distinguished from the other subset of reversible terminators by virtue of the change of the fluorescent groups in the reversible terminators after the two reactions. In some examples, the time of the second reaction is greater than or equal to 20 s. In some examples, the time of the second reaction is from 20 s to 50 s. Exemplarily, the time of the second reaction is 20 s, 25 s, 30 s, 35 s, 40 s, 45 s, 50 s, etc.

The reaction(s) in step 4 is/are determined by the structure of the reversible terminators.

In some examples of the present application, in the case that among the four types of reversible terminator provided in step 2, the structure of at least one reversible terminator comprises the first cleavable group, the structure of at least one reversible terminator comprises the second cleavable group, and the structure of at least one reversible terminator comprises the specific binding label, step 4 involves, after obtaining the first image, performing the first reaction and the second reaction on the extension products to obtain reaction products (as in Scheme 1). Via the first reaction and the second reaction, the reversible terminators that are not distinguished in step 3 become differentiated in the acquired fluorescence image, thus finally realizing the identification of the four types of reversible terminator.

In some other examples of the present application, in the case that the structure of the four types of reversible terminator provided in step 2 has a single cleavable group, that is, the four types of reversible terminator comprises only the first cleavable group or the second cleavable group, and the structure of at least one reversible terminator comprises the specific binding label, step 4 involves, after obtaining the first image, performing the second reaction on the extension products to obtain reaction products (as in Scheme 2). Via the second reaction, the reversible terminators that are not distinguished in step 3 become differentiated in the acquired fluorescence image, thus finally realizing the identification of the four types of reversible terminator.

In some examples, the second cleavable group is a group comprising disulfide bond, the first cleavable group is selected from groups comprising at least one of —N3-, —CH₂F, —C(O)NHBu, —CHF₂, —C(O)OH, —C₆ and/or —ArC₆. In this instance, the second cleavable group in the reversible terminators is cleaved by adding a cleavage reagent comprising a reducing agent, and the first cleavable group in the reversible terminators is retained.

Exemplarily, the reducing agent for cleaving the second cleavable group includes, but is not limited to, any one or more of DTT, glutathione, and β-mercaptoethanol.

In some examples, the concentration of the reducing agent is from 10 mmol/L to 100 mmol/L, and specifically, the concentration of the reducing agent may be 10 mmol/L, 15 mmol/L, 20 mmol/L, 25 mmol/L, 30 mmol/L, 35 mmol/L, 40 mmol/L, 45 mmol/L, 50 mmol/L, 55 mmol/L, 60 mmol/L, 65 mmol/L, 70 mmol/L, 75 mmol/L, 80 mmol/L, 85 mmol/L, 90 mmol/L, 95 mmol/L, 100 mmol/L, etc.

In one example, glutathione is used as the reducing agent to cleave the second cleavable group, and the use concentration of glutathione is from 10 mmol/L to 100 mmol/L. Exemplarily, the concentration of glutathione may be 10 mmol/L, 15 mmol/L, 20 mmol/L, 25 mmol/L, 30 mmol/L, 35 mmol/L, 40 mmol/L, 45 mmol/L, 50 mmol/L, 55 mmol/L, 60 mmol/L, 65 mmol/L, 70 mmol/L, 75 mmol/L, 80 mmol/L, 85 mmol/L, 90 mmol/L, 95 mmol/L, 100 mmol/L, etc.

In one embodiment, the reducing agent cleavage reagent comprises: from 0.2 mol/L to 2 mol/L Tris-Base, from 0.2 mol/L to 2 mol/L sodium chloride, from 10 mmol/L to 100 mmol/L GSH, and from 0.01% to 0.1% Tween20, pH 8.0 to 10. For example, the reducing agent cleavage reagent is: 1 mol/L Tris-Base, 0.5 mol/L sodium chloride, 50 mmol/L GSH, and 0.05% Tween20, pH 9.0.

Thereafter, the extension products are washed to remove the labeling moiety that is cleaved, and the cleavage reagent.

Step 5 involves exciting the reaction products and collecting fluorescent signals therefrom to obtain a second image.

The methods of exciting the reaction products and collecting fluorescent signals therefrom in this step are the same as in step 3. In some examples, the fluorescent group of the reversible terminators added in step 2 includes a first fluorescent group and a second fluorescent group, and the excitation wavelength of the first fluorescent group differs from the excitation wavelength of the second fluorescent group by at least 50 nm, so as to increase the difference between the optical signal generated by the first fluorescent group and the optical signal generated by the second fluorescent group. The greater the difference between the excitation wavelength of the first fluorescent group and the excitation wavelength of the second fluorescent group, the more obvious the difference between the optical signal generated by the first fluorescent group and the optical signal generated by the second fluorescent group, and the easier the two fluorescent groups are to be identified.

In this step, an exposure treatment is performed after the fluorescent groups are excited to generate fluorescent signals. In some examples, the time of the exposure treatment is from 100 ms to 400 ms. By controlling the exposure time, the difference in the intensity of the optical signals generated by the fluorescent groups can be increased to a certain extent, thereby facilitating the identification of the type of the reversible terminator introduced at each site and improving the accuracy of base identification. For example, the time of the exposure treatment is 100 ms, 120 ms, 150 ms, 180 ms, 200 ms, 220 ms, 250 ms, 280 ms, 300 ms, 320 ms, 350 ms, 380 ms, 400 ms, etc. It should be understood that the time of the exposure treatment may be adjusted according to the type of the fluorescent groups so as to achieve the purpose of increasing the difference in the intensity of the optical signals generated by the fluorescent groups. In one example, the first fluorescent group is a red light-excitable fluorescent group, and the exposure time of the red light-excitable group is from 100 ms to 400 ms, such as 100 ms, 120 ms, 150 ms, 180 ms, 200 ms, 220 ms, 250 ms, 280 ms, 300 ms, 320 ms, 350 ms, 380 ms, or 400 ms; the second fluorescent group is a green light-excitable fluorescent group, and the exposure time of the green light-excitable group is from 50 ms to 100 ms, such as 50 ms, 60 ms, 70 ms, 80 ms, 90 ms, or 100 ms.

After the fluorescent groups are excited to generate optical signals, the reaction products are imaged to obtain a second image. The second image comprises a fifth signal, a sixth signal, a seventh signal, and an eighth signal from the first reversible terminator, the second reversible terminator, the third reversible terminator, and the fourth reversible terminator, respectively. In the examples of the present application, the fifth signal, the sixth signal, the seventh signal, and the eighth signal are each independently one of a first bright spot, a second bright spot, a third bright spot, and no bright spot, wherein the no bright spot indicates absence of a fluorescent signal, the first bright spot indicates a first fluorescent color feature, the second bright spot indicates a second fluorescent color feature, and the third bright spot indicates both the first fluorescent color feature and the second fluorescent color feature. It can be understood that the fluorescence color feature refers to a feature that can represent the fluorescence color, including the fluorescence color itself, and other indexes that can represent the fluorescence color. In the examples where the fluorescent group in the four types of reversible terminator comprises the first fluorescent group and/or the second fluorescent group, the first fluorescent color feature corresponds to the color of the fluorescence generated by the first fluorescent group when excited, and the second fluorescent color feature corresponds to the color of the fluorescence generated by the second fluorescent group when excited.

Hence, by virtue of the fluorescent signals provided by the first image in step 3, a subset of the four types of reversible terminator can be distinguished from the other subset of reversible terminators. Further, by introducing, via the reaction(s) of step 4, a difference between the reversible terminators that cannot be distinguished in step 3, and exhibiting the difference in the second image of step 5, the four types of reversible terminator are identified. Therefore, in the examples of the present application, the structures of the reversible terminators are designed such that the fluorescent signals generated by the four types of reversible terminator in the first image and the second image simultaneously satisfy:
a) the first signal and the fifth signal are the same,
b) the fourth signal and the eighth signal are the same,
c) the first signal and the fourth signal are different,
d) the second signal and the sixth signal are different,
e) the third signal and the seventh signal are different,
f) the sixth signal and the seventh signal are different and/or the second signal and the third signal are different,
so as to identify the type of the reversible terminators bound to the nucleic acid template-primer complexes.

According to different detection scenarios, the signal situations may be classified into at least one or more of the following:
the first signal and the fifth signal are both selected from one of the first bright spot or the second bright spot, and the fourth signal and the eighth signal are both selected from one of the first bright spot or the second bright spot; and the first signal, the second signal, the third signal, the sixth signal, and the seventh signal satisfy one of the following conditions 1) to 4):
1) the second signal is selected from the first bright spot or the second bright spot, the third signal is selected from the first bright spot or the second bright spot, the sixth signal is no bright spot, and the seventh signal is the third bright spot or no bright spot (the scenario shown in Table 2 or 5);
2) the second signal is selected from the first bright spot or the second bright spot, the third signal is no bright spot, the sixth signal is no bright spot, and the seventh signal is one of the first bright spot or the second bright spot; specifically, see Table 1, 3 or 4;
3) the second signal is no bright spot, the third signal is selected from the first bright spot or the second bright spot, the sixth signal is one of the first bright spot or the second bright spot, and the seventh signal is the third bright spot (the scenario shown in Table 6, 7 or 8);
4) the second signal is no bright spot, the third signal is no bright spot, the sixth signal is one of the first bright spot or the second bright spot, and the seventh signal is the third bright spot (the scenarios shown in Tables 9 and 10).

In the examples of the present application, for the scenarios in which identification of the four types of reversible terminator is achieved via two cleavage reactions (the first reaction and the third reaction) and the binding reaction (the second reaction), reference can be made to Table 1 to Table 5; for the scenarios in which identification of the four types of reversible terminator is achieved via one cleavage reaction (the third reaction) and the binding reaction (the second reaction), reference can be made to Table 6 to Table 10.

In one embodiment, the first signal, the second signal, the third signal, the fourth signal, the fifth signal, the sixth signal, the seventh signal, and the eighth signal satisfy the following conditions a) to e) at the same time, and in f), the sixth signal is different from the seventh signal, and the second signal is different from the third signal, as the scenarios shown in Table 1 to Table 8.

In another embodiment, the first signal, the second signal, the third signal, the fourth signal, the fifth signal, the sixth signal, the seventh signal, and the eighth signal satisfy the following conditions a) to e) at the same time, and in f), the sixth signal is different from the seventh signal, the scenarios shown in Table 1 to Table 10 being comprised within this range.

In some examples of the present application, when the structure of the four types of reversible terminator comprises the second cleavable group, since the second cleavable group, together with the labeling moiety such as the fluorescent group, is cleaved from the reversible terminators, and the first cleavable group and the labeling moiety such as the fluorescent group on the reversible terminators where the first cleavable group resides are retained. Therefore, when performing image acquisition, no fluorescent signal is collected from the reversible terminators having the second cleavable group, and a fluorescent signal can be collected from the reversible terminators having the first cleavable group. In the second image thus obtained, the reversible terminators comprising the second cleavable group no longer exhibit a fluorescent signal; by contrast, the reversible terminators comprising the first cleavable group either retain the same fluorescent signal as in the first image, or generate a fluorescent signal different from that in the first image by means of the labeling substance introduced via the second reaction.

Since the fluorescent groups of the reversible terminators having the same cleavable group have different fluorescent colors, or the fluorescent groups of the reversible terminators having different cleavable groups have the same fluorescent color, the reversible terminators having the same fluorescent color can be further distinguished by means of the cleavage in this step. Exemplarily, see Tables 1 to 10.

In some other examples of the present application, the structures of the four types of reversible terminator all have a single cleavable group, and the single cleavable group is any choice of the first cleavable group.

In step 7, the reaction products are subjected to the third reaction.

In this step, the third reaction is a reaction in which the first cleavable group or the second cleavable group in the reversible terminator structure is cleaved. By cleaving the cleavable group in the reversible terminator structure in this step, and the incorporated labeling moiety in the reversible terminator is removed. In an implementation, when step 4 includes the first reaction, that is, a reaction in which the second cleavable group is cleaved, and includes two cleavage reactions (the two cleavages include the first reaction and the third reaction, the first reaction being to cleave the second cleavable group, and the third reaction being a reaction in which the first cleavable group is cleaved), after the second cleavable group is cleaved in the first cleavage reaction, at least a subset of the four types of reversible terminator (hereinafter referred to as a first subset) can be distinguished from another subset of the reversible terminators (hereinafter referred to as a second subset), and in the process of cleaving the first cleavable group in the second cleavage reaction, the first subset of the four types of reversible terminator can be distinguished from each other, and the reversible terminators in the second subset can also be distinguished from each other. Hence, finally, the signals of the four types of reversible terminator with two-color fluorescence are distinguished one by one. This is specifically Scheme 1 of the two-color sequencing method of the examples of the present application. The exemplary signals and detection steps of Scheme 1 are as shown in Table 1 to Table 5.

When there is one cleavage reaction (the one cleavage includes the third reaction, the third reaction being a reaction in which the first cleavable group or the second cleavable group is cleaved; which cleavable group is specifically cleaved is determined according to the structure of the reversible terminators); signal information that enables distinguishment is generated following the first image, the binding reaction and the second image so that the four types of reversible terminator are distinguished, and the cleavable group is cleaved following the third reaction so that the next cycle of reaction can be performed normally. This is specifically Scheme 2 of the two-color sequencing method of the examples of the present application. The exemplary signals and detection steps of Scheme 2 are as shown in Table 6 to Table 10.

The conditions for the third reaction may vary depending on the type of the first cleavable group. In some examples, when the first cleavable group is disulfide bond, the reducing agent for cleaving the first cleavable group includes, but is not limited to, any one or more of DTT, glutathione, and/or β-mercaptoethanol.

In some examples, the concentration of the reducing agent is from 10 mmol/L to 100 mmol/L, and specifically, the concentration of the organic phosphine may be 10 mmol/L, 15 mmol/L, 20 mmol/L, 25 mmol/L, 30 mmol/L, 35 mmol/L, 40 mmol/L, 45 mmol/L, 50 mmol/L, 55 mmol/L, 60 mmol/L, 65 mmol/L, 70 mmol/L, 75 mmol/L, 80 mmol/L, 85 mmol/L, 90 mmol/L, 95 mmol/L, 100 mmol/L, etc.

In one example, glutathione is used as the reducing agent to cleave the first cleavable group, and the use concentration of glutathione is from 10 mmol/L to 100 mmol/L.

Thereafter, the extension products are washed to remove the fluorescent group that is cleaved, and the cleavage reagent.

In some examples, the first cleavable group is selected from at least one of —N3-, —CH₂F, —C(O)NHBu, —CHF₂, —C(O)OH, —C₆ and/or —ArC₆, or disulfide bond. Exemplarily, the first cleavable group is —N3-, and in this instance, the first cleavable group in the reversible terminators can be cleaved by adding an organophosphorus cleavage reagent. In some examples, the concentration of the organic phosphine is from 5 mmol/L to 20 mmol/L, and exemplarily, the concentration of the organic phosphine may be 5 mmol/L, 6 mmol/L, 7 mmol/L, 8 mmol/L, 9 mmol/L, 10 mmol/L, 11 mmol/L, 12 mmol/L, 13 mmol/L, 14 mmol/L, 15 mmol/L, 16 mmol/L, 17 mmol/L, 18 mmol/L, 19 mmol/L, 20 mmol/L, etc.

Exemplarily, the organophosphorus cleavage reagent for cleaving the first cleavable group includes, but is not limited to, any one or more of tris(3-hydroxypropyl)phosphine, tris(2-carboxyethyl)phosphine, trimethylolphosphine, triphenylphosphine, or tris(2-hydroxyethyl)phosphine. In one example, tris(3-hydroxypropyl)phosphine is used as the cleavage reagent to cleave the second cleavable group. The use concentration of tris(3-hydroxypropyl)phosphine is from 5 mmol/L to 20 mmol/L, and exemplarily, the concentration of the organic phosphine may be 5 mmol/L, 6 mmol/L, 7 mmol/L, 8 mmol/L, 9 mmol/L, 10 mmol/L, 11 mmol/L, 12 mmol/L, 13 mmol/L, 14 mmol/L, 15 mmol/L, 16 mmol/L, 17 mmol/L, 18 mmol/L, 19 mmol/L, 20 mmol/L, etc.

In some examples, the organophosphorus cleavage reagent comprises from 0.2 mol/L to 2 mol/L Tris-Base, from 0.2 mol/L to 2 mol/L sodium chloride, from 10 mmol/L to 100 mmol/L THPP, and from 0.01% to 0.1% Tween20, pH 8 to 10. Exemplarily, the tris(3-hydroxypropyl)phosphine cleavage reagent is 1 mol/L Tris-Base, 0.5 mol/L sodium chloride, 10 mmol/L THPP, and 0.05% Tween20, pH 9.0.

In some examples, when the structure of reversible terminator comprises disulfide bond as the only cleavable group, further comprised is performing blocking treatment on the cleavage site after any one of the above step 3 to step 7, or between these steps.

In the examples of the present application, step 2 to step 7 can be repeated once or more times, and each repetition will accomplish binding of one of the four types of reversible terminator onto the template-primer complex to complete base extension once and identify the base type. Thus, following multiple repetitions, two-color sequencing is accomplished and the base sequence of the template strain is obtained.

A two-color sequencing kit is provided, at least comprising four types of reversible terminator and a first cleavage reagent,
The four types of reversible terminator each independently comprise: a nucleotide or derivative thereof, a labeling moiety, and a first cleavable group linking the nucleotide or the derivative thereof and the labeling moiety, wherein the labeling moiety is selected from a fluorescent group and/or a specific binding label, at least two of the four modified nucleotides carry a first fluorescent group and a second fluorescent group that are optically distinguishable, respectively, one or two of the four modified nucleotides carry a specific binding label, the specific binding label being capable of specifically binding to a corresponding partner, and the first cleavage reagent is used to cleave the first cleavable group.

As a possible implementation of the two-color sequencing kit of the present application, the first cleavage reagent is an organophosphorus cleavage reagent.

As a possible implementation of the two-color sequencing kit of the present application, in the organophosphorus cleavage reagent, the organophosphorus is selected from any one or more of tris(3-hydroxypropyl)phosphine, tris(2-carboxyethyl)phosphine, trimethylolphosphine, triphenylphosphine, or tris(2-hydroxyethyl).

As a possible implementation of the two-color sequencing kit of the present application, in the organophosphorus cleavage reagent, the organophosphorus is tris(3-hydroxypropyl).

As a possible implementation of the two-color sequencing kit of the present application, the working concentration of the tris(3-hydroxypropyl)phosphine is from 10 mmol/L to 100 mmol/L.

As a possible implementation of the two-color sequencing kit of the present application, three of the four types of modified nucleotide carry the first fluorescent group and the second fluorescent group, which are optically distinguishable, respectively, and the other of the four types of modified nucleotide carries the specific binding label, and one of the two types of modified nucleotide carrying the same fluorescent group comprises a second cleavable group, the second cleavable group being so located as to link the first cleavable group and the labeling moiety, and the second cleavable group having a higher cleavability than the first cleavable group, and
the kit further comprises a second cleavage reagent for cleaving the second cleavable group.

As a possible implementation of the two-color sequencing kit of the present application, the second cleavage reagent is a reducing agent.

As a possible implementation of the two-color sequencing kit of the present application, in the reducing agent, the reducing agent is selected from DTT, glutathione, and/or β-SS-ethanol.

As a possible implementation of the two-color sequencing kit of the present application, the reducing agent is glutathione.

As a possible implementation of the two-color sequencing kit of the present application, the working concentration of glutathione is from 10 mmol/L to 100 mmol/L.

On the basis of any one of the above embodiments, as a possible implementation of the two-color sequencing kit of the present application, the structures of the four types of reversible terminator satisfy one or more of the following combinations (1)-(4):
(1) the structure of the first reversible terminator is: first nucleotide or derivative thereof-first cleavable group-first fluorescent group,
the structure of the second reversible terminator is: second nucleotide or derivative thereof-first cleavable group-specific binding label,
the structure of the third reversible terminator is: third nucleotide or derivative thereof-first cleavable group-specific binding label, and
the structure of the second reversible terminator is: fourth nucleotide or derivative thereof-first cleavable group-second fluorescent group;
(2) the structure of the first reversible terminator is: first nucleotide or derivative thereof-first cleavable group-first fluorescent group,
the structure of the second reversible terminator is: second nucleotide or derivative thereof-first cleavable group-specific binding label,
the structure of the third reversible terminator is: third nucleotide or derivative thereof-first cleavable group-specific binding label, and
the structure of the second reversible terminator is: fourth nucleotide or derivative thereof-first cleavable group-second fluorescent group-specific binding label;
(3) the structure of the first reversible terminator is: first nucleotide or derivative thereof-first cleavable group-first fluorescent group,
the structure of the second reversible terminator is: second nucleotide or derivative thereof-first cleavable group-specific binding label,
the structure of the third reversible terminator is: third nucleotide or derivative thereof-first cleavable group-second fluorescent group, and
the structure of the second reversible terminator is: fourth nucleotide or derivative thereof-first cleavable group-second cleavable group-first fluorescent group-specific binding label;
(4) the structure of the first reversible terminator is: first nucleotide or derivative thereof-first cleavable group-first fluorescent group,
the structure of the second reversible terminator is: second nucleotide or derivative thereof-first cleavable group-second fluorescent group-specific binding label,
the structure of the third reversible terminator is: third nucleotide or derivative thereof-first cleavable group-second fluorescent group, and
the structure of the second reversible terminator is: fourth nucleotide or derivative thereof-first cleavable group-second cleavable group-first fluorescent group;
Wherein in each combination, the first nucleotide or derivative thereof, the second nucleotide or derivative thereof, the third nucleotide or derivative thereof, and the fourth nucleotide or derivative thereof are selected from one of adenine or derivative thereof, thymine or derivative thereof, cytosine or derivative thereof, or guanine or derivative thereof, and the first nucleotide or derivative thereof, the second nucleotide or derivative thereof, the third nucleotide or derivative thereof, and the fourth nucleotide or derivative thereof are different from each other.

On the basis of any one of the above embodiments, as a possible implementation of the two-color sequencing kit of the present application, the first cleavable group is selected from groups comprising at least one of —N3-, —CH₂F, —C(O)NHBu, —CHF₂, —C(O)OH, —C6-, —ArC6-, and/or disulfide bond.

As a possible implementation of the two-color sequencing kit of the present application, the first cleavable group is selected from at least one of —N₃—, —CH₂F, —C(O)NHBu, —CHF₂, —C(O)OH, —C₆—ArC₆, and/or disulfide.

As a possible implementation of the two-color sequencing kit of the present application, the four types of reversible terminator all comprise only the first cleavable group, and the first cleavable group is —N₃— or disulfide bond.

As a possible implementation of the two-color sequencing kit of the present application, the four types of reversible terminator each independently comprise the first cleavable group and/or the second cleavable group, the first cleavable group is —N₃—, and the second cleavable group is selected from groups comprising disulfide bond. As a possible implementation of the two-color sequencing kit of the present application, the second cleavable group is disulfide bond.

On the basis of any one of the above embodiments, as a possible implementation of the two-color sequencing kit of the present application, the first fluorescent group and the second fluorescent group are each independently selected from one of a red fluorescent group, a green fluorescent group, and a blue fluorescent group.

As a possible implementation of the two-color sequencing kit of the present application, the first fluorescent group and the second fluorescent group are each independently selected from: one or combinations of more of Atto532, Atto647N, Atto390, Atto Rho6G, Alexa Fluor405, Alexa Fluor532, GM656, Alexa Fluor647, CY5, IF700, ROX, CY3, CY5 rhodamine dye, cyanine dye, coumarin dye, or phycoerythrin.

As a possible implementation of the two-color sequencing kit of the present application, a combination of the first fluorescent signal and the second fluorescent signal is a combination formed of red and green; or a combination of the first fluorescent signal and the second fluorescent signal is a combination formed of red and blue.

As a possible implementation of the two-color sequencing kit of the present application, the fluorescent group whose fluorescent color is green includes Atto532, Alexa Fluor532, Atto Rho6G, or CY3;
the fluorescent group whose fluorescent color is red includes: Atto647N, CY5, GM656, or Alexa Fluor647;
the fluorescent group whose fluorescent color is blue includes: Atto390, Alexa Fluor405, or coumarin.

On the basis of any one of the above embodiments, as a possible implementation of the two-color sequencing kit of the present application, the specific binding label is selected from at least one of biotin, digoxin, hapten, or functional derivatives thereof.

On the basis of any one of the above embodiments, as a possible implementation of the two-color sequencing kit of the present application, the reversible terminators include at least one group shown in the following Combinations 1 to 25:
Combination 1: adenine nucleotide or derivative thereof-N3-Atto647N, guanine nucleotide or derivative thereof-N3-SS-Atto647N, thymine nucleotide or derivative thereof-N3-Biotin, and cytosine nucleotide or derivative thereof-N3-Atto532;
Combination 2: adenine nucleotide or derivative thereof-N3-CY5, guanine nucleotide or derivative thereof-N3-SS-Atto647N, thymine nucleotide or derivative thereof-N3-Biotin, and cytosine nucleotide or derivative thereof-N3-Atto532;
Combination 3: adenine nucleotide or derivative thereof-N3-CY5, guanine nucleotide or derivative thereof-N3-SS-CY5, thymine nucleotide or derivative thereof-N3-Biotin, and cytosine nucleotide or derivative thereof-N3-Atto532;
Combination 4: adenine nucleotide or derivative thereof-N3-Atto647N, guanine nucleotide or derivative thereof-N3-SS-CY5, thymine nucleotide or derivative thereof-N3-Biotin, and cytosine nucleotide or derivative thereof-N3-Atto532;
Combination 5: adenine nucleotide or derivative thereof-N3-Atto647N, guanine nucleotide or derivative thereof-N3-SS-Atto647N, thymine nucleotide or derivative thereof-N3-Atto532-Biotin, and cytosine nucleotide or derivative thereof-N3-Atto532;
Combination 6: adenine nucleotide or derivative thereof-N3-Atto647N, guanine nucleotide or derivative thereof-N3-SS-CY5, cytosine nucleotide or derivative thereof-N3-Atto532-Biotin, and cytosine nucleotide or derivative thereof-N3-Atto532;
Combination 7: adenine nucleotide or derivative thereof-N3-CY5, guanine nucleotide or derivative thereof-N3-SS-Atto647N, thymine nucleotide or derivative thereof-N3-Atto532-Biotin, and cytosine nucleotide or derivative thereof-N3-Atto532;
Combination 8: adenine nucleotide or derivative thereof-N3-CY5, guanine nucleotide or derivative thereof-N3-SS-CY5, thymine nucleotide or derivative thereof-N3-Atto532-Biotin, and cytosine nucleotide or derivative thereof-N3-Atto532;
Combination 9: adenine nucleotide or derivative thereof-N3-Atto647N, guanine nucleotide or derivative thereof-N3-SS-Atto532, thymine nucleotide or derivative thereof-N3-Biotin, and cytosine nucleotide or derivative thereof-N3-Atto532;
Combination 10: adenine nucleotide or derivative thereof-N3-CY5, guanine nucleotide or derivative thereof-N3-SS-Atto532, thymine nucleotide or derivative thereof-N3-Biotin, and cytosine nucleotide or derivative thereof-N3-Atto532;
Combination 11: adenine nucleotide or derivative thereof-N3-Atto532, guanine nucleotide or derivative thereof-N3-SS-Atto532, cytosine nucleotide or derivative thereof-N3-Biotin, and cytosine nucleotide or derivative thereof-N3-Atto647N;
Combination 12: adenine nucleotide or derivative thereof-N3-Atto532, guanine nucleotide or derivative thereof-N3-SS-Atto532, thymine nucleotide or derivative thereof-N3-Biotin, and cytosine nucleotide or derivative thereof-N3-CY5;
Combination 13: adenine nucleotide or derivative thereof-N3-Atto532, guanine nucleotide or derivative thereof-N3-SS-Atto532, cytosine nucleotide or derivative thereof-N3-Atto647N-Biotin, and cytosine nucleotide or derivative thereof-N3-Atto647N;
Combination 14: adenine nucleotide or derivative thereof-N3-Atto532, guanine nucleotide or derivative thereof-N3-SS-Atto532, thymine cytosine nucleotide or derivative thereof-N3-CY5-Biotin, and cytosine nucleotide or derivative thereof-N3-CY5;
Combination 15: adenine nucleotide or derivative thereof-N3-Atto647N, guanine nucleotide or derivative thereof-N3-Biotin, thymine nucleotide or derivative thereof-N3-Atto532-Biotin, and cytosine nucleotide or derivative thereof-N3-Atto532;
Combination 16: adenine nucleotide or derivative thereof-N3-CY5, guanine nucleotide or derivative thereof-N3-Biotin, thymine nucleotide or derivative thereof-N3-Atto532-Biotin, and cytosine nucleotide or derivative thereof-N3-Atto532;
Combination 17: adenine nucleotide or derivative thereof-N3-Atto647N, guanine nucleotide or derivative thereof-N3-Biotin, thymine cytosine nucleotide or derivative thereof-N3-Atto647N-Biotin, and cytosine nucleotide or derivative thereof-N3-Atto532;
Combination 18: adenine nucleotide or derivative thereof-N3-CY5, guanine nucleotide or derivative thereof-N3-Biotin, thymine nucleotide or derivative thereof-N3-Atto647N-Biotin, and cytosine nucleotide or derivative thereof-N3-Atto532;
Combination 19: adenine nucleotide or derivative thereof-N3-Atto647N, guanine nucleotide or derivative thereof-N3-Biotin, thymine nucleotide or derivative thereof-N3-CY5-Biotin, and cytosine nucleotide or derivative thereof-N3-Atto532;
Combination 20: adenine nucleotide or derivative thereof-N3-Atto532, guanine nucleotide or derivative thereof-N3-Biotin, thymine cytosine nucleotide or derivative thereof-N3-Atto532-Biotin, and cytosine nucleotide or derivative thereof-N3-Atto647N;
Combination 21: adenine nucleotide or derivative thereof-N3-Atto532, guanine nucleotide or derivative thereof-N3-Biotin, thymine cytosine nucleotide or derivative thereof-N3-Atto532-Biotin, and cytosine nucleotide or derivative thereof-N3-CY5;
Combination 22: adenine nucleotide or derivative thereof-N3-Atto647N, guanine nucleotide or derivative thereof-N3-Biotin, thymine cytosine nucleotide or derivative thereof-N3-Biotin, and cytosine nucleotide or derivative thereof-N3-Atto532;
Combination 23: adenine nucleotide or derivative thereof-N3-CY5, guanine nucleotide or derivative thereof-N3-Biotin, thymine nucleotide or derivative thereof-N3-Biotin, and cytosine nucleotide or derivative thereof-N3-Atto532;
Combination 24: adenine nucleotide or derivative thereof-N3-Atto532, guanine nucleotide or derivative thereof-N3-Biotin, thymine cytosine nucleotide or derivative thereof-N3-Biotin, and cytosine nucleotide or derivative thereof-N3-Atto647N;
Combination 25: adenine nucleotide or derivative thereof-N3-Atto532, guanine nucleotide or derivative thereof-N3-Biotin, thymine cytosine nucleotide or derivative thereof-N3-Biotin, and cytosine nucleotide or derivative thereof-N3-CY5.

As a possible implementation of the two-color sequencing kit of the present application, in Combinations 1 to 14, the cleavable group —N3- in any one of the reversible terminators not having the specific binding label is replaced with —N3-SS—, and the cleavable group —N3-SS— in any one of the reversible terminators not having the specific binding label is replaced with —N3-; and/or
in Combinations 1 to 14, —N3-SS— is replaced with —SS—.

As a possible implementation of the two-color sequencing kit of the present application, in Combinations 15 to 25, CY5 and/or Atto647N are/is replaced with GM656 or Alexa Fluor647; and/or Atto532 and/or Alexa Fluor532 are/is replaced with Atto Rho6G or CY3; or
in Combinations 15 to 25, CY5 and/or Atto647N are/is replaced with one of the blue light dyes.

On the basis of any one of the above embodiments, as a possible implementation of the two-color sequencing kit of the present application, the working concentration of the adenine nucleotide or derivative thereof is from 10 nmol/L to 700 nmol/L, the working concentration of the thymine nucleotide or derivative thereof is from 5 nmol/L to 60 nmol/L, the working concentration of the cytosine nucleotide or derivative thereof is from 10 nmol/L to 700 nmol/L, and the working concentration of the guanine nucleotide or derivative thereof is from 0.05 nmol/L to 5 nmol/L.

Explanations are provided below in conjunction with specific examples.

### Example 1 Adsorption Test for Two-Color Sequencing

This experiment was performed on a single-molecule sequencing platform GenoCare1600 from GeneMind. Due to the non-specific adsorption of nucleotides or analogs thereof on the surface of the solid-phase carrier in the single-molecule sequencing platform, the recognition of base signals during the sequencing process would be greatly interfered with, which would affect the sequencing quality. Therefore, an adsorption test was performed on the concentrations of nucleotides or analogs thereof so as to determine the appropriate concentrations of the four types of nucleotides and the polymerase during the base extension reactions.

The method of the adsorption test was as follows:
A surface-treated solid-phase carrier was provided. The solid-phase carrier comprised a plurality of sites, each site having the ability to immobilize a nucleic acid template-primer complex, but not being immobilized with a nucleic acid template-primer complex.

Nucleotide solutions of dA-N3-Atto647N, dT-N3-SS-Atto532, dC-N3-Alexa Fluor532, and dG-N3-SS-CY5 were prepared, respectively. 9°N enzyme was added to each nucleotide solution to form an extension reagent. The extension reagent containing dA-N3-Atto647N and 9°N enzyme was designated dA-N3-Atto647N extension reagent, the extension reagent containing dT-N3-SS-Atto532 and 9°N enzyme was designated dT-N3-SS-Atto532 extension reagent, the extension reagent containing dC-N3-Alexa Fluor532 and 9°N enzyme was designated dC-N3-Alexa Fluor532 extension reagent, and the extension reagent containing dG-N3-SS-CY5 and 9°N enzyme was designated dG-N3-SS-CY5 extension reagent. Different nucleotide concentrations and 9°N enzyme concentrations were set for each extension reagent.

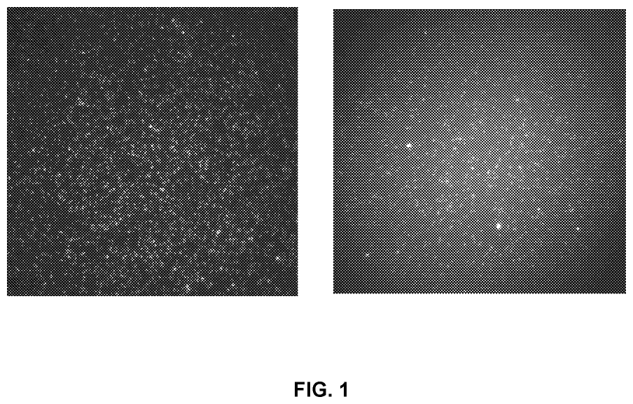

The surface of the solid-phase carrier was treated using excitation light whose excitation wavelength was consistent with that of the fluorescent group carried by the extension reagent to be added, and the solid-phase carrier after the excitation treatment was imaged to obtain a first image. Specifically, each extension reagent was added to the surface of the solid-phase carrier, respectively, the fluorescent group on each extension reagent was excited (excitation wavelength was 532 nm or 640 nm) to generate an optical signal, and the solid-phase carrier was imaged to obtain an image. The result is as shown in FIG. 1.

The image was subjected to data analysis, and the average number of fluorescence signal points (number of adsorption points) in the image was counted. According to the statistical results, the concentrations of the nucleotide solutions and the polymerase in the subsequent examples were set as follows: dA-N3-Atto647N: from 10 nmol/L to 500 nmol/L, dC-N3-Alexa Fluor532: from 100 nmol/L to 700 nmol/L, dT-N3-SS-Atto532: from 20 nmol/L to 80 nmol/L, dG-N3-SS-CY5: from 0.1 nmol/L to 5 nmol/L, 9°N concentration: from 0.01 mg/L to 0.1 mg/L.

### Example 2 Test of the Efficiency of Cleavage of Disulfide Bond by Glutathione (GSH)

The method was as follows:

Step 1: Preparation of an extension reagent: ddG-SS-Atto647N was selected for testing. 9°N enzyme was added to the ddG-SS-Atto647N solution to prepare ddG-SS-Atto647N extension reagent; In the extension reagent, the concentration of 9°N enzyme was 0.02 mg/ml; the concentration of ddG-SS-Atto647N was 5 nmol/L.

Step 2: Under the condition of a temperature of 60 °C, the synthesis reagent (extension reagent) was introduced onto the surface of a solid-phase carrier to allow reaction for 2 min.

After completion of the reaction, the surface of the solid-phase carrier was washed.

Step 3: The surface of the solid-phase carrier was irradiated using excitation light at 640 nm to excite the fluorescent group on the extension reagent, so that the fluorescent group generated an optical signal. Then fluorescent signals from the solid-phase carrier were collected to obtain a first image.

Step 4: After the first image was obtained, the surface of the solid-phase carrier was treated with a GSH cleavage reagent to cleave —SS—, the conditions for the treatment being reaction at 37 °C for 2 min.

The GSH cleavage reagent comprised: 1 mol/L Tris-Base, 0.5 mol/L sodium chloride, 0.05% (v/v) Tween20 and GSH, pH 9.0. Four parallel groups of test with GSH concentrations of 10 mmol/L, 20 mmol/L, 30 mmol/L, and 50 mmol/L, respectively, were set up.

Step 5: The surface of the solid-phase carrier was irradiated with excitation light from a 532 nm laser and a 640 nm laser respectively, and optical signals were collected to obtain a second image.

Step 6. The fluorescence signals of the two images were counted, and the efficiency of cleavage was analyzed.

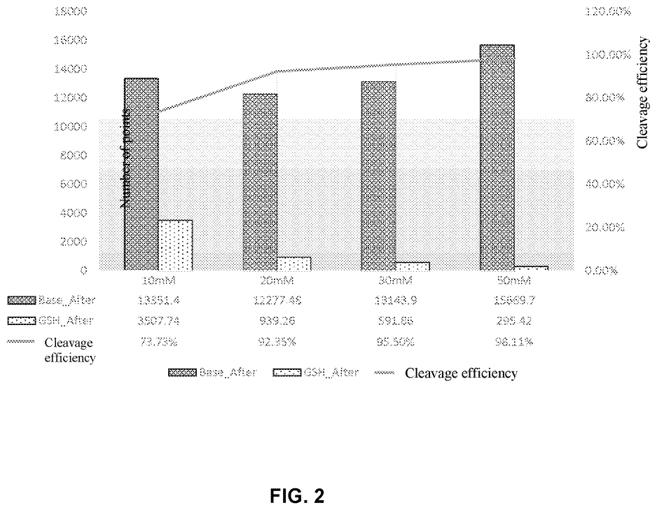

The cleavage percentage of —SS—=(the number of optical signal points before cleavage−the number of optical signal points after cleavage)/the number of optical signal points before cleavage×100%; alternatively, the residual percentage of disulfide bond on the surface of the solid-phase carrier: the residual percentage of disulfide bond=the number of optical signal points after cleavage/the number of optical signal points before cleavage×100%). The results are as shown in FIG. 2. The GSH cleavage reagent had a good cleavage effect on —SS—, and the cleavage effect was better in the concentration range of from 20 mmol/L to 50 mmol/L.

### Example 3 Test of the Efficiency of Cleavage of Azidomethoxy Group by THPP

The method was as follows:

Step 1: Preparation of extension reagents: dA-N3-Atto647N and dC-N3-Atto532 were selected for the test. Nucleotide solutions of dA-N3-Atto647N and dC-N3-Atto532 were prepared, respectively. 9°N enzyme was added to each of the nucleotide solutions to form an extension reagent. The extension reagent containing dA-N3-Atto647N and 9°N enzyme was designated dA-N3-Atto647N extension reagent, and the extension reagent containing dC-N3-Atto532 and 9°N enzyme was designated dC-N3-Atto532 extension reagent. In the extension reagents, the concentration of 9°N enzyme was set at 0.02 mg/ml; the concentrations of dA-N3-Atto647N and dC-N3-Atto532 were 50 nmol/L and 500 nmol/L, respectively.

Step 2: Under the condition of a temperature of 37 °C, the synthesis reagent was introduced onto the surface of a solid-phase carrier to allow reaction for 2 min. After completion of the reaction, the surface of the solid-phase carrier was washed.

Step 3: The surface of the solid-phase carrier was irradiated using excitation light at 532 nm and 640 nm, respectively, to excite the fluorescent group on the extension reagent to generate an optical signal. Fluorescent signals from the solid-phase carrier were collected to obtain a first image.

Step 4: The THPP cleavage reagent was used to perform a reaction at 37 °C for 2 min on the surface of the solid-phase carrier to cleave the azidomethoxy group.

The THPP cleavage reagent comprised: 1 mol/L Tris-Base, 0.5 mol/L sodium chloride, and 0.05% Tween20 and THPP by volume fraction, pH 9.0; Parallel experiments with THPP concentrations of 10 mmol/L, 25 mmol/L, 50 mmol/L, and 100 mmol/L, respectively, were set up.

Step 5: The surface of the solid-phase carrier was irradiated with excitation light from 532 nm and 640 nm lasers respectively, and optical signals were collected to obtain a second image.

Step 6. The fluorescence signals of the two images were counted, and the efficiency of cleavage was analyzed.

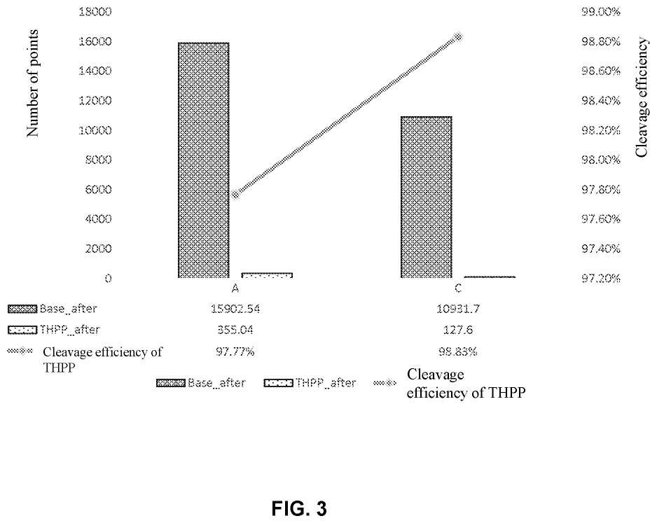

The cleavage percentage of azidomethoxy group=(the number of optical signal points before cleavage−the number of optical signal points after cleavage)/the number of optical signal points before cleavage×100%; alternatively, the residual percentage of azidomethoxy group=the number of optical signal points after cleavage/the number of optical signal points before cleavage×100%). The result showed that THPP had a good cleavage effect on azidomethoxy group. The cleavage effect when the concentration of THPP was 10 mmol/L is as shown in FIG. 3.

### Example 4 the Efficiency of Binding of SA-CY3 with dUTP-Biotin

SA-CY3 and dUTP-Biotin purchased from Sangon Biotech Co., Ltd. were used to verify the efficiency of binding of SA-CY3 with dU-Biotin on a single-molecule sequencing chip, the binding being a key step of two-color paired-end sequencing.

After reconstitution, the chip was hybridized with a single-stranded nucleic acid molecule having CY5 and having T base for the first cycle of reaction (designated as: D9-T-CY5). Synthesis reagents, comprising 10 nmol/L, 125 nmol/L, and 1000 nmol/L dU-Biotin, respectively, and 0.01 mg/ml 9°N, were prepared and were allowed to react under the condition of a temperature of 55 °C for 2 min. SA-CY3 was added at concentrations of 10 nmol/L, 50 nmol/L, and 125 nmol/L, respectively, and was allowed to react at 37 °C for 90 s. The composition of the buffer in the synthesis reagents and the reaction reagents containing SA-CY3 was: 50 mmol/L Tris-Base pH 8.8, 50 mmol/L NaCl, 10 mmol/L (NH₄)₂SO₄, and 3 mmol/L MgSO₄. At the same time, dT-Atto532 was used instead of dU-Biotin as a control group.

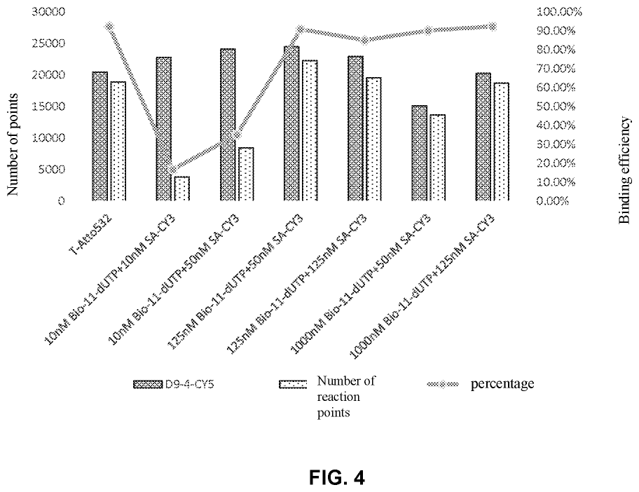

The surface of the chip was irradiated with excitation light from a 532 nm laser and a 640 nm laser, respectively, and optical signals were collected. The efficiency of binding was estimated by counting the number of adsorbed single molecules excited by the 532 nm laser and the number of adsorbed single molecules excited by the 640 nm laser, as shown in FIG. 4.

It can be seen from FIG. 4 that a concentration of dUTP-Biotin higher than 125 mmol/L would be beneficial to improving the efficiency of binding of Biotin with SA-CY3. In addition, a high concentration of SA-CY3 would be beneficial to improving the efficiency of binding. The efficiency of binding of 125 nM dUTP-Biotin with 50 nM SA-CY3 was about 90%, similar to the reaction efficiency of the two-color base dT-Atto532 as the control group.

### Example 5 Time of Binding of SA-CY3 with dUTP-Biotin

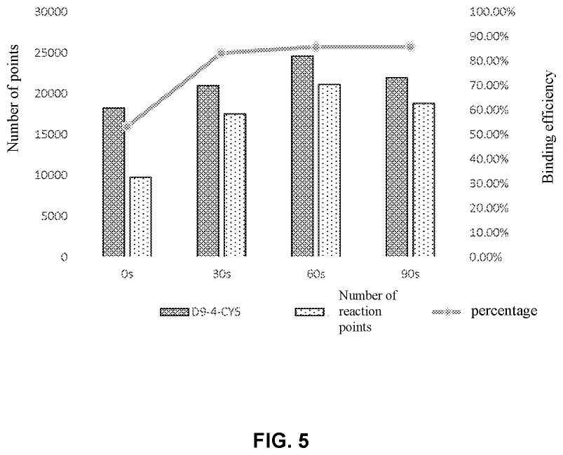

The method of Example 4 was followed, with the concentration of dU-Biotin being set at 125 nmol/L dUTP-Biotin and the concentration of SA-CY3 being set at 50 nmol/L. The binding time of SA-CY3 was set in a gradient of 0 s, 30 s, 60 s, and 90 s. The ratio of the number of SA-CY3 adsorbed single molecules to the number of D9-T-CY5 single molecule points was counted. The result is as shown in FIG. 5.

It can be seen from FIG. 5 that the efficiency of binding of SA-CY3 with dUTP-Biotin was relatively high when the time of binding was in the range of from 30 s to 90 s, and a further increase in the time of binding did not have an obvious effect in further improving the efficiency of binding. The verification of the time of binding confirmed that the time could be shortened to 30 s.

### Example 6 Test of Adsorption by SA-CY3

A chip was reconstituted and was not subjected to hybridization treatment. A first image was collect to determine background adsorption. Then the chip was hybridized with a single-stranded nucleic acid molecule having CY5 and having T base for the first cycle of reaction (designated as: D9-T-CY5). 125 nmol/L dUTP-Biotin was added for polymerization reaction, and then 50 nmol/L SA-CY3 was added for reaction at 37 °C was added for reaction for 90 s. A second image was collected, and adsorption by SA-CY3 was analyzed, as shown in Table 11.

TABLE 11
Adsorption by SA-CY3

| | Number of | Number of single |
|---|---|---|
| | background | molecules |
| Reaction condition | points | adsorbed by SA-CY3 |
| 125 nM Bio-11-dUTP + 50 nM | 171.72 | 291.03 |
| SA-CY3 | | |
| 1000 nM Bio-11-dUTP + 50 nM | | 293.15 |
| SA-CY3 | | |

It can be seen from Table 11 that the overall adsorption by SA-CY3 was low, and was within the standard range of 500.

### Example 7 Two-Color Sequencing Method

The following reagents were prepared:
Synthesis reagents: synthesis reagents of dA-N3-Atto647N (10 nmol/L), dT-N3-Biotin (30 nmol/L), dC-N3-Atto532 (500 nmol/L), and dG-N3-SS-Atto647N (1 nmol/L), and 0.01 mg/ml 9°N; the buffer in synthetic reagents was: 50 mmol Tris-Base pH 8.8, 50 mmol NaCl, 10 mmol (NH₄)₂SO₄, 3 mmol MgSO₄;
Cleavage reagent 1: 2 mol/L Tris-Base, 1 mol/L sodium chloride, 50 mmol/L GSH, and 0.05% Tween20 by volume, pH 9.0;
Cleavage reagent 2: 2 mol/L Tris-Base, 1 mol/L sodium chloride, 10 mmol/L THPP, and 0.05% Tween20, pH 9.0;
A two-color sequencing method comprised:
(1) after reconstituting a chip, hybridizing the chip with a gDNA library;
(2) adding the synthetic reagents onto the surface of the chip, irradiating the surface of the solid-phase carrier with excitation light from a 532 nm laser and a 640 nm laser for 80 m and 150 ms, respectively, to image the solid-phase carrier, and collecting a first image;
(3) adding the cleavage reagent 1 onto the surface of the chip to perform a cleavage reaction on —SS—;
(4) adding 50 nM SA-CY3 onto the surface of the solid-phase carrier to allow reacting (binding) for 30 s;
(5) irradiating the surface of the solid-phase carrier with excitation light from the 532 nm laser and the 640 nm laser for 80 ms and 150 ms, respectively, to image the solid-phase carrier, and collecting a second image;
(6) adding the cleavage reagent 2 onto the surface of the chip to perform a cleavage reaction on the azide group; and
(7) repeating the above steps (1) to (6) 50 times, that is, performing 50 cycles of sequencing. The average values of sequencing throughput, read length, and error ratio for the four lanes of the chip during the 50 cycles were calculated. The results are as shown in Table 12 below. The throughput included the number of Reads uniquely aligning with the genome in the central 1024×1024 pixel region of 400 fields of view, i.e., FOV. The error ratio (error ratio=mismatch ratio+deletion type error ratio+insertion type error ratio) was less than 9.5%, and the stability was good.

TABLE 12

| | Mismatch | Deletion type error | Insertion type error | |
|---|---|---|---|---|
| Lane | ratio | ratio | ratio | Error ratio |
| Lane 1 | 3.02% | 2.95% | 2.93% | 8.90% |
| Lane 2 | 2.93% | 3.25% | 2.71% | 8.88% |
| Lane 3 | 2.95% | 3.42% | 2.63% | 9.00% |
| Lane 4 | 3.18% | 3.53% | 2.78% | 9.49% |

The following are a series of sequentially numbered statements of the invention, not claims. (Claims follow on after these statements and are headed “CLAIMS”)
1. A two-color sequencing method, characterized in that the two-color sequencing method comprises the following steps:
step 1, providing four types of reversible terminator: a first reversible terminator, a second reversible terminator, a third reversible terminator, and a fourth reversible terminator, the four types of reversible terminator each independently comprising: a nucleotide or derivative thereof, a labeling moiety, and a first cleavable group and/or a second cleavable group linking the nucleotide or derivative thereof and the labeling moiety, the nucleotide or derivative thereof being selected from adenine or derivative thereof, thymine or derivative thereof, cytosine or derivative thereof, or guanine or derivative thereof,
step 2, allowing the reversible terminators to bind to and extend along nucleic acid template-primer complexes via polymerase chain reactions to obtain extension products;
step 3, exciting the extension products and collecting fluorescent signals therefrom to obtain a first image, the first image comprising a first signal, a second signal, a third signal, and a fourth signal from the first reversible terminator, the second reversible terminator, the third reversible terminator, and the fourth reversible terminator, respectively;
step 4, performing a first reaction and a second reaction on the extension products, or performing a second reaction on the extension products, to obtain reaction products;
step 5, exciting the reaction products and collecting fluorescent signals therefrom to obtain a second image, the second image comprising a fifth signal, a sixth signal, a seventh signal, and an eighth signal from the first reversible terminator, the second reversible terminator, the third reversible terminator, and the fourth reversible terminator, respectively;
step 6, determining the type of the reversible terminators bound to the nucleic acid template-primer complexes according to the optical signals of the first image and the second image;
step 7, performing a third reaction on the reaction products; and
step 8, repeating step 2 to step 7 one or more times to determine the sequence of the template nucleic acids;
wherein
the labeling moiety is selected from: a fluorescent group and/or a specific binding label, the specific binding label being configured to bind to a labeling substance, and the labeling substance comprising a ligand and a fluorescent group bound to the ligand;
the first reaction is a reaction in which the second cleavable group is cleaved;
the second reaction is a binding reaction between the ligand and the specific binding label;
the third reaction is a reaction in which the first cleavable group or the second cleavable group is cleaved;
when the reversible terminator comprises both the first cleavable group and the second cleavable group, the first cleavable group and the second cleavable group are different, and the second cleavable group has a higher cleavability than the first cleavable group;
the first signal, the second signal, the third signal, the fourth signal, the fifth signal, the sixth signal, the seventh signal, and the eighth signal are each independently one of a first bright spot, a second bright spot, a third bright spot, and no bright spot, wherein the first bright spot indicates a first fluorescent color feature, the second bright spot indicates a second fluorescent color feature, and the third bright spot indicates both the first fluorescent color feature and the second fluorescent color feature,
and the first signal, the second signal, the third signal, the fourth signal, the fifth signal, the sixth signal, the seventh signal, and the eighth signal satisfy the following conditions a) to f) at the same time:
a) the first signal and the fifth signal are the same,
b) the fourth signal and the eighth signal are the same,
c) the first signal and the fourth signal are different,
d) the second signal and the sixth signal are different,
e) the third signal and the seventh signal are different,
and f) the sixth signal and the seventh signal are different and/or the second signal and the third signal are different.
2. The two-color sequencing method according to claim 1, characterized in that,
optionally, the labeling moiety comprises the fluorescent group and the specific binding label, and the fluorescent group in the labeling moiety and the fluorescent group in the labeling substance are selected from the first fluorescent group and the second fluorescent group, which are distinguishable in color;
optionally, among the reversible terminators having the same cleavable group, there are at least two types of reversible terminator whose labeling moieties have different fluorescent colors;
optionally, the first cleavable group is selected from groups comprising at least one of —N₃—, —CH₂F, —C(O)NHBu, —CHF₂, —C(O)OH, —C6, —ArC6, and/or disulfide bond;
optionally, the first cleavable group is selected from at least one of —N₃—, —CH₂F, —C(O)NHBu, —CHF₂, —C(O)OH, —C₆—ArC₆, and/or disulfide bond;
optionally, the first cleavable group is —N3- or disulfide;
optionally, the second cleavable group is selected from groups comprising disulfide;
optionally, the second cleavable group is disulfide bond;
optionally, the fluorescent group is selected from the first fluorescent group and the second fluorescent group, which are distinguishable in color;
optionally, in the first signal to the eighth signal,
the first signal, the second signal, and the fifth signal are the same, the fourth signal and the eighth signal are the same, the first signal and the fourth signal are different, the second signal and the third signal are different, the second signal, the fourth signal, and the sixth signal are different from each other, and the third signal and the seventh signal are different from each other;
or, the first signal and the fifth signal are the same, the fourth signal and the eighth signal are the same, the first signal and the fourth signal are different, the second signal, the first signal, and the third signal are different from each other, and the third signal, the fourth signal, and the seventh signal are different from each other;
or, the first signal, the fifth signal, and the sixth signal are the same, the third signal, the fourth signal, and the eighth signal are the same, the first signal and the third signal are different, the second signal is different from the first signal and the third signal, respectively, and the seventh signal and the third signal are different;
or, the first signal and the fifth signal are the same, the fourth signal and the eighth signal are the same, the first signal and the fourth signal are different, the second signal and the third signal are the same, the second signal is different from the sixth signal and the seventh signal, respectively, and the seventh signal is different from the third signal and the sixth signal, respectively;
or, the first signal, the third signal, and the fifth signal are the same, the fourth signal and the eighth signal are the same, the first signal and the fourth signal are different, the second signal and the third signal are different, the second signal, the first signal, and the fourth signal are different from each other, and the third signal, the fourth signal, and the seventh signal are different from each other.
3. The two-color sequencing method according to claim 2, characterized in that,
the first signal and the fifth signal are both selected from one of the first bright spot or the second bright spot, the fourth signal and the eighth signal are both selected from one of the first bright spot or the second bright spot; and the first signal, the second signal, the third signal, the sixth signal, and the seventh signal satisfy one of the following conditions 1) to 4):
1) the second signal is selected from the first bright spot or the second bright spot, the third signal is selected from the first bright spot or the second bright spot, the sixth signal is the no bright spot, and the seventh signal is the third bright spot;
2) the second signal is selected from the first bright spot or the second bright spot, the third signal is the no bright spot, the sixth signal is the no bright spot, and the seventh signal is one of the first bright spot or the second bright spot;
3) the second signal is the no bright spot, the third signal is selected from the first bright spot or the second bright spot, the sixth signal is one of the first bright spot or the second bright spot, and the seventh signal is the third bright spot;
4) the second signal is no bright spot, the third signal is no bright spot, the sixth signal is one of the first bright spot or the second bright spot, and the seventh signal is the third bright spot.
4. The two-color sequencing method according to claim 3, characterized in that,
one or more of the four types of reversible terminator comprise(s) the first cleavable group, and one or more of the four types of reversible terminator comprise(s) the second cleavable group;
optionally, the first signal, the second signal, the third signal, the sixth signal, and the seventh signal satisfy condition 1) or condition 2), and the structures of the four types of reversible terminator satisfy the following:
the structure of the first reversible terminator is: first nucleotide or derivative thereof-first cleavable group-first fluorescent group,
the structure of the second reversible terminator is one of the following scenarios: second nucleotide or derivative thereof-first cleavable group-second cleavable group-first fluorescent group or second fluorescent group; or second nucleotide or derivative thereof-second cleavable group-first fluorescent group or second fluorescent group;
the structure of the third reversible terminator is one of the following scenarios: third nucleotide or derivative thereof-first cleavable group-specific binding label; or third nucleotide or derivative thereof-first cleavable group-first fluorescent group or second fluorescent group-specific binding label;
the structure of the fourth reversible terminator is: fourth nucleotide or derivative thereof
first cleavable group-second fluorescent group;
the second reversible terminator and the third reversible terminator do not comprise the specific binding label at the same time;
wherein, the first nucleotide or derivative thereof, the second nucleotide or derivative thereof, the third nucleotide or derivative thereof, and the fourth nucleotide or derivative thereof are selected from one of adenine or derivative thereof, thymine or derivative thereof, cytosine or derivative thereof, or guanine or derivative thereof, and the first nucleotide or derivative thereof, the second nucleotide or derivative thereof, the third nucleotide or derivative thereof, and the fourth nucleotide or derivative thereof are different from each other;
optionally, the first fluorescent group and the second fluorescent group are each independently selected from: one or combinations of more of Atto532, Atto647N, Atto390, Atto Rho6G, Alexa Fluor405, Alexa Fluor532, GM656, Alexa Fluor647, CY5, IF700, ROX, CY3, CY5 rhodamine dye, cyanine dye, coumarin dye, or phycoerythrin;
optionally, the fluorescent color of the first fluorescent group is red, green, or blue;
the fluorescent color of the second fluorescent group is red, green, or blue;
optionally, a combination of the first fluorescent signal and the second fluorescent signal is a combination formed of red and green; or a combination of the first fluorescent signal and the second fluorescent signal is a combination formed of red and blue;
optionally, the fluorescent group whose fluorescent color is green includes Atto532, Alexa Fluor532, Atto Rho6G, or CY3;
the fluorescent group whose fluorescent color is red comprises: Atto647N, CY5, GM656, or Alexa Fluor647;
the fluorescent group whose fluorescent color is blue comprises: Atto390, Alexa Fluor405, or coumarin;
optionally, the labeling substance comprises n fluorescent groups, where n is 1 or 2;
optionally, a combination of the specific binding label and the ligand is selected from at least one of streptavidin (SA)-Biotin combination, Biotin-streptavidin (SA) combination, Biotin (SA)-avidin combination, or antibody-antigen combination;
optionally, a combination of specific binding label and the ligand is selected from Biotin-streptavidin combination.
5. The two-color sequencing method according to claim 4, characterized in that the four types of reversible terminator include at least one of the following Combinations 1 to 14:
Combination 1: adenine nucleotide or derivative thereof-N3-Atto647N, guanine nucleotide or derivative thereof-N3-SS-Atto647N, thymine nucleotide or derivative thereof-N3-Biotin, and cytosine nucleotide or derivative thereof-N3-Atto532;
Combination 2: adenine nucleotide or derivative thereof-N3-CY5, guanine nucleotide or derivative thereof-N3-SS-Atto647N, thymine nucleotide or derivative thereof-N3-Biotin, and cytosine nucleotide or derivative thereof-N3-Atto532;
Combination 3: adenine nucleotide or derivative thereof-N3-CY5, guanine nucleotide or derivative thereof-N3-SS-CY5, thymine nucleotide or derivative thereof-N3-Biotin, and cytosine nucleotide or derivative thereof-N3-Atto532;
Combination 4: adenine nucleotide or derivative thereof-N3-Atto647N, guanine nucleotide or derivative thereof-N3-SS-CY5, thymine nucleotide or derivative thereof-N3-Biotin, and cytosine nucleotide or derivative thereof-N3-Atto532;
Combination 5: adenine nucleotide or derivative thereof-N3-Atto647N, guanine nucleotide or derivative thereof-N3-SS-Atto647N, thymine nucleotide or derivative thereof-N3-Atto532-Biotin, and cytosine nucleotide or derivative thereof-N3-Atto532;
Combination 6: adenine nucleotide or derivative thereof-N3-Atto647N, guanine nucleotide or derivative thereof-N3-SS-CY5, cytosine nucleotide or derivative thereof-N3-Atto532-Biotin, and cytosine nucleotide or derivative thereof-N3-Atto532;
Combination 7: adenine nucleotide or derivative thereof-N3-CY5, guanine nucleotide or derivative thereof-N3-SS-Atto647N, thymine nucleotide or derivative thereof-N3-Atto532-Biotin, and cytosine nucleotide or derivative thereof-N3-Atto532;
Combination 8: adenine nucleotide or derivative thereof-N3-CY5, guanine nucleotide or derivative thereof-N3-SS-CY5, thymine nucleotide or derivative thereof-N3-Atto532-Biotin, and cytosine nucleotide or derivative thereof-N3-Atto532;
Combination 9: adenine nucleotide or derivative thereof-N3-Atto647N, guanine nucleotide or derivative thereof-N3-SS-Atto532, thymine nucleotide or derivative thereof-N3-Biotin, and cytosine nucleotide or derivative thereof-N3-Atto532;
Combination 10: adenine nucleotide or derivative thereof-N3-CY5, guanine nucleotide or derivative thereof-N3-SS-Atto532, thymine nucleotide or derivative thereof-N3-Biotin, and cytosine nucleotide or derivative thereof-N3-Atto532;
Combination 11: adenine nucleotide or derivative thereof-N3-Atto532, guanine nucleotide or derivative thereof-N3-SS-Atto532, cytosine nucleotide or derivative thereof-N3-Biotin, and cytosine nucleotide or derivative thereof-N3-Atto647N;
Combination 12: adenine nucleotide or derivative thereof-N3-Atto532, guanine nucleotide or derivative thereof-N3-SS-Atto532, thymine nucleotide or derivative thereof-N3-Biotin, and cytosine nucleotide or derivative thereof-N3-CY5;
Combination 13: adenine nucleotide or derivative thereof-N3-Atto532, guanine nucleotide or derivative thereof-N3-SS-Atto532, cytosine nucleotide or derivative thereof-N3-Atto647N-Biotin, and cytosine nucleotide or derivative thereof-N3-Atto647N;
Combination 14: adenine nucleotide or derivative thereof-N3-Atto532, guanine nucleotide or derivative thereof-N3-SS-Atto532, thymine cytosine nucleotide or derivative thereof-N3-CY5-Biotin, and cytosine nucleotide or derivative thereof-N3-CY5;
optionally,
in Combinations 1 to 14, the cleavable group —N3- in any one of the reversible terminators not having the specific binding label is replaced with —N3-SS—, and the cleavable group —N3-SS— in any one of the reversible terminators not having the specific binding label is replaced with —N3-; and/or
In Combinations 1 to 14, —N3-SS— is replaced with —SS—.
6. The two-color sequencing method according to claim 5, characterized in that,
in Combinations 1 to 14, CY5 and/or Atto647N are/is replaced with GM656 or Alexa Fluor647; and/or Atto532 and/or Alexa Fluor532 are/is replaced with Atto Rho6G or CY3; or
in Combinations 1 to 14, CY5 and/or Atto647N are/is replaced with one of the blue light dyes.
7. The two-color sequencing method according to claim 5, wherein the reversible terminators are Combination 1;
optionally, in the Combination 1, the use concentration of adenine nucleotide or derivative thereof-N3-Atto647N is from 10 nmol/L to 700 nmol/L, the use concentration of thymine nucleotide-N3-Biotin is from 5 nmol/L to 60 nmol/L, the use concentration of cytosine nucleotide or derivative thereof-N3-Atto532 is from 10 nmol/L to 700 nmol/L, and the use concentration of guanine nucleotide or derivative thereof-N3-SS-Atto647N is from 0.05 nmol/L to 5 nmol/L.
8. The two-color sequencing method according to claim 3, wherein,
the cleavable groups in the four types of reversible terminator are the same and are any one of the first cleavable group or the second cleavable group;
the first signal, the second signal, the third signal, the sixth signal, and the seventh signal satisfy condition 3) or condition 4), and the structures of the four types of reversible terminator satisfy the following:
the structure of the first reversible terminator is: first nucleotide or derivative thereof-first cleavable group or second cleavable group-first fluorescent group;
the structure of the second reversible terminator is: second nucleotide or derivative thereof-first cleavable group or second cleavable group-specific binding label; or second nucleotide or derivative thereof-first cleavable group or second cleavable group-first fluorescent group or second fluorescent group-specific binding label;
the structure of the third reversible terminator is: third nucleotide or derivative thereof-first cleavable group or second cleavable group-specific binding label; or third nucleotide or derivative thereof-first cleavable group or second cleavable group-second fluorescent group or first fluorescent group-specific binding label;
the structure of the fourth reversible terminator is: fourth nucleotide or derivative thereof-first cleavable group or second cleavable group-second fluorescent group;
wherein the first nucleotide or derivative thereof, the second nucleotide or derivative thereof, the third nucleotide or derivative thereof, and the fourth nucleotide or derivative thereof are selected from one of adenine or derivative thereof, thymine or derivative thereof, cytosine or derivative thereof, or guanine or derivative thereof, and the first nucleotide or derivative thereof, the second nucleotide or derivative thereof, the third nucleotide or derivative thereof, and the fourth nucleotide or derivative thereof are different from each other,
optionally, the first fluorescent group and the second fluorescent group are each independently selected from: one or combinations of more of Atto532, Atto647N, Atto390, Atto Rho6G, Alexa Fluor405, Alexa Fluor532, GM656, Alexa Fluor647, CY5, IF700, ROX, CY3, CY5 rhodamine dye, cyanine dye, coumarin dye, or phycoerythrin;
optionally, the fluorescent color of the first fluorescent group is red, green, or blue;
the fluorescent color of the second fluorescent group is red, green, or blue;
optionally, a combination of the first fluorescent signal and the second fluorescent signal is a combination formed of red and green; or a combination of the first fluorescent signal and the second fluorescent signal is a combination formed of red and blue;
optionally, the fluorescent group whose fluorescent color is green includes Atto532, Alexa Fluor532, Atto Rho6G, or CY3;
the fluorescent group whose fluorescent color is red includes: Atto647N, CY5, GM656, or Alexa Fluor647;
the fluorescent group whose fluorescent color is blue includes: Atto390, Alexa Fluor405, or coumarin;
optionally, the labeling substance comprises n fluorescent groups, wherein n is 1 or 2; optionally, a combination of the specific binding label and the ligand is selected from at least one of streptavidin (SA)-Biotin combination, Biotin-streptavidin (SA) combination, Biotin-avidin combination, or antibody-antigen;
optionally, a combination of specific binding label and the ligand is selected from Biotin-streptavidin (SA) combination.
9. The two-color sequencing method according to claim 8, wherein the reversible terminators include at least one group shown in the following Combinations 15 to 25:
Combination 15: adenine nucleotide or derivative thereof-N3-Atto647N, guanine nucleotide or derivative thereof-N3-Biotin, thymine nucleotide or derivative thereof-N3-Atto532-Biotin, and cytosine nucleotide or derivative thereof-N3-Atto532;
Combination 16: adenine nucleotide or derivative thereof-N3-CY5, guanine nucleotide or derivative thereof-N3-Biotin, thymine nucleotide or derivative thereof-N3-Atto532-Biotin, and cytosine nucleotide or derivative thereof-N3-Atto532;
Combination 17: adenine nucleotide or derivative thereof-N3-Atto647N, guanine nucleotide or derivative thereof-N3-Biotin, thymine cytosine nucleotide or derivative thereof-N3-Atto647N-Biotin, and cytosine nucleotide or derivative thereof-N3-Atto532;
Combination 18: adenine nucleotide or derivative thereof-N3-CY5, guanine nucleotide or derivative thereof-N3-Biotin, thymine nucleotide or derivative thereof-N3-Atto647N-Biotin, and cytosine nucleotide or derivative thereof-N3-Atto532;
Combination 19: adenine nucleotide or derivative thereof-N3-Atto647N, guanine nucleotide or derivative thereof-N3-Biotin, thymine nucleotide or derivative thereof-N3-CY5-Biotin, and cytosine nucleotide or derivative thereof-N3-Atto532;
Combination 20: adenine nucleotide or derivative thereof-N3-Atto532, guanine nucleotide or derivative thereof-N3-Biotin, thymine cytosine nucleotide or derivative thereof-N3-Atto532-Biotin, and cytosine nucleotide or derivative thereof-N3-Atto647N;
Combination 21: adenine nucleotide or derivative thereof-N3-Atto532, guanine nucleotide or derivative thereof-N3-Biotin, thymine cytosine nucleotide or derivative thereof-N3-Atto532-Biotin, and cytosine nucleotide or derivative thereof-N3-CY5;
Combination 22: adenine nucleotide or derivative thereof-N3-Atto647N, guanine nucleotide or derivative thereof-N3-Biotin, thymine cytosine nucleotide or derivative thereof-N3-Biotin, and cytosine nucleotide or derivative thereof-N3-Atto532;
Combination 23: adenine nucleotide or derivative thereof-N3-CY5, guanine nucleotide or derivative thereof-N3-Biotin, thymine nucleotide or derivative thereof-N3-Biotin, and cytosine nucleotide or derivative thereof-N3-Atto532;
Combination 24: adenine nucleotide or derivative thereof-N3-Atto532, guanine nucleotide or derivative thereof-N3-Biotin, thymine cytosine nucleotide or derivative thereof-N3-Biotin, and cytosine nucleotide or derivative thereof-N3-Atto647N;
Combination 25: adenine nucleotide or derivative thereof-N3-Atto532, guanine nucleotide or derivative thereof-N3-Biotin, thymine cytosine nucleotide or derivative thereof-N3-Biotin, and cytosine nucleotide or derivative thereof-N3-CY5; 5
optionally, in Combinations 15 to 25, the cleavable group —N3- is replaced with —SS—.
10. The two-color sequencing method according to claim 9, wherein,
in Combinations 15 to 25, CY5 and/or Atto647N are/is replaced with GM656 or Alexa Fluor647; and/or Atto532 and/or Alexa Fluor532 are/is replaced with Atto Rho6G or CY3; or
in Combinations 15 to 25, CY5 and/or Atto647N are/is replaced with one of the blue light dyes.
11. The two-color sequencing method according to claim 10, wherein,
the reversible terminators are Combination 17;
optionally, in the Combination 17, the use concentration of adenine nucleotide or derivative thereof-N3-Atto647N is from 10 nmol/L to 700 nmol/L, the use concentration of thymine or derivative thereof-N3-Atto532-Biotin is from 5 nmol/L to 60 nmol/L, the use concentration of cytosine nucleotide or derivative thereof-N3-Atto532 is from 10 nmol/L to 700 nmol/L, and the use concentration of cytosine nucleotide or derivative thereof-N3-Bitoin is from 0.05 nmol/L to 5 nmol/L.
12. The two-color sequencing method according to any one of claims 1-11, wherein,
the binding reaction comprises adding the labeling substance;
optionally, the time of the binding reaction is greater than or equal to 20 s;
the cleavage reagent for cleaving the first cleavable group is an organophosphorus cleavage;
optionally, in the organophosphorus cleavage reagent, the organophosphorus is selected from any one or more of tris(3-hydroxypropyl)phosphine, tris(2-carboxyethyl)phosphine, trimethylolphosphine, triphenylphosphine, or tris(2-hydroxyethyl)phosphine;
optionally, in the organophosphorus cleavage reagent, the organophosphorus is tris(3-hydroxypropyl)phosphine;
optionally, the use concentration of the tris(3-hydroxypropyl)phosphine is from 10 mmol/L to 100 mmol/L;
the cleavage reagent for cleaving the second cleavable group is a reducing agent;
optionally, in the reducing agent, the reducing agent is selected from DTT, glutathione, and/or β-SS-ethanol;
optionally, the reducing agent is glutathione;
optionally, the use concentration of the glutathione is from 10 mmol/L to 100 mmol/L.
13. A kit, characterized in that the kit comprises four types of reversible terminator, a first cleavage reagent and/or a second cleavage reagent;
the four types of reversible terminator independently being: a first reversible terminator, a second reversible terminator, a third reversible terminator, and a fourth reversible terminator; the four types of reversible terminator each independently comprising: a nucleotide or derivative thereof, a labeling moiety, and a first cleavable group and/or a second cleavable group linking the nucleotide or derivative thereof and the labeling moiety, the nucleotide or derivative thereof being selected from adenine or derivative thereof, thymine or derivative thereof, cytosine or derivative thereof, or guanine or derivative thereof,
wherein,
the labeling moiety is selected from: a fluorescent group and/or a specific binding label, the specific binding label being configured to bind to a labeling substance, and the labeling substance comprising a ligand and a fluorescent group bound to the ligand;
when the reversible terminator comprises both the first cleavable group and the second cleavable group, the first cleavable group and the second cleavable group are different, and the second cleavable group has a higher cleavability than the first cleavable group;
the first cleavage reagent is a reagent for cleaving the first cleavable group or the second cleavable group;
the second cleavage reagent is a reagent for cleaving the first cleavable group or the second cleavable;
optionally, the cleavage reagent for cleaving the first cleavable group is an organophosphorus cleavage;
optionally, in the organophosphorus cleavage reagent, the organophosphorus is selected from any one or more of tris(3-hydroxypropyl)phosphine, tris(2-carboxyethyl)phosphine, trimethylolphosphine, triphenylphosphine, or tris(2-hydroxyethyl)phosphine;
optionally, in the organophosphorus cleavage reagent, the organophosphorus is tris(3-hydroxypropyl)phosphine;
optionally, the use concentration of the tris(3-hydroxypropyl)phosphine is from 10 mmol/L to 100 mmol/L;
optionally, the cleavage reagent for cleaving the second cleavable group is a reducing agent;
optionally, in the reducing agent, the reducing agent is selected from DTT, glutathione, and/or β-SS-ethanol;
optionally, the reducing agent is glutathione;
optionally, the use concentration of the glutathione is from 10 mmol/L to 100 mmol/L.
14. The kit according to claim 13, wherein,
one or more of the four types of reversible terminator comprise(s) the first cleavable group, and one or more of the four types of reversible terminator comprise(s) the second cleavable group;
optionally,
the structure of the first reversible terminator is: first nucleotide or derivative thereof-first cleavable group-first fluorescent group,
the structure of the second reversible terminator is one of the following scenarios: second nucleotide or derivative thereof-first cleavable group-second cleavable group-first fluorescent group or second fluorescent group; or second nucleotide or derivative thereof-second cleavable group-first fluorescent group or second fluorescent group;
the structure of the third reversible terminator is one of the following scenarios: second nucleotide or derivative thereof-first cleavable group-specific binding label; or third nucleotide or derivative thereof-first cleavable group-first fluorescent group or second fluorescent group-specific binding label;
the structure of the fourth reversible terminator is: fourth nucleotide or derivative thereof-first cleavable group-second fluorescent group;
the second reversible terminator and the third reversible terminator do not comprise the specific binding label at the same time;
or
the cleavable groups in the four types of reversible terminator are the same and are any one of the first cleavable group or the second cleavable group;
the structure of the first reversible terminator is: first nucleotide or derivative thereof-first cleavable group or second cleavable group-first fluorescent group;
the structure of the second reversible terminator is: second nucleotide or derivative thereof-first cleavable group or second cleavable group-specific binding label; or second nucleotide or derivative thereof-first cleavable group or second cleavable group-first fluorescent group or second fluorescent group-specific binding label;
the structure of the third reversible terminator is: third nucleotide or derivative thereof-first cleavable group or second cleavable group-specific binding label; or third nucleotide or derivative thereof-first cleavable group or second cleavable group-second fluorescent group or first fluorescent group-specific binding label;
the structure of the fourth reversible terminator is: fourth nucleotide or derivative thereof-first cleavable group or second cleavable group-second fluorescent group;
wherein,
the first nucleotide or derivative thereof, the second nucleotide or derivative thereof, the third nucleotide or derivative thereof, and the fourth nucleotide or derivative thereof are selected from one of adenine or derivative thereof, thymine or derivative thereof, cytosine or derivative thereof, or guanine or derivative thereof, and the first nucleotide or derivative thereof, the second nucleotide or derivative thereof, the third nucleotide or derivative thereof, and the fourth nucleotide or derivative thereof are different from each other,
optionally, the first cleavable group is selected from groups comprising at least one of —N₃—, —CH₂F, —C(O)NHBu, —CHF₂, —C(O)OH, —C6 and/or —ArC6, or disulfide;
optionally, the first cleavable group is selected from at least one of —N₃—, —CH₂F, —C(O)NHBu, —CHF₂, —C(O)OH, —C₆ and/or —ArC₆, or disulfide;
optionally, the first cleavable group is —N3- or disulfide;
optionally, the second cleavable group is selected from groups comprising disulfide;
optionally, the second cleavable group is disulfide bond —SS—;
optionally, the fluorescent group is selected from the first fluorescent group and the second fluorescent group, which are distinguishable in color;
optionally, the first fluorescent group and the second fluorescent group are each independently selected from: one or combinations of more of Atto532, Atto647N, Atto390, Atto Rho6G, Alexa Fluor405, Alexa Fluor532, GM656, Alexa Fluor647, CY5, IF700, ROX, CY3, CY5 rhodamine dye, cyanine dye, coumarin dye, or phycoerythrin;
optionally, the fluorescent color of the first fluorescent group is red, green, or blue;
the fluorescent color of the second fluorescent group is red, green, or blue;
optionally, a combination of the first fluorescent signal and the second fluorescent signal is a combination formed of red and green; or a combination of the first fluorescent signal and the second fluorescent signal is a combination formed of red and blue;
optionally, the fluorescent group whose fluorescent color is green includes Atto532, Alexa Fluor532, Atto Rho6G, or CY3;
the fluorescent group whose fluorescent color is red includes: Atto647N, CY5, GM656, or Alexa Fluor647;
the fluorescent group whose fluorescent color is blue includes: Atto390, Alexa Fluor405, or coumarin;
optionally, the labeling substance comprises n fluorescent groups, wherein n is 1 or 2;
optionally, a combination of specific binding label and the ligand is selected from at least one of Biotin-streptavidin (SA) combination, Biotin (SA)-streptavidin (Biotin) combination, Biotin-avidin combination, or antibody-antigen;
optionally, a combination of specific binding label and the ligand is selected from Biotin-streptavidin (SA) combination.
15. The kit according to claim 14, wherein the reversible terminators include at least one of the following Combinations 1 to 14 or Combinations 15 to 25:
Combination 1: adenine nucleotide or derivative thereof-N3-Atto647N, guanine nucleotide or derivative thereof-N3-SS-Atto647N, thymine nucleotide or derivative thereof-N3-Biotin, and cytosine nucleotide or derivative thereof-N3-Atto532;
Combination 2: adenine nucleotide or derivative thereof-N3-CY5, guanine nucleotide or derivative thereof-N3-SS-Atto647N, thymine nucleotide or derivative thereof-N3-Biotin, and cytosine nucleotide or derivative thereof-N3-Atto532;
Combination 3: adenine nucleotide or derivative thereof-N3-CY5, guanine nucleotide or derivative thereof-N3-SS-CY5, thymine nucleotide or derivative thereof-N3-Biotin, and cytosine nucleotide or derivative thereof-N3-Atto532;
Combination 4: adenine nucleotide or derivative thereof-N3-Atto647N, guanine nucleotide or derivative thereof-N3-SS-CY5, thymine nucleotide or derivative thereof-N3-Biotin, and cytosine nucleotide or derivative thereof-N3-Atto532;
Combination 5: adenine nucleotide or derivative thereof-N3-Atto647N, guanine nucleotide or derivative thereof-N3-SS-Atto647N, thymine nucleotide or derivative thereof-N3-Atto532-Biotin, and cytosine nucleotide or derivative thereof-N3-Atto532;
Combination 6: adenine nucleotide or derivative thereof-N3-Atto647N, guanine nucleotide or derivative thereof-N3-SS-CY5, cytosine nucleotide or derivative thereof-N3-Atto532-Biotin, and cytosine nucleotide or derivative thereof-N3-Atto532;
Combination 7: adenine nucleotide or derivative thereof-N3-CY5, guanine nucleotide or derivative thereof-N3-SS-Atto647N, thymine nucleotide or derivative thereof-N3-Atto532-Biotin, and cytosine nucleotide or derivative thereof-N3-Atto532;
Combination 8: adenine nucleotide or derivative thereof-N3-CY5, guanine nucleotide or derivative thereof-N3-SS-CY5, thymine nucleotide or derivative thereof-N3-Atto532-Biotin, and cytosine nucleotide or derivative thereof-N3-Atto532;
Combination 9: adenine nucleotide or derivative thereof-N3-Atto647N, guanine nucleotide or derivative thereof-N3-SS-Atto532, thymine nucleotide or derivative thereof-N3-Biotin, and cytosine nucleotide or derivative thereof-N3-Atto532;
Combination 10: adenine nucleotide or derivative thereof-N3-CY5, guanine nucleotide or derivative thereof-N3-SS-Atto532, thymine nucleotide or derivative thereof-N3-Biotin, and cytosine nucleotide or derivative thereof-N3-Atto532;
Combination 11: adenine nucleotide or derivative thereof-N3-Atto532, guanine nucleotide or derivative thereof-N3-SS-Atto532, cytosine nucleotide or derivative thereof-N3-Biotin, and cytosine nucleotide or derivative thereof-N3-Atto647N;
Combination 12: adenine nucleotide or derivative thereof-N3-Atto532, guanine nucleotide or derivative thereof-N3-SS-Atto532, thymine nucleotide or derivative thereof-N3-Biotin, and cytosine nucleotide or derivative thereof-N3-CY5;
Combination 13: adenine nucleotide or derivative thereof-N3-Atto532, guanine nucleotide or derivative thereof-N3-SS-Atto532, cytosine nucleotide or derivative thereof-N3-Atto647N-Biotin, and cytosine nucleotide or derivative thereof-N3-Atto647N;
Combination 14: adenine nucleotide or derivative thereof-N3-Atto532, guanine nucleotide or derivative thereof-N3-Atto532, thymine cytosine nucleotide or derivative thereof-N3-CY5-Biotin, and cytosine nucleotide or derivative thereof-N3-CY5;
Combination 15: adenine nucleotide or derivative thereof-N3-Atto647N, guanine nucleotide or derivative thereof-N3-Biotin, thymine nucleotide or derivative thereof-N3-Atto532-Biotin, and cytosine nucleotide or derivative thereof-N3-Atto532;
Combination 16: adenine nucleotide or derivative thereof-N3-CY5, guanine nucleotide or derivative thereof-N3-Biotin, thymine nucleotide or derivative thereof-N3-Atto532-Biotin, and cytosine nucleotide or derivative thereof-N3-Atto532;
Combination 17: adenine nucleotide or derivative thereof-N3-Atto647N, guanine nucleotide or derivative thereof-N3-Biotin, thymine cytosine nucleotide or derivative thereof-N3-Atto647N-Biotin, and cytosine nucleotide or derivative thereof-N3-Atto532;
Combination 18: adenine nucleotide or derivative thereof-N3-CY5, guanine nucleotide or derivative thereof-N3-Biotin, thymine nucleotide or derivative thereof-N3-Atto647N-Biotin, and cytosine nucleotide or derivative thereof-N3-Atto532;
Combination 19: adenine nucleotide or derivative thereof-N3-Atto647N, guanine nucleotide or derivative thereof-N3-Biotin, thymine nucleotide or derivative thereof-N3-CY5-Biotin, and cytosine nucleotide or derivative thereof-N3-Atto532;
Combination 20: adenine nucleotide or derivative thereof-N3-Atto532, guanine nucleotide or derivative thereof-N3-Biotin, thymine cytosine nucleotide or derivative thereof-N3-Atto532-Biotin, and cytosine nucleotide or derivative thereof-N3-Atto647N;
Combination 21: Adenine nucleotide or derivative thereof-N3-Atto532, guanine nucleotide or derivative thereof-N3-Biotin, thymine cytosine nucleotide or derivative thereof-N3-Atto532-Biotin, and cytosine nucleotide or derivative thereof-N3-CY5;
Combination 22: adenine nucleotide or derivative thereof-N3-Atto647N, guanine nucleotide or derivative thereof-N3-Biotin, thymine cytosine nucleotide or derivative thereof-N3-Biotin, and cytosine nucleotide or derivative thereof-N3-Atto532;
Combination 23: adenine nucleotide or derivative thereof-N3-Atto647N, guanine nucleotide or derivative thereof-N3-Biotin, thymine cytosine nucleotide or derivative thereof-N3-Biotin, and cytosine nucleotide or derivative thereof-N3-Atto532;
Combination 24: adenine nucleotide or derivative thereof-N3-Atto532, guanine nucleotide or derivative thereof-N3-Biotin, thymine cytosine nucleotide or derivative thereof-N3-Biotin, and cytosine nucleotide or derivative thereof-N3-Atto647N;
Combination 25: adenine nucleotide or derivative thereof-N3-Atto532, guanine nucleotide or derivative thereof-N3-Biotin, thymine cytosine nucleotide or derivative thereof-N3-Biotin, and cytosine nucleotide or derivative thereof-N3-CY5,
optionally, in Combinations 1 to 14, the cleavable group —N3- in any one of the reversible terminators not having the specific binding label is replaced with —N3-SS—, and the cleavable group —N3-SS— in any one of the reversible terminators not having the specific binding label is replaced with —N3-; and/or
in Combinations 1 to 14, —N3- linked to cytosine or derivative thereof or pyrimidine or derivative thereof is replaced with —N3-SS—, and —N3-SS— linked to guanine nucleotide or derivative thereof is replaced with —N3-; and/or
in Combinations 15 to 25, the cleavable group —N3- is replaced with —SS—.
16. The kit according to claim 15, wherein in Combinations 1 to 25, CY5 and/or Atto647N are/is replaced with GM656 or Alexa Fluor647; and/or Atto532 and/or Alexa Fluor532 are/is replaced with Atto Rho6G or CY3; or
in Combinations 1 to 25, CY5 and/or Atto647N are/is replaced with one of the blue light dyes.
optionally, the reversible terminators are Combination 1;
optionally, in the Combination 1, the use concentration of adenine nucleotide or derivative thereof-N3-Atto647N is from 10 nmol/L to 700 nmol/L, the use concentration of thymine nucleotide or derivative thereof-N3-Biotin/SA-CY3 is from 5 nmol/L to 60 nmol/L, the use concentration of cytosine nucleotide or derivative thereof-N3-Atto532 is from 10 nmol/L to 700 nmol/L, and the use concentration of guanine nucleotide or derivative thereof-N3-SS-Atto647N is from 0.05 nmol/L to 5 nmol/L.

What is claimed is:

1. A two-color sequencing method, wherein comprises the following steps:
(a) allowing four types of reversible terminator to undergo polymerization reactions with nucleic acid molecules attached to a surface, the four types of reversible terminator each independently comprising: a nucleotide or derivative thereof, a labeling moiety, and a first cleavable group linking the nucleotide or derivative thereof and the labeling moiety, wherein the labeling moiety is selected from a fluorescent group and/or a specific binding label, at least two of the four types of modified nucleotide carry a first fluorescent group and a second fluorescent group, which are optically distinguishable, respectively, one or two of the four types of modified nucleotide carry the specific binding label, and the specific binding label is capable of specifically binding to a corresponding ligand;
(b) exciting the fluorescent group to luminesce, and collecting a luminescence signal to obtain a first image;
(c) adding a labeling substance, the labeling substance comprising the ligand and a fluorescent group bound to the ligand, and allowing the ligand of the labeling substance to bind to the specific binding label, wherein the fluorescent group bound to the ligand is selected from the first fluorescent group or the second fluorescent group;
(d) exciting the fluorescent group to luminesce, and collecting a luminescence signal from the fluorescent group to obtain a second image; and
(e) determining the types of reversible terminator participating in the polymerization reactions on the basis of the luminescence signals and a change thereof at the same position in the first image and the second image.

2. The two-color sequencing method according to claim 1, wherein the labeling moieties of two of the four types of modified nucleotide are the first fluorescent group and the second fluorescent group, which are optically distinguishable, respectively, and the labeling moieties of the other two of the four types of modified nucleotide are specific binding labels with different modes of specific binding, and
in step (c), the labeling substance comprises two types, and the two types of labeling substance carry the first fluorescent group and the second fluorescent group, respectively; or
two of the four types of modified nucleotide each independently carry the first fluorescent group, one of the four types of modified nucleotide carries the second fluorescent group, and the other of the four types of modified nucleotide carries the specific binding label, and
in step (c), the labeling substance comprises one type, and the labeling substance carries the second fluorescent group.

3. The two-color sequencing method according to claim 1, wherein three of the four types of modified nucleotide carry the first fluorescent group and the second fluorescent group, which are optically distinguishable, respectively, the other of the four types of modified nucleotide carries the specific binding label, and one of the two types of modified nucleotide carrying the same fluorescent group comprises a second cleavable group, the second cleavable group being so located as to link the first cleavable group and the labeling moiety, and the second cleavable group having a higher cleavability than the first cleavable group, and
step (c) further comprises: performing a cleavage reaction on the second cleavable group.

4. The two-color sequencing method according to claim 1, wherein determining the types of reversible terminator participating in the polymerization reactions on the basis of the luminescence signals and a change thereof at the same position in the first image and the second image comprises:
with respect to a first position where the optical signals remain unchanged in both the first image and the second image and are both indicative of the optical signal of the first fluorescent group, determining the reversible terminator participating in the polymerization reaction at that position to be a first reversible terminator;
with respect to a second position where the optical signals remain unchanged in both the first image and the second image and are both indicative of the optical signal of the second fluorescent group, determining the reversible terminator participating in the polymerization reaction at that position to be a second reversible terminator; and
with respect to a third position and a fourth position where the optical signals become changed but follow different patterns of change between the first image and the second image, determining the reversible terminators participating in the polymerization reactions at those two positions to be a third reversible terminator and a fourth reversible terminator.

5. The two-color sequencing method according to claim 4, wherein the structures of the four types of reversible terminator satisfy one or more of the following combinations (1)-(4):
(1) the structure of the first reversible terminator is: first nucleotide or derivative thereof-first cleavable group-first fluorescent group,
the structure of the second reversible terminator is: second nucleotide or derivative thereof-first cleavable group-specific binding label,
the structure of the third reversible terminator is: third nucleotide or derivative thereof-first cleavable group-specific binding label, and
the structure of the fourth reversible terminator is: fourth nucleotide or derivative thereof-first cleavable group-second fluorescent group;
(2) the structure of the first reversible terminator is: first nucleotide or derivative thereof-first cleavable group-first fluorescent group,
the structure of the second reversible terminator is: second nucleotide or derivative thereof-first cleavable group-specific binding label,
the structure of the third reversible terminator is: third nucleotide or derivative thereof-first cleavable group-specific binding label, and
the structure of the fourth reversible terminator is: fourth nucleotide or derivative thereof-first cleavable group-second fluorescent group-specific binding label;
(3) the structure of the first reversible terminator is: first nucleotide or derivative thereof-first cleavable group-first fluorescent group,
the structure of the second reversible terminator is: second nucleotide or derivative thereof-first cleavable group-specific binding label,
the structure of the third reversible terminator is: third nucleotide or derivative thereof-first cleavable group-second fluorescent group, and
the structure of the fourth reversible terminator is: fourth nucleotide or derivative thereof-first cleavable group-second cleavable group-first fluorescent group-specific binding label;
(4) the structure of the first reversible terminator is: first nucleotide or derivative thereof-first cleavable group-first fluorescent group,
the structure of the second reversible terminator is: second nucleotide or derivative thereof-first cleavable group-second fluorescent group-specific binding label,
the structure of the third reversible terminator is: third nucleotide or derivative thereof-first cleavable group-second fluorescent group, and
the structure of the fourth reversible terminator is: fourth nucleotide or derivative thereof-first cleavable group-second cleavable group-first fluorescent group;
wherein in each combination, the first nucleotide or derivative thereof, the second nucleotide or derivative thereof, the third nucleotide or derivative thereof, and the fourth nucleotide or derivative thereof are selected from one of adenine or a derivative thereof, thymine or a derivative thereof, cytosine or a derivative thereof, or guanine or a derivative thereof, and the first nucleotide or a derivative thereof, the second nucleotide or a derivative thereof, the third nucleotide or a derivative thereof, and the fourth nucleotide or a derivative thereof are different from each other.

6. The two-color sequencing method according to claim 5, wherein the first cleavable group is selected from groups comprising at least one of —N3-, —CH₂F, —C(O)NHBu, —CHF₂, —C(O)OH, —C6, —ArC6, and/or a disulfide bond; or
the four types of reversible terminator each independently comprise the first cleavable group and/or the second cleavable group, the first cleavable group is —N₃—, and the second cleavable group is selected from groups comprising a disulfide bond.

7. The two-color sequencing method according to claim 5, wherein the first fluorescent group and the second fluorescent group are each independently selected from one of a red fluorescent group, a green fluorescent group, and a blue fluorescent group.

8. The two-color sequencing method according to claim 1, wherein the specific binding label is selected from at least one of biotin, digoxin, hapten, or functional derivatives thereof.

9. The two-color sequencing method according to claim 1, wherein the reversible terminators comprise at least one group shown in the following Combinations 1 to 25:
Combination 1: adenine nucleotide or derivative thereof-N3-Atto647N, guanine nucleotide or derivative thereof-N3-SS-Atto647N, thymine nucleotide or derivative thereof-N3-Biotin, and cytosine nucleotide or derivative thereof-N3-Atto532;
Combination 2: adenine nucleotide or derivative thereof-N3-CY5, guanine nucleotide or derivative thereof-N3-SS-Atto647N, thymine nucleotide or derivative thereof-N3-Biotin, and cytosine nucleotide or derivative thereof-N3-Atto532;
Combination 3: adenine nucleotide or derivative thereof-N3-CY5, guanine nucleotide or derivative thereof-N3-SS-CY5, thymine nucleotide or derivative thereof-N3-Biotin, and cytosine nucleotide or derivative thereof-N3-Atto532;
Combination 4: adenine nucleotide or derivative thereof-N3-Atto647N, guanine nucleotide or derivative thereof-N3-SS-CY5, thymine nucleotide or derivative thereof-N3-Biotin, and cytosine nucleotide or derivative thereof-N3-Atto532;
Combination 5: adenine nucleotide or derivative thereof-N3-Atto647N, guanine nucleotide or derivative thereof-N3-SS-Atto647N, thymine nucleotide or derivative thereof-N3-Atto532-Biotin, and cytosine nucleotide or derivative thereof-N3-Atto532;
Combination 6: adenine nucleotide or derivative thereof-N3-Atto647N, guanine nucleotide or derivative thereof-N3-SS-CY5, cytosine nucleotide or derivative thereof-N3-Atto532-Biotin, and cytosine nucleotide or derivative thereof-N3-Atto532;
Combination 7: adenine nucleotide or derivative thereof-N3-CY5, guanine nucleotide or derivative thereof-N3-SS-Atto647N, thymine nucleotide or derivative thereof-N3-Atto532-Biotin, and cytosine nucleotide or derivative thereof-N3-Atto532;
Combination 8: adenine nucleotide or derivative thereof-N3-CY5, guanine nucleotide or derivative thereof-N3-SS-CY5, thymine nucleotide or derivative thereof-N3-Atto532-Biotin, and cytosine nucleotide or derivative thereof-N3-Atto532;
Combination 9: adenine nucleotide or derivative thereof-N3-Atto647N, guanine nucleotide or derivative thereof-N3-SS-Atto532, thymine nucleotide or derivative thereof-N3-Biotin, and cytosine nucleotide or derivative thereof-N3-Atto532;
Combination 10: adenine nucleotide or derivative thereof-N3-CY5, guanine nucleotide or derivative thereof-N3-SS-Atto532, thymine nucleotide or derivative thereof-N3-Biotin, and cytosine nucleotide or derivative thereof-N3-Atto532;
Combination 11: adenine nucleotide or derivative thereof-N3-Atto532, guanine nucleotide or derivative thereof-N3-SS-Atto532, cytosine nucleotide or derivative thereof-N3-Biotin, and cytosine nucleotide or derivative thereof-N3-Atto647N;
Combination 12: adenine nucleotide or derivative thereof-N3-Atto532, guanine nucleotide or derivative thereof-N3-SS-Atto532, thymine nucleotide or derivative thereof-N3-Biotin, and cytosine nucleotide or derivative thereof-N3-CY5;
Combination 13: adenine nucleotide or derivative thereof-N3-Atto532, guanine nucleotide or derivative thereof-N3-SS-Atto532, cytosine nucleotide or derivative thereof-N3-Atto647N-Biotin, and cytosine nucleotide or derivative thereof-N3-Atto647N;
Combination 14: adenine nucleotide or derivative thereof-N3-Atto532, guanine nucleotide or derivative thereof-N3-SS-Atto532, thymine cytosine nucleotide or derivative thereof-N3-CY5-Biotin, and cytosine nucleotide or derivative thereof-N3-CY5;
Combination 15: adenine nucleotide or derivative thereof-N3-Atto647N, guanine nucleotide or derivative thereof-N3-Biotin, thymine nucleotide or derivative thereof-N3-Atto532-Biotin, and cytosine nucleotide or derivative thereof-N3-Atto532;
Combination 16: adenine nucleotide or derivative thereof-N3-CY5, guanine nucleotide or derivative thereof-N3-Biotin, thymine nucleotide or derivative thereof-N3-Atto532-Biotin, and cytosine nucleotide or derivative thereof-N3-Atto532;
Combination 17: adenine nucleotide or derivative thereof-N3-Atto647N, guanine nucleotide or derivative thereof-N3-Biotin, thymine cytosine nucleotide or derivative thereof-N3-Atto647N-Biotin, and cytosine nucleotide or derivative thereof-N3-Atto532;
Combination 18: adenine nucleotide or derivative thereof-N3-CY5, guanine nucleotide or derivative thereof-N3-Biotin, thymine nucleotide or derivative thereof-N3-Atto647N-Biotin, and cytosine nucleotide or derivative thereof-N3-Atto532;
Combination 19: adenine nucleotide or derivative thereof-N3-Atto647N, guanine nucleotide or derivative thereof-N3-Biotin, thymine nucleotide or derivative thereof-N3-CY5-Biotin, and cytosine nucleotide or derivative thereof-N3-Atto532;
Combination 20: adenine nucleotide or derivative thereof-N3-Atto532, guanine nucleotide or derivative thereof-N3-Biotin, thymine cytosine nucleotide or derivative thereof-N3-Atto532-Biotin, and cytosine nucleotide or derivative thereof-N3-Atto647N;
Combination 21: adenine nucleotide or derivative thereof-N3-Atto532, guanine nucleotide or derivative thereof-N3-Biotin, thymine cytosine nucleotide or derivative thereof-N3-Atto532-Biotin, and cytosine nucleotide or derivative thereof-N3-CY5;
Combination 22: adenine nucleotide or derivative thereof-N3-Atto647N, guanine nucleotide or derivative thereof-N3-Biotin, thymine cytosine nucleotide or derivative thereof-N3-Biotin, and cytosine nucleotide or derivative thereof-N3-Atto532;
Combination 23: adenine nucleotide or derivative thereof-N3-CY5, guanine nucleotide or derivative thereof-N3-Biotin, thymine nucleotide or derivative thereof-N3-Biotin, and cytosine nucleotide or derivative thereof-N3-Atto532;
Combination 24: adenine nucleotide or derivative thereof-N3-Atto532, guanine nucleotide or derivative thereof-N3-Biotin, thymine cytosine nucleotide or derivative thereof-N3-Biotin, and cytosine nucleotide or derivative thereof-N3-Atto647N;
Combination 25: adenine nucleotide or derivative thereof-N3-Atto532, guanine nucleotide or derivative thereof-N3-Biotin, thymine cytosine nucleotide or derivative thereof-N3-Biotin, and cytosine nucleotide or derivative thereof-N3-CY5,
optionally, in Combinations 1 to 14, the cleavable group —N3- in any one of the reversible terminators not having the specific binding label is replaced with —N3-SS—, and the cleavable group —N3-SS— in any one of the reversible terminators not having the specific binding label is replaced with —N3-; and/or
in Combinations 1 to 14, —N3-SS— is replaced with —SS—;
optionally, in Combinations 15 to 25, CY5 and/or Atto647N are/is replaced with GM656 or Alexa Fluor647; and/or Atto532 and/or Alexa Fluor532 are/is replaced with Atto Rho6G or CY3; or
in Combinations 15 to 25, CY5 and/or Atto647N are/is replaced with one of the blue light dyes.

10. The two-color sequencing method according to claim 9, wherein the working concentration of the adenine nucleotide or derivative thereof is from 10 nmol/L to 700 nmol/L, the working concentration of the thymine nucleotide or derivative thereof is from 5 nmol/L to 60 nmol/L, the working concentration of the cytosine nucleotide or derivative thereof is from 10 nmol/L to 700 nmol/L, and the working concentration of the guanine nucleotide or derivative thereof is from 0.05 nmol/L to 5 nmol/L.

11. The two-color sequencing method according to claim 1, wherein the nucleic acid molecules are single-copy nucleic acid templates.

12. The two-color sequencing method according to claim 1, wherein the method further comprises:
(f) repeating (a) to (e) one or more times to determine the sequence of the template nucleic acids.

13. A two-color sequencing kit, wherein the two-color sequencing kit at least comprises four types of reversible terminator and a first cleavage reagent,
the four types of reversible terminator each independently comprise: a nucleotide or derivative thereof, a labeling moiety, and a first cleavable group linking the nucleotide or derivative thereof and the labeling moiety, wherein the labeling moiety is selected from a fluorescent group and/or a specific binding label, at least two of the four types of modified nucleotide carry a first fluorescent group and a second fluorescent group, which are optically distinguishable, respectively, one or two of the four types of modified nucleotide carry the specific binding label, the specific binding label is capable of specifically binding to a corresponding ligand, and the first cleavage reagent is used to cleave the first cleavable group.

14. The two-color sequencing kit according to claim 13, wherein three of the four types of modified nucleotide carry the first fluorescent group and the second fluorescent group, which are optically distinguishable, respectively, the other of the four types of modified nucleotide carries the specific binding label, and one of the two types of modified nucleotide carrying the same fluorescent group comprises a second cleavable group, the second cleavable group being so located as to link the first cleavable group and the labeling moiety, and the second cleavable group having a higher cleavability than the first cleavable group, and
the kit further comprises a second cleavage reagent, the second cleavage reagent being used to cleave the second cleavable group.

15. The two-color sequencing kit according to claim 14, wherein the structures of the four types of reversible terminator satisfy one or more of the following combinations (1)-(4):
(1)_the structure of the first reversible terminator is: first nucleotide or derivative thereof-first cleavable group-first fluorescent group,
the structure of the second reversible terminator is: second nucleotide or derivative thereof-first cleavable group-specific binding label,
the structure of the third reversible terminator is: third nucleotide or derivative thereof-first cleavable group-specific binding label, and
the structure of the fourth reversible terminator is: fourth nucleotide or derivative thereof-first cleavable group-second fluorescent group;
(2)_the structure of the first reversible terminator is: first nucleotide or derivative thereof-first cleavable group-first fluorescent group,
the structure of the second reversible terminator is: second nucleotide or derivative thereof-first cleavable group-specific binding label,
the structure of the third reversible terminator is: third nucleotide or derivative thereof-first cleavable group-specific binding label, and
the structure of the fourth reversible terminator is: fourth nucleotide or derivative thereof-first cleavable group-second fluorescent group-specific binding label;
(3)_the structure of the first reversible terminator is: first nucleotide or derivative thereof-first cleavable group-first fluorescent group,
the structure of the second reversible terminator is: second nucleotide or derivative thereof-first cleavable group-specific binding label,
the structure of the third reversible terminator is: third nucleotide or derivative thereof-first cleavable group-second fluorescent group, and
the structure of the fourth reversible terminator is: fourth nucleotide or derivative thereof-first cleavable group-second cleavable group-first fluorescent group-specific binding label;
(4)_the structure of the first reversible terminator is: first nucleotide or derivative thereof-first cleavable group-first fluorescent group,
the structure of the second reversible terminator is: second nucleotide or derivative thereof-first cleavable group-second fluorescent group-specific binding label,
the structure of the third reversible terminator is: third nucleotide or derivative thereof-first cleavable group-second fluorescent group, and
the structure of the fourth reversible terminator is: fourth nucleotide or derivative thereof-first cleavable group-second cleavable group-first fluorescent group;
wherein in each combination, the first nucleotide or derivative thereof, the second nucleotide or derivative thereof, the third nucleotide or derivative thereof, and the fourth nucleotide or derivative thereof are selected from one of adenine or derivative thereof, thymine or derivative thereof, cytosine or derivative thereof, or guanine or derivative thereof, and the first nucleotide or derivative thereof, the second nucleotide or derivative thereof, the third nucleotide or derivative thereof, and the fourth nucleotide or derivative thereof are different from each other.

16. The two-color sequencing kit according to claim 15, wherein the first cleavable group is selected from groups comprising at least one of —N3-, —CH₂F, —C(O)NHBu, —CHF₂, —C(O)OH, —C6-, —ArC6-, and/or a disulfide bond.

17. The two-color sequencing kit according to claim 15, wherein the first fluorescent group and the second fluorescent group are each independently selected from one of a red fluorescent group, a green fluorescent group, and a blue fluorescent group.

18. The two-color sequencing kit according to claim 13, wherein the specific binding label is selected from at least one of biotin, digoxin, hapten, or functional derivatives thereof.

19. The two-color sequencing kit according to claim 13, wherein the reversible terminators comprise at least one group shown in the following Combinations 1 to 25:
Combination 1: adenine nucleotide or derivative thereof-N3-Atto647N, guanine nucleotide or derivative thereof-N3-SS-Atto647N, thymine nucleotide or derivative thereof-N3-Biotin, and cytosine nucleotide or derivative thereof-N3-Atto532;
Combination 2: adenine nucleotide or derivative thereof-N3-CY5, guanine nucleotide or derivative thereof-N3-SS-Atto647N, thymine nucleotide or derivative thereof-N3-Biotin, and cytosine nucleotide or derivative thereof-N3-Atto532;
Combination 3: adenine nucleotide or derivative thereof-N3-CY5, guanine nucleotide or derivative thereof-N3-SS-CY5, thymine nucleotide or derivative thereof-N3-Biotin, and cytosine nucleotide or derivative thereof-N3-Atto532;
Combination 4: adenine nucleotide or derivative thereof-N3-Atto647N, guanine nucleotide or derivative thereof-N3-SS-CY5, thymine nucleotide or derivative thereof-N3-Biotin, and cytosine nucleotide or derivative thereof-N3-Atto532;
Combination 5: adenine nucleotide or derivative thereof-N3-Atto647N, guanine nucleotide or derivative thereof-N3-SS-Atto647N, thymine nucleotide or derivative thereof-N3-Atto532-Biotin, and cytosine nucleotide or derivative thereof-N3-Atto532;
Combination 6: adenine nucleotide or derivative thereof-N3-Atto647N, guanine nucleotide or derivative thereof-N3-SS-CY5, cytosine nucleotide or derivative thereof-N3-Atto532-Biotin, and cytosine nucleotide or derivative thereof-N3-Atto532;
Combination 7: adenine nucleotide or derivative thereof-N3-CY5, guanine nucleotide or derivative thereof-N3-SS-Atto647N, thymine nucleotide or derivative thereof-N3-Atto532-Biotin, and cytosine nucleotide or derivative thereof-N3-Atto532;
Combination 8: adenine nucleotide or derivative thereof-N3-CY5, guanine nucleotide or derivative thereof-N3-SS-CY5, thymine nucleotide or derivative thereof-N3-Atto532-Biotin, and cytosine nucleotide or derivative thereof-N3-Atto532;
Combination 9: adenine nucleotide or derivative thereof-N3-Atto647N, guanine nucleotide or derivative thereof-N3-SS-Atto532, thymine nucleotide or derivative thereof-N3-Biotin, and cytosine nucleotide or derivative thereof-N3-Atto532;
Combination 10: adenine nucleotide or derivative thereof-N3-CY5, guanine nucleotide or derivative thereof-N3-SS-Atto532, thymine nucleotide or derivative thereof-N3-Biotin, and cytosine nucleotide or derivative thereof-N3-Atto532;
Combination 11: adenine nucleotide or derivative thereof-N3-Atto532, guanine nucleotide or derivative thereof-N3-SS-Atto532, cytosine nucleotide or derivative thereof-N3-Biotin, and cytosine nucleotide or derivative thereof-N3-Atto647N;
Combination 12: adenine nucleotide or derivative thereof-N3-Atto532, guanine nucleotide or derivative thereof-N3-SS-Atto532, thymine nucleotide or derivative thereof-N3-Biotin, and cytosine nucleotide or derivative thereof-N3-CY5;
Combination 13: adenine nucleotide or derivative thereof-N3-Atto532, guanine nucleotide or derivative thereof-N3-SS-Atto532, cytosine nucleotide or derivative thereof-N3-Atto647N-Biotin, and cytosine nucleotide or derivative thereof-N3-Atto647N;
Combination 14: adenine nucleotide or derivative thereof-N3-Atto532, guanine nucleotide or derivative thereof-N3-SS-Atto532, thymine cytosine nucleotide or derivative thereof-N3-CY5-Biotin, and cytosine nucleotide or derivative thereof-N3-CY5;
Combination 15: adenine nucleotide or derivative thereof-N3-Atto647N, guanine nucleotide or derivative thereof-N3-Biotin, thymine nucleotide or derivative thereof-N3-Atto532-Biotin, and cytosine nucleotide or derivative thereof-N3-Atto532;
Combination 16: adenine nucleotide or derivative thereof-N3-CY5, guanine nucleotide or derivative thereof-N3-Biotin, thymine nucleotide or derivative thereof-N3-Atto532-Biotin, and cytosine nucleotide or derivative thereof-N3-Atto532;
Combination 17: adenine nucleotide or derivative thereof-N3-Atto647N, guanine nucleotide or derivative thereof-N3-Biotin, thymine cytosine nucleotide or derivative thereof-N3-Atto647N-Biotin, and cytosine nucleotide or derivative thereof-N3-Atto532;
Combination 18: adenine nucleotide or derivative thereof-N3-CY5, guanine nucleotide or derivative thereof-N3-Biotin, thymine nucleotide or derivative thereof-N3-Atto647N-Biotin, and cytosine nucleotide or derivative thereof-N3-Atto532;
Combination 19: adenine nucleotide or derivative thereof-N3-Atto647N, guanine nucleotide or derivative thereof-N3-Biotin, thymine nucleotide or derivative thereof-N3-CY5-Biotin, and cytosine nucleotide or derivative thereof-N3-Atto532;
Combination 20: adenine nucleotide or derivative thereof-N3-Atto532, guanine nucleotide or derivative thereof-N3-Biotin, thymine cytosine nucleotide or derivative thereof-N3-Atto532-Biotin, and cytosine nucleotide or derivative thereof-N3-Atto647N;
Combination 21: adenine nucleotide or derivative thereof-N3-Atto532, guanine nucleotide or derivative thereof-N3-Biotin, thymine cytosine nucleotide or derivative thereof-N3-Atto532-Biotin, and cytosine nucleotide or derivative thereof-N3-CY5;
Combination 22: adenine nucleotide or derivative thereof-N3-Atto647N, guanine nucleotide or derivative thereof-N3-Biotin, thymine cytosine nucleotide or derivative thereof-N3-Biotin, and cytosine nucleotide or derivative thereof-N3-Atto532;
Combination 23: adenine nucleotide or derivative thereof-N3-CY5, guanine nucleotide or derivative thereof-N3-Biotin, thymine nucleotide or derivative thereof-N3-Biotin, and cytosine nucleotide or derivative thereof-N3-Atto532;
Combination 24: adenine nucleotide or derivative thereof-N3-Atto532, guanine nucleotide or derivative thereof-N3-Biotin, thymine cytosine nucleotide or derivative thereof-N3-Biotin, and cytosine nucleotide or derivative thereof-N3-Atto647N;
Combination 25: adenine nucleotide or derivative thereof-N3-Atto532, guanine nucleotide or derivative thereof-N3-Biotin, thymine cytosine nucleotide or derivative thereof-N3-Biotin, and cytosine nucleotide or derivative thereof-N3-CY5,
optionally, in Combinations 1 to 14, the cleavable group —N3- in any one of the reversible terminators not having the specific binding label is replaced with —N3-SS—, and the cleavable group —N3-SS— in any one of the reversible terminators not having the specific binding label is replaced with —N3-; and/or
in Combinations 1 to 14, —N3-SS— is replaced with —SS—;
optionally, in Combinations 15 to 25, CY5 and/or Atto647N are/is replaced with GM656 or Alexa Fluor647; and/or Atto532 and/or Alexa Fluor532 are/is replaced with Atto Rho6G or CY3; or
in Combinations 15 to 25, CY5 and/or Atto647N are/is replaced with one of the blue light dyes.

20. The two-color sequencing kit according to claim 19, wherein the working concentration of the adenine nucleotide or derivative thereof is from 10 nmol/L to 700 nmol/L, the working concentration of the thymine nucleotide or derivative thereof is from 5 nmol/L to 60 nmol/L, the working concentration of the cytosine nucleotide or derivative thereof is from 10 nmol/L to 700 nmol/L, and the working concentration of the guanine nucleotide or derivative thereof is from 0.05 nmol/L to 5 nmol/L.
